(19) United States
(12) Patent Application Publication
(10) Pub. No.: US 2019/0352634 A1
(43) Pub. Date: Nov. 21, 2019

(54) CRISPR RNA

(71) Applicant: Oxford University Innovation Limited, Oxford (GB)

(72) Inventors: Tudor Alexandru FULGA, Oxford (Oxfordshire) (GB); Quentin FERRY, Oxford (Oxfordshire) (GB)

(21) Appl. No.: 16/476,989

(22) PCT Filed: Jan. 11, 2018
(86) PCT No.: PCT/GB2018/050065

§ 371 Date: Jul. 10, 2019

(30) Foreign Application Priority Data
Jan. 11, 2017 (GB) 1700460.7
Jan. 24, 2017 (GB) 1701180.0

(51) Int. Cl. C12N 15/10; C12N 15/113; C12N 15/63

(52) U.S. Cl. CPC C12N 15/102; C12N 15/113; C12N 2310/3519; C12N 2310/20; C12N 15/63

(57) ABSTRACT

The present invention relates to an inducible CRISPR RNA comprising a spacer-blocking element, a cleavable loop element and a CRISPR sgRNA comprising a spacer element (guide sequence). The invention also provides methods of using inducible CRISPR RNA/CRISPR enzyme complexes.

The present invention relates to an inducible CRISPR RNA comprising a spacer-blocking element, a cleavable loop element and a CRISPR sgRNA comprising a spacer element (guide sequence). The invention also provides methods of using inducible CRISPR RNA/CRISPR enzyme complexes.

Synthetic biology in mammalian systems holds great promise for both deciphering the wiring of natural gene networks (GN) as well as engineering cells for therapeutic benefit [1, 2]. This process relies on the characterisation and assembly of biological parts into de novo synthetic pathways designed to redirect or enhance the scope of naturally evolved cellular behaviours [3].

Adding to a growing list of available standardised components, the type-II clustered regularly interspaced short palindromic repeats (CRISPR):Cas9 from Streptococcus pyogenes (Sp) has been recently repurposed to create programmable transcription regulators (CRISPR-TR) in mammalian cells [4, 5]. This conceptual framework takes advantage of the ability to direct a nuclease-deficient Cas9 (dCas9) to any given N₂₀NGG (PAM) DNA sequence in the genome by simply reprogramming its associated single guide RNA (sgRNA). Consequently, the output expression of any gene of interest can be controlled by tethering various effector domains to the sgRNA-dCas9 complex and targeting them near transcription start sites (TSS) [4, 6, 7].

A critical dimension in synthetic biology is the design of inducible parts, enabling construction of complex gene circuits responsive to exogenous cues and/or endogenous metabolites. Although elegant chemically-inducible and photoactivated CRISPR:Cas9 solutions have recently been reported in mammalian cells, these systems have been restricted to post-translational control of dCas9 function or dCas9-effector tethering [8]. Because dCas9 binds without discrimination to all sgRNAs regardless of their cognate target, such approaches cannot be easily scaled up to create circuits involving orthogonal transcriptional programs acting on multiple genes. While Cas9 variants with divergent PAM specificities can provide an orthogonal framework for transcription activators (CRISPR-TA [9]), their utility in the design of inducible systems is mitigated by the necessity of extensive protein engineering and the metabolic costs associated with protein delivery.

To address one or more of the above-mentioned limitations, a versatile inducible CRISPR RNA system has been developed based on engineering of the CRISPR sgRNA.

The present invention provides an inducible CRISPR RNA comprising a spacer-blocking element, a cleavable loop element and a CRISPR sgRNA comprising a spacer element (guide sequence). The spacer-blocking element is at least partially complementary to the spacer element, and the loop element and spacer-blocking element are capable of forming a stem-loop structure which is capable of blocking the spacer element. Upon programmed cleavage of the loop element conditioned on the presence of an inducer, the spacer blocking element is liberated, thus allowing the spacer element (guide sequence) to direct an associated CRISPR enzyme to a target DNA.

Complexes formed between the inducible CRISPR RNA/CRISPR enzyme may comprise one or more functional domains which, when juxtaposed to a target DNA, promote a desired functional activity, e.g. transcriptional activation of a given target gene.

Such inducible CRISPR RNAs may be used to couple multiple target genes and inducers in orthogonal pairs, and to subsequently assemble gene regulatory modules demonstrating synchronous and asynchronous transcriptional programs. This ‘plug and play’ approach will be a valuable addition to the synthetic biology toolkit, facilitating the understanding of natural gene circuits as well as the design of cell-based therapeutic strategies.

The invention also provides methods of using the inducible CRISPR RNA and inducible CRISPR RNA/CRISPR enzyme complexes for genome editing, epigenetic alteration, base editing, DNA labelling and lineage tracing throughout development or in disease states.

In one embodiment, the invention provides an inducible CRISPR RNA comprising:
(i) a spacer-blocking element;
(ii) a cleavable loop element; and
(iii) a CRISPR sgRNA comprising a spacer element;
   wherein (i)-(iii) are arranged 5′-3′ in the above order in the inducible CRISPR RNA, wherein the spacer-blocking element has a nucleotide sequence which is at least partially complementary to that of the spacer element, and wherein the spacer-blocking element, cleavable loop element and spacer element are capable of forming a stem-loop structure.

In a further embodiment, the invention provides a method for the induced targeting of a CRISPR complex to a target DNA in a host cell, the method comprising the steps:
(i) expressing in the host cell:
   (a) an inducible CRISPR RNA of the invention, wherein the nucleotide sequence of the spacer element is fully or partially complementary to a region of the target DNA; and
   (b) a CRISPR enzyme, such that the inducible CRISPR RNA and CRISPR enzyme form a CRISPR complex; and
(ii) inducing, at a desired time, cleavage of the cleavable loop element of the inducible CRISPR RNA,
   thus allowing the spacer element to target the CRISPR complex to the target DNA.

In a further embodiment, the invention provides a method for inducibly targeting a functional domain to a target DNA in a host cell, the method comprising the steps:
(i) expressing in the host cell:
   (a) an inducible CRISPR RNA of the invention, wherein the nucleotide sequence of the spacer element is fully or partially complementary to a region of the target DNA; and
   (b) a CRISPR enzyme,
such that the inducible CRISPR RNA and CRISPR enzyme form a CRISPR complex, wherein the CRISPR complex comprises one or more functional domains; and
(ii) inducing, at a desired time, cleavage of the cleavable loop element of the CRISPR RNA,
thus allowing the spacer element to direct binding of the CRISPR complex to the target DNA, and thus targeting the one or more functional domains to the target DNA.

In yet a further embodiment, the invention provides a method for inducing transcription of a target gene in a target DNA in a host cell, the method comprising the steps:
(i) expressing in the host cell:
   (a) an inducible CRISPR RNA of the invention, wherein the nucleotide sequence of the spacer element is fully or partially complementary to a region of the target DNA in the vicinity of the target gene; and
   (b) a CRISPR enzyme,
such that the inducible CRISPR RNA and CRISPR enzyme form a CRISPR complex, wherein the CRISPR complex comprises one or more activator domains; and
(ii) inducing, at a desired time, cleavage of the cleavable loop element of the inducible CRISPR RNA,
   thus allowing the spacer element to direct binding of the CRISPR complex to the target DNA and thus targeting the one or more activator domains to the region of the target DNA in the vicinity of the target gene, thus inducing transcription of the target gene.

In yet a further embodiment, the invention provides a method for repressing transcription of a target gene in a target DNA in a host cell, the method comprising the steps:
(i) expressing in the host cell:
   (a) an inducible CRISPR RNA of the invention, wherein the nucleotide sequence of the spacer element is fully or partially complementary to a region of the target DNA in the vicinity of the target gene; and
   (b) a CRISPR enzyme,
such that the inducible CRISPR RNA and CRISPR enzyme form a CRISPR complex, wherein the CRISPR complex comprises one or more repressor domains; and
(ii) inducing, at a desired time, cleavage of the cleavable loop element of the inducible CRISPR sgRNA,
   thus allowing the spacer element to direct binding of the CRISPR complex to the target DNA and thus targeting the one or more repressor domains to the region of the target DNA in the vicinity of the target gene, thus repressing transcription of the target gene.

In one embodiment, the invention provides an inducible CRISPR RNA. CRISPR is an acronym for Clustered, Regularly Interspaced, Short, Palindromic Repeats. The CRISPR RNA of the invention may also be called a modified sgRNA or extended sgRNA.

The RNA is made up of ribonucleotides A, G, T and U. Modified ribonucleotides may also be used.

The inducible CRISPR RNA comprises a spacer-blocking element. The spacer-blocking element has a ribonucleotide sequence which is fully or partially complementary to that of the spacer element in the CRISPR sgRNA.

In some embodiments, the degree of ribonucleotide sequence identity between the spacer-blocking element and the spacer element is at least 50%, 60%, 70%, 75%, 80%, 85%, 90%, 95%, 98% or 99%, or is 100%.

The length of the spacer-blocking element is preferably 10-30 or 15-30, more preferably 10-25 or 15-25, and most preferably 18-22 or 20 nucleotides. Preferably, the spacer-blocking element is the same length as the spacer element ±0, 1 or 2 nucleotides.

In some embodiments, the spacer-blocking element is 1-20, 1-10 or 1-5 nucleotides longer than the spacer element at the 5′-end of the spacer-blocking element, thus producing a non-base-paired 5′-overhang. In some embodiments, the spacer-blocking element is 1-20, 1-10 or 1-5 nucleotides shorter than the spacer element at the 5′-end of the spacer-blocking element, thus producing a non-base-paired exposed region of the spacer element.

The structural free energy (i.e. the separation free energy) of the binding of the spacer-blocking element to the spacer element may be predicted using the NUPACK suite (J. N. Zadeh et al. NUPACK: analysis and design of nucleic acid systems. J. Comput. Chem., 32:170-173, 2011).

In some embodiments, the spacer-blocking element does not consist of or comprise a helix from a cis-acting hammerhead ribozyme. In some embodiments, the spacer-blocking element is not helix I from a cis-acting hammerhead ribozyme.

Percentage amino acid sequence identities and nucleotide sequence identities may be obtained using the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST). Preferably the standard or default alignment parameters are used.

Standard protein-protein BLAST (blastp) may be used for finding similar sequences in protein databases. Like other BLAST programs, blastp is designed to find local regions of similarity. When sequence similarity spans the whole sequence, blastp will also report a global alignment, which is the preferred result for protein identification purposes. Preferably the standard or default alignment parameters are used. In some instances, the “low complexity filter” may be taken off.

BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules (see Altschul et al. (1997) supra). When utilizing BLAST, Gapped BLAST, PSI-BLAST, the default parameters of the respective programs may be used.

With regard to nucleotide sequence comparisons, MEGABLAST, discontiguous-megablast, and blastn may be used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention.

The BLAST nucleotide algorithm (https://blast.ncbi.nlm.nih.gov/Blast.cgi?PAGE_TYPE=BlastSearch&BLAST_SPEC=bla st2seq&LINK_LOC=align2seq) finds similar sequences by breaking the query into short subsequences called words. The program identifies the exact matches to the query words first (word hits). The BLAST program then extends these word hits in multiple steps to generate the final gapped alignments. In some embodiments, the BLAST nucleotide searches can be performed with the BLASTN program, preferably with the parameters: Expect threshold 10; wordsize 28; match/mismatch scores 1,-2; Gap costs linear.

One of the important parameters governing the sensitivity of BLAST searches is the word size. The most important reason that blastn is more sensitive than MEGABLAST is that it uses a shorter default word size (11). Because of this, blastn is better than MEGABLAST at finding alignments to related nucleotide sequences from other organisms. The word size is adjustable in blastn and can be reduced from the default value to a minimum of 7 to increase search sensitivity.

A more sensitive search can be achieved by using the newly-introduced discontiguous megablast page (www.ncbi.nlm.nih.gov/Web/Newsltr/FallWinter02/blastlab.html). This page uses an algorithm which is similar to that reported by Ma et al. (Bioinformatics. 2002 March; 18(3): 440-5). Rather than requiring exact word matches as seeds for alignment extension, discontiguous megablast uses non-contiguous words within a longer window of template. In coding mode, the third base wobbling is taken into consideration by focusing on finding matches at the first and second codon positions while ignoring the mismatches in the third position. Searching in discontiguous MEGABLAST using the same word size is more sensitive and efficient than standard blastn using the same word size. Parameters unique for discontiguous megablast are: word size: 11 or 12; template: 16, 18, or 21; template type: coding (0), non-coding (1), or both (2).

The stem-loop structure which is formed or is capable of being formed between the spacer-blocking element and the spacer element may comprise one or more bulges.

Such bulges promote separation of the spacer-blocking element and the spacer element after cleavage of the loop element (thus facilitating hybridisation of the spacer element to the target gene).

As used herein, the term “bulges” refers to regions of the stem-loop structure which are formed between the spacer-blocking element and the spacer element which do not have 100% nucleotide sequence identity.

They may be viewed as discontinuities in the Watson-Crick hybridisation between the spacer-blocking element and the spacer element.

The number, length and positions of the bulges may all vary. The choices are dictated by the overall stability of the stem-loop structure. If the RNA secondary structure prediction algorithm (e.g. the NUPCAK discussed above) predicts that the number of matched nucleotides is not enough to guarantee stable formation of the hairpin, then this number (or the size of the bulge) may be adjusted until the desired stability is obtained.

Preferably, the stem-loop structure comprises 1, 2, 3, 4 or 5 bulges, more preferably 1, 2 or 3 bulges, and most preferably 2 bulges.

The bulges are preferably independently 1, 2, 3, 4, 5, 6, 7 or 8 nucleotides in length, most preferably 2 or 3 nucleotides in length.

If there is more than one bulge, there are preferably independently 2, 3 or 4 (complementary) nucleotides between each bulge.

In some embodiments, less stable stem loops might be created by using intercalated binding patterns, e.g. a 1 nucleotide mismatch every two matched nucleotides. Such patterns may continue for 1-20, 1-10, 1-6 or 1-3 nucleotides in the spacer element, for example.

Preferably, there are 1, 2 or 3 (most preferably 1) (complementary) nucleotides between the 5′-most bulge and the loop element.

In a particularly preferred embodiment, the spacer element is 19 nucleotides in length; the spacer-blocking element is 20 nucleotides in length; wherein when the spacer-blocking element is hybridised to the spacer element, two bulges of 2 non-complementary base pairs are formed. Preferably, the two bulges are separated by a region of 4 complementary nucleotides.

The inducible CRISPR RNA also comprises a cleavable loop element. The cleavable loop element acts as a linker to join the spacer-blocking element and the spacer element.

The spacer-blocking element, cleavable loop element and the spacer element form a hairpin loop or stem loop structure.

The nucleotide sequence of the loop will be such that it allows the above-mentioned hairpin loop or stem loop structure to form. In general, the nucleotide sequence of the loop structure will have no sequence identity with the spacer-blocking element or with the spacer element.

The cleavable loop element is preferably 1-30, 1-20 or 1-10 nucleotides in length, most preferably 10-20 or 10-30 nucleotides in length.

In some embodiments, the cleavable loop element comprises or consists of a single-stranded contiguous stretch of ribonucleotides. In other embodiments, the cleavable loop element comprises or consists of a stem-loop structure.

In yet other embodiments, the cleavable loop element comprises or consists of a loop-stem-loop structure (e.g. wherein the Csy4 or Cas6A RNA motif is incorporated into the loop structure) or a loop-stem-loop-stem-loop structure.

In some embodiments, the 5′-end of the spacer element and the corresponding (hybridising) section of the spacer-blocking element may form part of the loop.

The loop element between the spacer-blocking element and the spacer element is a cleavable loop element. As used herein, the term “cleavable loop element” means that it is possible to cleave, i.e. cut, the ribonucleotide strand which forms the loop element into two or more parts.

The loop element is, in general, one which is capable of being bound by a ribonucleotide binding moiety in a sequence-specific manner.

In some embodiments, this ribonucleotide sequence-specific binding moiety is a polypeptide. In other embodiments, this ribonucleotide sequence-specific binding moiety is an oligonucleotide.

Preferably, the loop element comprises a cleavage site for an endoribonuclease, more preferably an endoribonuclease which cleaves RNA at or within a defined ribonucleotide sequence motif, i.e. the loop element comprises a cleavage site for a motif-specific endoribonuclease. (This may be contrasted with endonucleases such as RNAseH which are capable of cleaving DNA:RNA hybrids of any sequence.)

More preferably, the cleavable loop element comprises a cleavage site for an ssRNA endoribonuclease. (This may be contrasted with endonucleases such as RNAseH which cleave a double-stranded template.) Preferably, the cleavable loop element comprises a cleavage site for a motif-specific ssRNA endoribonuclease.

Examples of well-characterized CRISPR endoribonucleases which may be used include the following (from Hochstrasser and Doudna, TIBS vol. 40, Issue 1, p 58-66, January 2015):

| Subtype | Name | Organism(s) | Other name(s) |
|---|---|---|---|
| I-A | PhoCas6nc | Pyrococcus horikoshii | Cas6a |
| | SsoCas6-1A | Sulfolobus solfataricus | Sso2004 |
| | | | (Cas6-1 family), |
| | | | SsCas6 |
| | SsoCas6-1B | | Sso1437 |
| | | | (Cas6-1 family), |
| | | | SsoCas6 |
| | SsoCas6-3 | | Sso1422 (Cas6-3) |
| I-B | MmaCas6b | Methanococcus | Mm, Cas6b |
| | | maripaludis | |
| I-C | Cas5c | Bacillus halodurans, | Cas5d |
| | | Mannheimia | |
| | | succiniciproducens, | |
| | | Streptococcus pyogenes, | |
| | | Xanthomonas oryzae | |
| I-E | TthCas6e | Thermus thermophilus | TTHB192, Cse3 |
| | EcoCas6e | Escherichia coli | CasE |
| | EcoCas5e | | CasD |
| I-F | PaeCas6f | Pseudomonas aeruginosa | Csy4 |
| III-A | SepCas6 | Staphylococcus epidermidis | |
| III-B | PfuCas6-1 | Pyrococcus furiosus | PfCas6 |
| | PfuCas6-3nc | | PfCas6-3 |
| I-B? | TthCas6B | T. thermophilus | TTHB231 |
| Orphan | TthCas6A | | TTHB78 |

In some embodiments, the endoribonuclease is a member of the Cas6 superfamily, preferably Cas6A (e.g. Hong Li (2015), Structure, January 6; 23(1):13-20).

In other embodiments, the endoribonuclease is Csy4 [15, 16]. Csy4 is a CRISPR-associated endoribonuclease from Pseudomonas aeruginosa which recognises and cleaves the 16 nt core of a 28 nucleotide RNA stem-loop. Csy4 is also known as Cas6f.

In other embodiments, the endoribonuclease is Cpf1. This has been shown to process pre-creRNA transcripts (Zetsche, B. et al. (2016), “Multiplex gene editing by CRISPR-Cpf1 using a single crRNA array”, Nature Biotechnology (2016) doi:10.1038/nbt.3737).

In yet other embodiments, the cleavable loop element is one which is cleavable by a hammerhead ribozyme, preferably by an allosteric self-cleaving hammerhead ribozyme (aHHRz).

Hammerhead ribozymes are RNA molecule motifs that catalyse reversible cleavage and joining reactions at a specific site within an RNA molecule. The self-cleavage reactions are part of a rolling circle replication mechanism. The hammerhead sequence is sufficient for self-cleavage and acts by forming a conserved three-dimensional tertiary structure.

Previous studies have shown that aHHRz can be effectively used for the construction of ligand-controlled synthetic circuits [27-29].

In some embodiments, the cleavable loop element comprises a HHRz coupled to an aptamer domain, preferably a ligand-sensing aptamer domain. Ligand binding may control HHRz folding and three-dimensional tertiary structure, hence controlling release of the connected spacer-blocking element. The ligand may, for example, be a protein, nucleotide or small molecule ligand.

The inducible CRISPR RNA also comprises a CRISPR sgRNA comprising a spacer element. The term “sgRNA” refers to a single-guide RNA. It is a chimeric RNA which replaces the crRNA/tracrRNA which are used in the native CRISPR/Cas systems (e.g. Jinek, M. et al. (2012), “A programmable dual-RNA-guided DNA endonuclease in adaptive bacterial immunity”, Science 337, 816-821). The term sgRNA is well accepted in the art.

The sgRNA comprises a spacer element. The spacer element is also known as a spacer segment or guide sequence. The terms spacer element, spacer segment and guide sequence are used interchangeably.

The sgRNA comprises a region which is capable of forming a complex with a CRISPR enzyme, e.g. a CRISPR endonuclease, e.g. Cas9. The sgRNA comprises, from 5′ to 3′, a spacer element which is programmable (i.e. the sequence may be changed to target a complementary DNA target), followed by the sgRNA scaffold.

The sgRNA scaffold may technically be divided further into modules whose names and coordinates are well known in the art (e.g. Briner, A. E. et al. (2014). “Guide RNA functional modules direct cas9 activity and orthogonality”. Molecular Cell, 56(2), 333-339).

The spacer element is a stretch of contiguous ribonucleotides whose sequence is fully or partially complementary to the target DNA (i.e. the protospacer).

The target nucleic acid may be DNA or RNA. Preferably, the target nucleic acid is DNA.

The target DNA is preferably eukaryotic DNA. The target DNA may be any DNA within the host cells. The target DNA may, for example, be chromosomal DNA, mitochondrial DNA, plastid DNA, plasmid DNA or vector DNA, as desired.

In some embodiments, the target may be a regulatory element, e.g. an enhancer, promoter, or terminator sequence. In other embodiments, the target DNA is an intron or exon in a polypeptide-coding sequence.

In some preferred embodiments, the target DNA is selected such that, upon binding of the sgRNA, the one or more functional domains which are present in the CRISPR complex (either attached via the sgRNA or to the CRISPR enzyme) are in a spatial orientation which allows the functional domain(s) to function in its attributed function.

The length of the spacer element is preferably 8-30, more preferably 8-25 and most preferably 9-23 nucleotides.

The degree of sequence identity between spacer element and the target DNA is preferably at least 50%, 60%, 70%, 75%, 80%, 85%, 90%, 95%, 98% or 99%, or is 100%.

The gene DNA will be associated with a PAM site (e.g. NGG) which must be flanking the targeted DNA for the CRISPR complex to be able to act on the target.

The spacer-blocking element, loop element and spacer element are capable of forming a stem-loop structure. As used herein, the term “stem-loop structure” is also known as a hairpin-loop structure.

The degree of sequence identity between the spacer-blocking element and the spacer element results in the hybridisation of the spacer-blocking element to the spacer element due to Watson-Crick base pairing.

This “blocking” of the spacer element leads to the result that the spacer element, when present in this configuration, cannot bind to its target (protospacer) DNA.

Antisense oligonucleotides (ASOs) have emerged as a highly versatile class of compounds that can be safely and efficiently delivered in both cells and organisms to alter gene expression (up- and down-regulation) and to interfere with post-transcriptional RNA processing (e.g. splicing, microRNA regulation, etc.) [18, 19].

Spacer-blocking element release mechanisms which are responsive to short ASOs may be engineered to provide a means for temporal exogenous control of the sgRNAs of the invention. This strategy relies on the ability of ssDNA ASOs to bind to complementary loop elements and to engage nuclear RNase-H mediated cleavage of the RNA strand in the resulting DNA:RNA hybrid, thus releasing the spacer-blocking element (and facilitating gene expression).

In some embodiments, therefore, the loop element is an element to which an antisense oligonucleotide may be bound. Preferably, the antisense oligonucleotide is a single-stranded DNA (ssDNA) oligonucleotide.

In some embodiments, the length of the cleavable loop element is a length to which nuclear RNase-H may bind and cleave a DNA:RNA hybrid formed between the loop and an antisense oligonucleotide.

With regard to loop elements to which antisense oligonucleotides may be bound, the loop element is preferably 6-40 nucleotides in length, more preferably 10-30, even more preferably 12-25 nucleotides in length, and most preferably about 14 nucleotides in length.

With regard to the length of the antisense oligonucleotide, this is preferably 10-40 nucleotides in length, more preferably 12-30, even more preferably 15-25 nucleotides in length.

In some embodiments, the antisense oligonucleotide is 12-16 nucleotides, more preferably about 14 nucleotides in length.

The degree of sequence complementarity between antisense oligonucleotide and the loop element is preferably at least 80%, 85%, 90%, 95%, 98% or more preferably it is 100%.

microRNAs (miRNAs) are short ˜22 nucleotide single-stranded non-coding RNAs which play essential roles in post-transcriptional control of gene expression. After biogenesis, mature miRNAs are loaded into the Argonaute (Ago) protein, which together with a number of co-factors form the miRISC complex. Guided by the miRNAs, the miRISC complex can target mRNAs bearing a sequence fully- or partially-complementary to the miRNA, termed a miRNA responsive element (MRE); this initiates transcript degradation. Previous studies have shown that the miRISC complex will cleave the mRNA target when the MRE is fully-complementary to the miRNA sequence, a function mediated exclusively by Ago2 in mammalian cells.

Considering the length of miRNA molecules and the cleaving properties of miRNA-loaded Ago, this miRNA/MRE system is usable in the context of the current invention as a spacer element release mechanism to generate miRNA-responsive sgRNAs.

In some embodiments, therefore, the cleavable loop element comprises a miRNA responsive element (MRE).

The length of the miRNA is preferably 20-24, more preferably 21-23, and most preferably 22 nucleotides in length.

The length of the MRE is a length to which a miRNA is capable of binding. The length of the MRE is preferably 20-24, more preferably 21-23, and most preferably 22 nucleotides in length.

The degree of sequence complementarity between the miRNA and the MRE is preferably at least 80%, 85%, 90%, 95%, 98% or 99%; more preferably it is 100%.

The CRISPR enzyme is one which is capable of forming a complex with the inducible CRISPR RNA (preferably with the CRISPR sgRNA). The CRISPR enzyme is one which, when complexed with an inducible CRISPR RNA of the invention or a CRISPR sgRNA, is capable of targeting the thus-produced complex to a target DNA which has a nucleotide sequence which is complementary to that of the spacer element in the sgRNA.

In some embodiments, the CRISPR enzyme is nuclease-deficient.

In other embodiments, the CRISPR enzyme has nuclease, preferably endonuclease, activity.

In some embodiments, the CRISPR enzyme is a Type II CRISPR system enzyme. In some embodiments, the CRISPR enzyme is Cas9 or a Cas9-like polypeptide. In some embodiments, the Cas9 enzyme is derived from S. pneumoniae, S. pyogenes, or S. thermophilus Cas9, or a variant thereof.

In some embodiments, the CRISPR enzyme is codon-optimized for expression in a eukaryotic cell.

In some embodiments, the aim of the complex is to target functional domain(s) to the desired target DNA; the aim is not to cleave the target DNA. Consequently, there is no need for the CRISPR enzyme to possess any endonuclease activity. In such embodiments, it is in fact desirable that the CRISPR enzyme does not have any or any significant endonuclease activity.

Preferably, the CRISPR enzyme is a catalytically-inactive or nuclease-deficient enzyme.

Preferably, the CRISPR enzyme is an enzyme which has no or substantially no endonuclease activity. Lack of nuclease activity may be assessed using a Surveyor assay to detect DNA repair events (Pinera et al. Nature Methods (2013) 10(10):973-976). The CRISPR enzyme is unable to cleave dsDNA but it retains the ability to target and bind the DNA.

In some embodiments, the CRISPR enzyme has no detectable nuclease activity. The CRISPR enzyme may, for example, be one with a diminished nuclease activity or one whose nuclease activity has been inactivated.

The CRISPR enzyme may, for example, have about 0% of the nuclease activity of the non-mutated or wild-type Cas9 enzyme; less than 3% or less than 5% of the nuclease activity of the non-mutated or wild-type Cas9 enzyme. The non-mutated or wild-type Cas9 enzyme may, for example, be SpCas9.

Reducing the level of nuclease activity is possible by introducing mutations into the RuvC and HNH nuclease domains of the SpCas9 and orthologs thereof. For example utilising one or more mutations in a residue selected from the group consisting of D10, E762, H840, N854, N863, or D986; and more preferably introducing one or more of the mutations selected from the group consisting DI0A, E762A, H840A, N854A, N863A or D986A. A preferred pair of mutations is DI0A with H840A; more preferred is DI0A with N863A of SpCas9 and orthologs thereof.

In some embodiments, the CRISPR enzyme is dCas9 enzyme. In some embodiments, the CRISPR enzyme is a nuclease-deficient Cpf1 (dCpf1).

In other embodiments, the CRISPR enzyme is not nuclease-deficient, i.e. it possesses nuclease (preferably endonuclease) activity.

In such embodiments, the CRISPR enzyme may, for example, be a wild-type Cas9 or Cpf1, or a variant or derivative thereof which has endonuclease activity.

Examples of CRISPR enzymes which may be used in this regard include SpCas9, FnCas9, St1Cas9, St3Cas9, NmCas9, SaCas9, AsCpf1, LbCpf1, VQR SpCas9, EQR SpCas9, VRER SpCas9, RHA FnCas9 and KKH SaCas9 (see Komor et al., CRISPR-Based Technologies for the Manipulation of Eukaryotic Genomes, Cell (2017), http://dx.doi.org/10.1016/j.cell.2016.10.044).

In such embodiments, the inducible CRISPR RNA/CRISPR enzyme complex provides an inducible or programmable complex which can be turned “on” at a desired time to target a target DNA and to cleave that target DNA. Such complexes may be used to reduce off-target effects by limiting the active half-life of the complex or by achieving tissue-specific editing in model organisms or in human cells.

In other embodiments, the CRISPR enzyme is an endoribonuclease, e.g. C2c2 or Cas13b, or a variant or derivative thereof.

In some embodiments, the inducible CRISPR RNA/CRISPR enzyme complex comprises one or more functional domains which, when juxtaposed to a target nucleic acid (e.g. a target DNA), promote a desired functional activity, e.g. transcriptional activation of an associated gene. In this case, the aim of the complex is to target the functional domain(s) to the desired target nucleic acid. In some embodiments, the complex may act as a programmable transcription regulator.

Upon binding of the inducible CRISPR RNA to the target nucleic acid, the functional domain is placed in a spatial orientation that allows the functional domain to function in its attributed function.

In some embodiments, one or more functional domains are attached, directly or indirectly, to the inducible CRISPR RNA, preferably to the CRISPR sgRNA.

In some embodiments, one or more functional domains are attached via stem-loop RNA binding proteins (RBPs) to the CRISPR sgRNA.

In other embodiments, one or more functional domains are attached, directly or indirectly, to the CRISPR enzyme.

In some embodiments, the CRISPR sgRNA additionally comprises one or more stem loops to which one or more stem-loop RNA binding proteins (RBPs) are capable of interacting.

These stem loops are in addition to those that are formed between internal regions of the scaffold part of the sgRNA.

Preferably, these one or more stem loops are positioned within the non-spacer element region of the sgRNA, such that the one or more stem loops do not adversely affect the ability of the non-spacer element region of the sgRNA to interact with an endonuclease (e.g. with Cas9), or the ability of the spacer element to hybridise to its target DNA (once it is not bound by the spacer-blocking element).

Examples of suitable stem-loop binding proteins include MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, PRR1 and corn.

The CRISPR sgRNA may therefore additionally comprise one or more stem-loops which are capable of interacting with one or more of the above-mentioned stem-loop binding proteins.

Preferred examples of such stem-loop RNA binding proteins include the bacteriophage MS2 coat proteins (MCPs) which bind to MS2 RNA stem loops; and the PP7 RNA-binding coat protein of the bacteriophage Pseudomonas.

Tagging of RNA stem loops with MS2 coat proteins is a technique based upon the natural interaction of the MS2 protein with a stem-loop structure from the phage genome. It has been used for biochemical purification of RNA-protein complexes and partnered to GFP for detection of RNA in living cells (see, for example, Johansson et al., (1997), “RNA recognition by the MS2 phage coat protein”, Sem. Virol. 8 (3): 176-185).

PP7 RNA-binding coat protein of the bacteriophage Pseudomonas binds a specific RNA sequence and secondary structure. The PP7 RNA-recognition motif is distinct from that of MS2.

The stem-loop RNA binding proteins (RBPs) may themselves be linked to or be capable of interacting with other moieties, e.g. other proteins or polypeptides.

In some embodiments, the stem-loop RNA binding proteins (RBPs) act as adaptor proteins, i.e. intermediaries, which bind both to the stem-loop RNA and to one or more other proteins or polypeptides.

Preferably, the stem-loop RNA binding proteins (RBPs) act as adaptor proteins, i.e. intermediaries, which bind both to the stem-loop RNA and to one or more functional domains.

In some embodiments, the stem-loop RNA binding protein forms a fusion protein with one or more functional domains.

In other embodiments, the one or more functional domains are attached, directly or indirectly, to the CRISPR enzyme.

In some embodiments, the one or more functional domains are attached to the Rec1 domain, the Rec2 domain, the HNH domain, or the PI domain of the SpCas9 protein or any ortholog corresponding to these domains.

In certain embodiments, the one or more functional domains are attached to the Rec1 domain at position 553 or 575; the Rec2 domain at any position of 175-306 or replacement thereof; the HNH domain at any position of 715-901 or replacement thereof; or the PI domain at position 1153 of the SpCas9 protein; or any ortholog corresponding to these domains.

In other embodiments, the dCas9 forms a fusion protein with one or more functional domains.

The functional domain is generally a heterologous domain, i.e. a domain which is not naturally found in the stem-loop RNA binding protein or dCas9.

In some embodiments of the invention, at least one of the one or more functional domains have one or more activities selected from the group consisting of methylase activity, demethylase activity, transcription activation activity, transcription repression activity, transcription release factor activity, histone modification activity, RNA cleavage activity, DNA cleavage activity, nucleic acid binding activity and base-conversion activity.

The functional domain may be an effector domain (e.g. a domain which is capable of stimulating transcription of an associated target gene).

The functional domain is preferably a polypeptide or part thereof, e.g. a domain of a protein which has the desired activity.

In some preferred embodiments, the functional domain has transcription activation activity, i.e. the functional domain acts as a transcriptional activator.

Preferably, one or more of the functional domains is a transcriptional activator which binds to or activates a promoter, thus promoting transcription of the cognate gene.

Examples of transcription factors include heat-shock transcription factors (e.g. HSF1, VP16, VP64, p65 MyoDI and p300).

Transcriptional repression may be achieved by blocking transcriptional initiation (e.g. by targeting the sgRNA to a promoter) or by blocking transcriptional elongation (e.g. by targeting the sgRNA to an exon). It may also be achieved by fusing a repressor domain to the CRISPR enzyme which induces heterochromatization (e.g. the KRAB domain).

Examples of transcriptional repressor domains include KRAB domain, a SID domain and a SID4X domain.

In a further embodiment, the invention provides a composition comprising:
(a) an inducible CRISPR RNA of the invention; and
(b) a CRISPR enzyme.

In a further embodiment, the invention provides a kit comprising:
(a) an inducible CRISPR RNA of the invention; and
(b) a CRISPR enzyme,
   in a form suitable for sequential, separate or simultaneous use.

The use may be a method of the invention.

The invention also provides a DNA molecule encoding an inducible CRISPR RNA of the invention. The DNA molecule may additionally encode a CRISPR enzyme.

The invention also provides a vector encoding a DNA molecule of the invention.

In a further embodiment, the invention provides a composition comprising:
(a) a DNA molecule encoding an inducible CRISPR RNA of the invention; and
(b) a DNA molecule encoding a CRISPR enzyme.

In a further embodiment, the invention provides a kit comprising:
(a) a vector encoding an inducible CRISPR RNA of the invention; and
(b) a vector encoding a CRISPR enzyme;
   in a form suitable for sequential, separate or simultaneous use.

The use may be a method of the invention.

In a further embodiment, the invention provides a method for inducibly targeting a CRISPR complex to a target DNA in a host cell, the method comprising the steps:
(i) expressing in the host cell:
   (a) a first inducible CRISPR RNA of the invention, wherein the nucleotide sequence of the spacer element is fully or partially complementary to a region of the first target DNA; and
   (b) a CRISPR enzyme,
such that the first inducible CRISPR RNA and CRISPR enzyme form a CRISPR complex; and
(ii) inducing, at a desired time, cleavage of the cleavable loop element of the first inducible CRISPR RNA,
   thus allowing the spacer element to target the CRISPR complex to the first target DNA.

In some embodiments, the method additionally comprises the steps:
(i) expressing in the host cell:
   (a) a second inducible CRISPR RNA of the invention, wherein the nucleotide sequence of the spacer element is fully or partially complementary to a region of a second target DNA; and
   (b) a CRISPR enzyme,
such that the second inducible CRISPR RNA and CRISPR enzyme form a second CRISPR complex; and
(ii) inducing, at a desired time, cleavage of the cleavable loop element of the second inducible CRISPR RNA,
   thus allowing the spacer element to target the second CRISPR complex to the second target DNA.

In some embodiments, the method additionally comprises the steps:
(i) expressing in the host cell:
   (a) a plurality of inducible CRISPR RNAs of the invention, wherein the nucleotide sequences of the spacer elements are independently fully or partially complementary to regions of the plurality of target DNAs; and
   (b) a CRISPR enzyme,
such that the plurality of inducible CRISPR RNAs and CRISPR enzymes form a plurality of CRISPR complexes; and
(ii) inducing, at a desired time, cleavage of the cleavable loop elements of the plurality of inducible CRISPR RNAs,
   thus allowing the spacer elements to target the plurality of CRISPR complexes to the plurality of target DNAs.

As used herein, the term “plurality” includes 2, 3, 4, 5, 6, 7, 8, 9, 10 or more.

In some embodiments, cleavage of the cleavable loop elements of the first and second inducible CRISPR RNAs of the invention is inducible by the same inducer (preferably wherein the first and second inducible CRISPR RNAs (independently) comprise the same cleavable loop element).

In other embodiments, cleavage of the cleavable loop elements of the first and second inducible CRISPR RNAs of the invention is inducible by different inducers (preferably wherein the first and second inducible CRISPR RNAs comprise different cleavable loop elements).

The invention also provides methods utilising third, fourth, fifth, etc. inducible CRISPR RNAs of the invention, mutatis mutandis.

In a further embodiment, the invention provides a method for inducibly targeting a functional domain to a target DNA in a host cell, the method comprising the steps:
(i) expressing in the host cell:
   (a) an inducible CRISPR RNA of the invention, wherein the nucleotide sequence of the spacer element is fully or partially complementary to a region of the target DNA; and
   (b) a CRISPR enzyme,
such that the inducible CRISPR RNA and CRISPR enzyme form a CRISPR complex, wherein the CRISPR complex comprises one or more functional domains; and
(ii) inducing, at a desired time, cleavage of the cleavable loop element of the inducible CRISPR RNA,
   thus allowing the spacer element to direct binding of the CRISPR complex to the target DNA and thus targeting the one or more functional domains to the target DNA.

The method provides a method for inducibly targeting a functional domain to a target DNA in a host cell.

As used herein, the term “inducibly” or “inducible” refers to the ability to induce cleavage of the cleavable loop element of the inducible CRISPR RNA at a desired time, thus allowing the spacer element to direct binding of the CRISPR complex to the target DNA at the desired time.

The host cells may be any host cells in which it is desired to perform the method. The host cells may, for example, be prokaryotic cells or eukaryotic cells, preferably eukaryotic cells. In some embodiments, the host cells are mammalian cells, preferably human cells.

In some embodiments, the host cells are microencapsulated cells. Micro-encapsulation is a process whereby a genetically-modified cell is encapsulated before delivery inside a living organism. This aims to seal the engineered cells in order to protect them from the host immune system and enable straightforward removal after completion of the therapy (e.g. Auslander S. et al., 2012. “Smart medication through combination of synthetic biology and cell microencapsulation”, Metab. Eng. 14: 252-260).

The inducible CRISPR RNA and CRISPR enzymes are both expressed within the host cell. The expression may be in any order.

For example, one or more expression vectors comprising the inducible CRISPR RNA and CRISPR enzymes may be transfected into the host cells.

In some embodiments, an expression vector comprising a DNA sequence coding for the inducible CRISPR RNA is transfected into the host cells first and then an expression vector comprising a DNA sequence coding for the CRISPR enzyme is transfected into the host cells.

In other embodiments, an expression vector comprising a DNA sequence coding for the CRISPR enzyme is transfected into the host cells first and then an expression vector comprising a DNA sequence coding for the inducible CRISPR RNA is transfected into the host cells.

In yet other embodiments, expression vectors comprising a DNA sequence coding for the CRISPR enzyme and an expression vector comprising a DNA sequence coding for the inducible CRISPR RNA are transfected simultaneously into the host cells.

Preferably, a single (type of) expression vector comprising a DNA sequence coding for the CRISPR enzyme and a DNA sequence coding for the inducible CRISPR RNA is transfected into the host cells.

In other embodiments, the host cells are ones which endogenously express the CRISPR enzyme within the host cells.

The CRISPR complex preferably comprises one or more functional domains. These functional domains may be attached via the sgRNA or via the CRISPR enzyme.

In embodiments of the invention which refer to a first and second (or more) CRISPR complex, the CRISPR enzymes in the first and second (and more) CRISPR complexes may be the same or different. Preferably, the CRISPR enzymes in each CRISPR complex are the same.

At a desired time, the cleavage of the cleavable loop in the inducible CRISPR RNA may be induced.

The form of the induction will depend on the structure and composition of the cleavable loop.

In some embodiments of the methods of the invention, the host cell comprises two or more (e.g. 2, 3, 4, 5, 6, 7, 8, 9 or 10) different inducible CRISPR RNAs of the invention, wherein the nucleotide sequences of the spacer elements are independently fully or partially complementary to regions of two or more different target DNAs.

In some embodiments, cleavage of the cleavable loop elements of the two or more (e.g. 2, 3, 4, 5, 6, 7, 8, 9 or 10) different inducible CRISPR RNAs of the invention is inducible by the same inducer (e.g. the same cleaving moiety). For example, each different inducible CRISPR RNA of the invention may (independently) share a common cleavable loop element.

In other embodiments, cleavage of the cleavable loop elements of the two or more (e.g. 2, 3, 4, 5, 6, 7, 8, 9 or 10) different inducible CRISPR RNAs of the invention may be inducible by different inducers (e.g. different cleaving moieties). For example, each different inducible CRISPR RNA of the invention may comprise a different cleavable loop element.

The one or more functional domains which may be comprised within the CRISPR complex may have one or more activities selected from the group consisting of methylase activity, demethylase activity, transcription activation activity, transcription repression activity, transcription release factor activity, histone modification activity, RNA cleavage activity, DNA cleavage activity and nucleic acid binding activity.

Hence the inducible CRISPR RNAs and methods of the invention may be used, for example, for one or more of the following: genome editing, epigenetic alteration, base editing, DNA labelling, base-conversion and lineage tracing throughout development or in disease states.

The invention also provides a method for inducible editing of a target gene in a target DNA in a host cell, the method comprising the steps:
(i) expressing in the host cell:
   (a) an inducible CRISPR RNA of the invention, wherein the nucleotide sequence of the spacer element is fully or partially complementary to a region of the target gene; and
   (b) a CRISPR enzyme with catalytic activity,
such that the inducible CRISPR RNA and CRISPR enzyme form a CRISPR complex; and
(ii) inducing, at a desired time, cleavage of the cleavable loop element of the inducible CRISPR RNA,
   thus allowing the spacer element to direct binding of the CRISPR complex to the target gene and thus inducing editing of the target gene.

Preferably, the CRISPR enzyme is one which has endonuclease activity.

The term “editing” includes cleavage of the target gene; this may be required for downstream editing applications, e.g. NHEJ, Homology directed repair with donor template, etc.

The invention also provides a method for inducing epigenetic modification of a target DNA in a host cell, the method comprising the steps:
(i) expressing in the host cell:
   (a) an inducible CRISPR RNA of the invention, wherein the nucleotide sequence of the spacer element is fully or partially complementary to a region of the target DNA; and
   (b) a catalytically-inactive CRISPR enzyme,
such that the inducible CRISPR RNA and CRISPR enzyme form a CRISPR complex, wherein the CRISPR complex comprises one or more domains which are capable of epigenetic modification of the target DNA; and
(ii) inducing, at a desired time, cleavage of the cleavable loop element of the inducible CRISPR RNA,
   thus allowing the spacer element to direct binding of the CRISPR complex to the target DNA and thus targeting the one or more domains which are capable of epigenetic modification of the target DNA to the region of the target DNA, thus inducing epigenetic modification of the target DNA.

Preferably, the CRISPR enzyme is catalytically-inactive Cas9, e.g. dCas9.

Preferably, the one or more domains which are capable of epigenetic modification have methylase activity.

The invention also provides a method for inducible editing of one or more nucleotides of a target DNA in a host cell, the method comprising the steps:
(i) expressing in the host cell:
   (a) an inducible CRISPR RNA of the invention, wherein the nucleotide sequence of the spacer element is fully or partially complementary to a region of the target DNA; and
   (b) a catalytically-inactive CRISPR enzyme,
such that the inducible CRISPR RNA and CRISPR enzyme form a CRISPR complex, and wherein the CRISPR complex comprises one or more effector domains which have nucleotide-editing properties; and
(ii) inducing, at a desired time, cleavage of the cleavable loop element of the inducible CRISPR RNA,
   thus allowing the spacer element to direct binding of the CRISPR complex to the target DNA and thus targeting the one or more effector domains which have nucleotide-editing properties to the region of the target DNA, thus editing one of more nucleotides of the target DNA.

Preferably, the CRISPR enzyme is catalytically-inactive Cas9, e.g. dCas9.

Preferably, the effector domain has cytidine deaminase activity.

These so-called second-generation CRISPR ‘base editors’ use catalytically-modified Cas9 (dCas9) fused to a cytidine deaminase enzyme encoded by the human APOBEC1 gene or the sea lamprey PmCDA1 gene. Importantly, this fusion complex is still guided by RNA, but it does not cause double strand breaks at the target site. Instead, the cytidine deaminase converts cytosine bases into uridines, which are then repaired by error-prone mechanisms that result in various point mutations. The system also enables more specific and desired point mutations, such as C-T or G-A transitions when the uracil-DNA glycosylase pathway is inhibited. (See Kuscu, C., & Adli, M. (2016). “CRISPR-Cas9-AID base editor is a powerful gain-of-function screening tool”. Nature Methods, 13(12), 983-984. http://doi.org/10.1038/nmeth.4076).

More preferably, therefore, the CRISPR enzyme is dCas9 with a tethered cytidine deaminase enzyme (e.g. Komor, A. C. et al. (2016). “Programmable editing of a target base in genomic DNA without double-stranded DNA cleavage”. Nature, 533(7603), 420-424; Nishida, K., et al. (2016). “Targeted nucleotide editing using hybrid prokaryotic and vertebrate adaptive immune systems”. Science (New York, N.Y.) aaf8729. http://doi.org/10.1126/science.aaf8729).

The invention also provides a method for inducible labelling of a target DNA in a host cell, the method comprising the steps:
(i) expressing in the host cell:
   (a) an inducible CRISPR RNA of the invention, wherein the nucleotide sequence of the spacer element is fully or partially complementary to a region of the target DNA; and
   (b) a catalytically-inactive CRISPR enzyme,
such that the inducible CRISPR RNA and CRISPR enzyme form a CRISPR complex, and wherein the CRISPR complex comprises one or more labelled domains; and
(ii) inducing, at a desired time, cleavage of the cleavable loop element of the inducible CRISPR RNA,
   thus allowing the spacer element to direct binding of the CRISPR complex to the target DNA and thus targeting the one or more labelled domains to the region of the target DNA, thus labelling the target DNA.

Preferably, the CRISPR enzyme is catalytically-inactive Cas9, e.g. dCas9.

Preferably, the labelled domain is a radioactive or fluorescent polypeptide (e.g. green fluorescent protein, GFP). For example, see Ma, H. et al., (2016). “Multiplexed labeling of genomic loci with dCas9 and engineered sgRNAs using CRISPRainbow”. Nature Biotechnology, 34(5), 528-530. http://doi.org/10.1038/nbt.3526.

More preferably, therefore, the CRISPR enzyme is dCas9 which is labelled with GFP.

In other embodiments, the invention may be used to trace the lineage of cells within a multicellular organism. This provides a way to encode within the cell a record of cell divisions in order to be able to trace back the origin of a particular cell and to help to understand cell division, differentiation and migration. This may be achieved by encoding within the DNA of a mother cell a locus which is going to be mutated at each division. This provides a genetic barcode system which helps lineage tracing.

In one aspect, the barcode locus is made of repeats of a target DNA sequence which can be targeted by a sgRNA. Throughout the lifespan of a cell, the locus accumulates mutations which are transmitted to the daughter cells (e.g. McKenna, A. et al. (2016). “Whole organism lineage tracing by combinatorial and cumulative genome editing”. Science (New York, N.Y.), aaf7907. http://doi.org/10.1126/science.aaf7907).

In a second aspect, the barcode locus encodes a sgRNA (modified to accommodate a NGG PAM in the scaffold sequence) which targets itself. When transcribed upon induction, the sgRNA targets its own locus, thus creating a new sgRNA (e.g. Kalhor, R. et al. (2016). “Rapidly evolving homing CRISPR barcodes”. Nature Methods. http://doi.org/10.1038/nmeth.4108); and Perli, S. D. et al. (2016). “Continuous genetic recording with self-targeting CRISPR-Cas in human cells”. Science (New York, N.Y.), aag0511. http://doi.org/10.1126/science.aag0511).

The invention therefore also provides a method for lineage tracing of daughter cells derived from a host cell, wherein the host cell comprises a first genetic barcode comprising a plurality of repeats of a first target DNA, the method comprising the steps:
(i) expressing in the host cell:
   (a) a first inducible CRISPR RNA of the invention, wherein the nucleotide sequence of the spacer element is fully or partially complementary to a region of a first target DNA in the host cell; and
   (b) a catalytically-active CRISPR enzyme,
such that the first inducible CRISPR RNA and CRISPR enzyme form a CRISPR complex; and
(ii) inducing, at a desired time, cleavage of the cleavable loop element of the first inducible CRISPR RNA,
   thus allowing the spacer element to direct binding of the CRISPR complex to the first target DNA and wherein the CRISPR enzyme produces one or more mutations in the first genetic barcode which are transmitted to daughter cells, and which mutations can be used to characterise the lineage of the daughter cells.

In some embodiments, the method additionally comprises the steps of:
(i) expressing in the host cell:
   (a) a second inducible CRISPR RNA of the invention, wherein the nucleotide sequence of the spacer element is fully or partially complementary to a region of a second target DNA in the host cell, wherein the second target DNA forms part of a second genetic barcode comprising a plurality of repeats of the second target DNA; and
   (b) a catalytically-active CRISPR enzyme,
such that the second inducible CRISPR RNA and CRISPR enzyme form a second CRISPR complex; and
(ii) inducing, at a desired time, cleavage of the cleavable loop element of the second inducible CRISPR RNA,
   thus allowing the spacer element to direct binding of the second CRISPR complex to the second target DNA and wherein the CRISPR enzyme produces one or more mutations in the second genetic barcode which are transmitted to daughter cells, and which mutations can be used to characterise the lineage of the daughter cells.

Additional (e.g. third, fourth, etc.) genetic barcodes and inducible CRISPR RNAs of the invention may be utilised, mutatis mutandis.

In some preferred embodiments, cleavage of the cleavable loop element of one or more of the inducible CRISPR RNAs is under control of a tissue-specific promoter (e.g. a brain-specific promoter). For example, expression of Csy4 in the cell may be placed under the control of a tissue-specific (e.g. brain) promoter.

In some embodiments, the method additionally includes the step of sequencing part or all of one or more of the genetic barcodes.

In some embodiments, the genetic barcode comprises 9-12 repeats of a target DNA.

The genetic barcode is integrated into the host cell genome.

A method for lineage tracing of daughter cells derived from a host cell, the method comprising the steps:
(i) expressing in the host cell:
   (a) a first inducible CRISPR RNA of the invention from the genome of the host cell, wherein the CRISPR sgRNA is associated with a PAM sequence; and
   (b) a catalytically-active CRISPR enzyme,
such that the first inducible CRISPR RNA and CRISPR enzyme form a first CRISPR complex; and
(ii) inducing, at a desired time, cleavage of the cleavable loop element of the first inducible CRISPR RNA,
   thus allowing the spacer element to direct binding of the first CRISPR complex to the genomic DNA which encodes the first CRISPR sgRNA and wherein the CRISPR enzyme produces one or more mutations in the genomic DNA which encodes the first CRISPR sgRNA which mutations are transmitted to daughter cells, and which mutations can be used to characterise the lineage of the daughter cells.

In some embodiments, the method additionally comprises the steps of:
(i) expressing in the host cell:
   (a) a second inducible CRISPR RNA of the invention from the genome of the host cell, wherein the CRISPR sgRNA is associated with a PAM sequence; and
   (b) a catalytically-active CRISPR enzyme,
such that the second inducible CRISPR RNA and CRISPR enzyme form a second CRISPR complex; and
(ii) inducing, at a desired time, cleavage of the cleavable loop element of the second inducible CRISPR RNA,
   thus allowing the spacer element to direct binding of the second CRISPR complex to the genomic DNA which encodes the second CRISPR sgRNA and wherein the CRISPR enzyme produces one or more mutations in the genomic DNA which encodes the second CRISPR sgRNA which mutations are transmitted to daughter cells, and which mutations can be used to characterise the lineage of the daughter cells.

Additional (e.g. third, fourth, etc.) inducible CRISPR RNAs of the invention may be utilised, mutatis mutandis.

In some preferred embodiments, cleavage of the cleavable loop element of one or more of the inducible CRISPR RNAs is under control of a tissue-specific promoter (e.g. a brain-specific promoter). For example, expression of Csy4 in the cell may be placed under the control of a tissue-specific (e.g. brain) promoter.

The invention also provides a method for inducing transcription of a target gene in a target DNA in a host cell, the method comprising the steps:
(i) expressing in the host cell:
   (a) an inducible CRISPR RNA of the invention, wherein the nucleotide sequence of the spacer element is fully or partially complementary to a region of the target DNA in the vicinity of the target gene; and
   (b) a catalytically-inactive CRISPR enzyme,
such that the inducible CRISPR RNA and CRISPR enzyme form a CRISPR complex, wherein the CRISPR complex comprises one or more effector domains; and
(ii) inducing, at a desired time, cleavage of the cleavable loop element of the inducible CRISPR RNA,
   thus allowing the spacer element to direct binding of the CRISPR complex to the target DNA and thus targeting the one or more effector domains to the region of the target DNA in the vicinity of the target gene, thus inducing transcription of the target gene.

The invention also provides a method for inducing coordinated transcription of two or more target genes in one or more target DNAs in a host cell, the method comprising the steps:
(i) expressing in the host cell:
   (a) an inducible CRISPR RNA of the invention, wherein the nucleotide sequence of the spacer element is independently fully or partially complementary to a region of one or more target DNAs in the vicinity of the two or more target genes; and
   (b) a catalytically-inactive CRISPR enzyme,
such that the inducible CRISPR RNA and CRISPR enzyme form a CRISPR complex, wherein the CRISPR complex comprises one or more effector domains; and
(ii) inducing, at a desired time, cleavage of the cleavable loop element of the inducible CRISPR RNA,
   thus allowing the spacer element to direct binding of the CRISPR complex to the two or more target DNAs and thus targeting the one or more effector domains to the regions of the target DNAs in the vicinity of the target genes, thus inducing coordinated transcription of the two or more target genes.

The invention also provides a method for inducing coordinated transcription of two or more target genes in one or more target DNAs in a host cell, the method comprising the steps:
(i) expressing in the host cell:
   (a) two or more different inducible CRISPR RNAs of the invention, wherein the nucleotide sequences of the spacer elements are independently fully or partially complementary to regions of one or more target DNAs in the vicinity of the two or more different target genes, and wherein the cleavable loop elements of the two or more different inducible CRISPR RNAs of the invention are cleavable by the same inducer; and
   (b) a catalytically-inactive CRISPR enzyme,
such that the different inducible CRISPR RNAs and the CRISPR enzyme form different CRISPR complexes, wherein the CRISPR complexes comprise one or more effector domains; and
(ii) inducing, at desired time, cleavage of the cleavable loop elements of the inducible CRISPR RNAs,
   thus allowing the spacer elements to direct binding of the CRISPR complexes to the two or more target DNAs at the desired time and thus targeting the one or more effector domains to the regions of the target DNAs in the vicinity of the target genes, thus inducing coordinated transcription of the two or more target genes.

The “inducer” is the entity which cleaves the cleavable loop element. Preferably, the two or more different inducible CRISPR RNAs of the invention (independently) comprise the same cleavable loop element.

The invention also provides a method for inducing orthogonal transcription of two or more target genes in one or more target DNAs in a host cell, the method comprising the steps:
(i) expressing in the host cell:
   (a) two or more different inducible CRISPR RNAs of the invention, wherein the nucleotide sequences of the spacer elements are independently fully or partially complementary to regions of one or more target DNAs in the vicinity of the two or more different target genes; and
   (b) a catalytically-inactive CRISPR enzyme,
such that the different inducible CRISPR RNAs and the CRISPR enzyme form different CRISPR complexes, wherein the CRISPR complexes comprise one or more effector domains; and
(ii) inducing, at desired times, cleavage of the cleavable loop elements of the inducible CRISPR RNAs,
   thus allowing the spacer elements to direct binding of the CRISPR complexes to the two or more target DNAs at the desired times and thus targeting the one or more effector domains to the regions of the target DNAs in the vicinity of the target genes, thus inducing orthogonal transcription of the two or more target genes.

Preferably, upon induction, the cleavable loops are cleaved independently (e.g. they are cleaved by different enzymes).

In some preferred embodiments, the cleavable loops are independently cleaved by Csy4 and Cas6A.

In another embodiment, the invention provides a method for detecting the presence of a miRNA in a test sample, the method comprising the steps:

(i) contacting a CRISPR complex with the test sample and a reporter DNA,
wherein the CRISPR complex comprises
   (a) an inducible CRISPR RNA of the invention,
   wherein the nucleotide sequence of the spacer element is fully or partially complementary to a region of the reporter DNA;
   and wherein the cleavable loop element comprises a miRNA response element (MRE) which is capable of being bound by the miRNA; and
   (b) a CRISPR enzyme,
      under conditions such that if the miRNA is present in the test sample, the miRNA will bind to the MRE in the cleavable loop element thus inducing cleavage of the cleavable loop element, thus allowing the spacer element to bind to the region of the reporter DNA, and
      (ii) detecting the presence or absence of the reporter gene or reporter gene product, this being indicative of the presence or absence of the miRNA in the test sample.

The invention also provides a method for detecting the presence of one or more miRNAs in a test sample, the method comprising the steps:

(i) contacting a plurality of CRISPR complexes with the test sample and one or more reporter DNAs, wherein the CRISPR complexes each independently comprise
(a) an inducible CRISPR RNA of the invention, wherein the nucleotide sequence of the spacer element is fully or partially complementary to a region of the one or more reporter DNAs; and wherein the cleavable loop element comprises a miRNA response element (MRE) which is capable of being bound by one of the miRNAs; and
(b) a CRISPR enzyme,
   under conditions such that if one or more of the miRNAs are present in the test sample, those miRNAs will independently bind to a cognate MRE in a cleavable loop element thus inducing cleavage of that cleavable loop element, thus allowing the spacer element to bind to the region of the one or more reporter DNAs, and
   (ii) detecting the presence or absence of the one or more reporter genes or one or more reporter gene products, this being indicative of the presence or absence of one or more of the miRNAs in the test sample.

Preferably, the miRNA is present in the form of a miRISC complex which is capable of cleaving a cognate MRE.

In some embodiments, the CRISPR enzyme is a catalytically-active CRISPR enzyme which cleaves the reporter DNA. In such cases, the absence of or cleavage of the reporter gene is indicative of the presence of the miRNA in the test sample.

In other embodiments, the CRISPR enzyme is catalytically-inactive. In such cases, the CRISPR complex may comprise one or more functional domains (e.g. which promote transcription of the reporter DNA, e.g. VP64). In such cases, the presence of a reporter gene product (e.g. mRNA or polypeptide) is indicative of the presence of the miRNA in the test sample.

The transcription of the reporter gene may be detected in any suitable means, e.g. by detection of the mRNA (e.g. by PCR-based methods) or by detection of the translation product (e.g. wherein the translation product is an assayable polypeptide, e.g. a fluorescent polypeptide).

In some embodiments, the test sample is a sample of blood or plasma, or other patient-derived fluid.

In some preferred embodiments, the miRNA is one which is expressed (e.g. under-expressed or over-expressed) in association with a disease or disorder. Preferably, the disease is cancer.

The nucleotide sequence of the spacer element is fully or partially complementary to a region of the target DNA in the vicinity of the target gene. As used herein, the term “vicinity” refers to a distance such that, upon binding of the spacer element to the region of the target DNA, the one or more effector domains which are attached to the CRISPR complex (either via the sgRNA or via the CRISPR enzyme) are placed in a spatial orientation which allows them to activate transcription of the target gene.

For example, the effector domains may be placed in a position which allows them to bind to a promoter or enhancer element, thus activating or stimulating transcription of the associated gene.

In some embodiments, the nucleotide sequence of the spacer element is fully or partially complementary to a region of the target DNA which is within 200 kb (preferably within 100 kb, 50 kb, 20 kb, 10 kb, 5 kb, 1 kb, 500 bases, 200 bases, 100 bases or 20 bases) of a regulatory element associated with the target gene. Preferably, the regulatory element is an enhancer element or a promoter element.

In some embodiments, the nucleotide sequence of the spacer element is fully or partially complementary to a region of the target DNA which allows the activation of a control element, preferably activation of a promoter element, more preferably activation of an element, which is activated by the binding of a VP64, p65, MyoD or HSF1 activation domain. There may, for example, be 1, 2, 3, 4, 5, 6, 7, 8, 9, 10 or more target genes. There may, for example, be 1, 2, 3, 4, 5, 6, 7, 8, 9 10 or more target DNAs.

The target DNAs may be adjacent regions within a single gene or control element. For example, one or more target DNAs may be adjacent regions within the promoter of a gene.

As used herein, the term “orthogonal” means independent, i.e. the two or more target genes may be independently regulated or independently transcribed.

Cleavage of the cleavable loop elements of the CRISPR sgRNA may be induced at a desired time.

For example, a genetically-coded endoribonuclease may be activated within the host cells. A vector or plasmid encoding the endoribonuclease may be transfected into the cell at a desired time. One or more endoribonucleases may be under the control of one or more independent promoters. One or more of the promoters may be activated at desired times.

In other examples, an antisense oligonucleotide whose sequence is fully or partially complementary to the cleavable loop may be produced within the host cell or introduced into the host cell. Antisense oligonucleotides may be transfected into cells using polyethyleneimine (PEI) or other known transfection methods.

In the embodiments of the invention wherein the cleavable loop element comprises a miRNA response element (MRE), a miRNA which is capable of binding to the MRE may be produced within the host cell or introduced into the host cell. Preferably, the nucleotide sequence of the miRNA is fully-complementary to the nucleotide sequence of the MRE.

Preferably, the miRNA is present in the form of a miRISC complex, which targets the MRE and cleaves the cleavable loop element.

In yet other examples, cleavage of an allosteric self-cleaving hammerhead ribozyme may be induced by introducing its cognate ligand (which promotes a change in conformation of the ribozyme (strand displacement) and which allows the ribozyme to resume its catalytic activity). Cleavage of the ribozyme facilitates dissociation of the spacer-blocking element from the spacer element, rendering the sgRNA competent to target the target DNA. In some embodiments, the cognate ligand is theophylline.

In yet a further embodiment, the invention provides an in vivo method of inducing transcription of a target gene in a subject, the method comprising the steps:
(i) expressing an inducible CRISPR RNA of the invention and a catalytically-inactive CRISPR enzyme in a host cell, such that the inducible CRISPR RNA and CRISPR enzyme form a CRISPR complex, wherein the CRISPR complex comprises one or more effector domains, and wherein the spacer-blocking element is bound to the spacer element;
(ii) introducing the host cell into a subject; and
(iii) introducing into the subject an agent which cleaves the cleavable loop element,
   thus allowing the spacer element to direct binding of the CRISPR complex to a target DNA in the subject which is in the vicinity of the target gene and thus targeting the one or more effector domains to the region of the target DNA in the vicinity of the target gene, thus inducing transcription of the target gene in the subject.

Preferably the host cells are microencapsulated cells.

In some embodiments, the subject is a eukaryote (e.g. zebrafish, Drosophila, mouse), more preferably a mammal (e.g. mouse, human).

The invention also provides a sensor device configured to carry out a method of the invention.

The disclosure of each reference set forth herein is specifically incorporated herein by reference in its entirety.

### BRIEF DESCRIPTION OF THE FIGURES

FIG. 1: Inhibition of CRISPR-TA activity by SBH-sgRNAs.

(a) Conceptual framework underlying the design of inducible sgRNAs for modulation of target gene output. In CRISPR transcription activator (CRISPR-TA)-based studies, the transcriptional rate of a gene of interest is elevated by anchoring at TSS an effector-tethered sgRNA-dCas9 complex (left). Appending a back-fold extension to the 5′ end of the native sgRNA promotes the formation of a spacer blocking hairpin (SBH) expected to switch the sgRNA to a quiescent state (OFF-state) (middle). Replacing the basic loop with conditional RNA cleaving units enables generation of inducible SBH designs (iSBH), which can restore CRISPR-TA activity in the presence of specific inducers (spacer release) (right). (b) HEK-293T cells were co-transfected with plasmids containing dCas9-VP64, either 8×CTS1-mCMVp-EYFP or 8×CTS2-mCMVp-ECFP and the following sgRNAs: nv-SCR (native sgRNA with scramble spacer sequence); nv-CTS1 and nv-CTS2 (native sgRNA targeting CTS1 and CTS2 respectively); SBH⁽⁰⁾CTS1 and SBH⁽⁰⁾CTS2 (SBH-sgRNAs with full spacer coverage); SBH^{(ctrl-1)}CTS1 and SBH^{(ctr1-1)}CTS2 (control SBH-sgRNAs with offset 5′ end hairpin structures and accessible spacers). Flow cytometric analysis (48 h post-transfection) revealed complete SBH-mediated inhibition of CRISPR-TA activity relative to native and control sgRNAs (see also FIG. 3b). Representative flow cytometry scatter plots show reporter activation (EYFP, ECFP channels) plotted against sgRNA transfection (iBlue channel) for CTS1 and CTS2 spacers. (c) Sequence and secondary structure of prototype SBHs designed to silence two sgRNAs (CTS1 and CST2 spacers). Superscript annotation (⁽⁰⁾) denotes the number of spacer free nucleotides. +G1(U6) refers to the G nucleotide required for U6 transcription. (d-f) Consequence of back-fold extension length on SBH-mediated inhibition of CRISPR-TA activity. RNA secondary structure of SBH variants with a progressive increase in the number free spacer nucleotides (SBH^{(0,5,10,15)}CTS2) (d). Representative flow cytometry scatter plots showing the impact of incremental spacer release (SBH^{(5,10,15)}CTS2) on reporter gene activation (axes represent reporter expression against sgRNA plasmid transfection) (e). Quantification of ECFP reporter fluorescence from matching experiments using the following sgRNAs: nv-SCR, nv-CTS2, SBH⁽⁵⁾CTS2, SBH⁽¹⁰⁾CTS2, SBH⁽¹⁵⁾CTS2 (n=3, mean+/−SD, a.u. arbitrary units) (f).

FIG. 2: Schematic of CRISPR-TA reporter assay in HEK293-T cells.

A bicistronic vector is used to couple the expression of U6-driven sgRNAs with a fluorescent reporter (iBlue). The dCas9-VP64 gene is expressed under a CMV promoter from a separate plasmid. The assembled sgRNA-dCas9-VP64 complex is targeted to the synthetic enhancer (8×CRISPR target sites) driving the expression of a target gene (XFP), by programming the sgRNA spacer sequence. In this study two sgRNA spacers were programmed (CTS1, CTS2), to drive specific expression (with no detectable crosstalk) of two corresponding reporter target genes (8×CTS1-mCMVp-EYFP-pA and 8×CTS2-mCMVp-ECFP-pA respectively).

FIG. 3: Control SBH-sgRNA mimics.

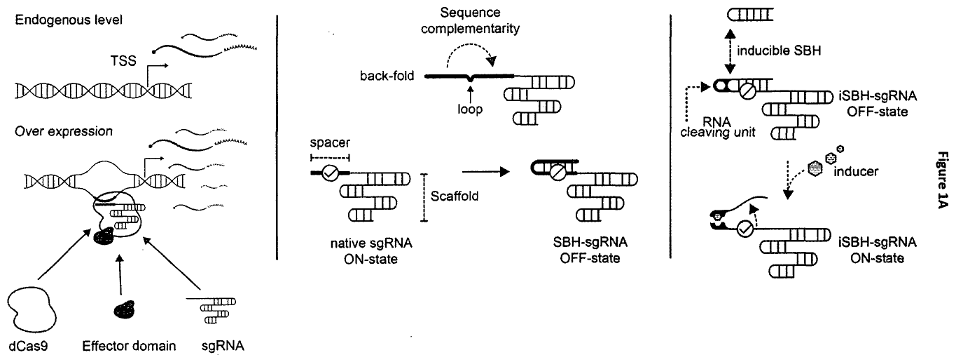

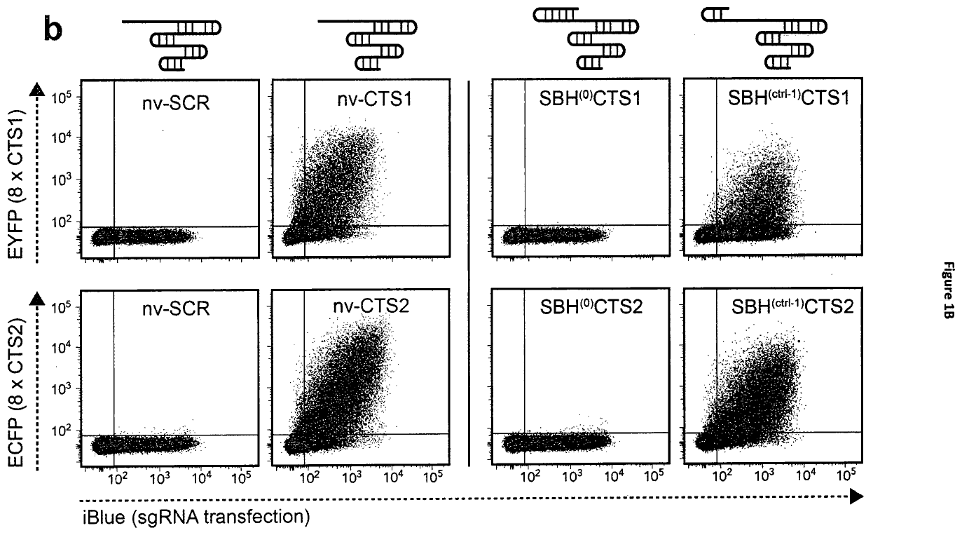

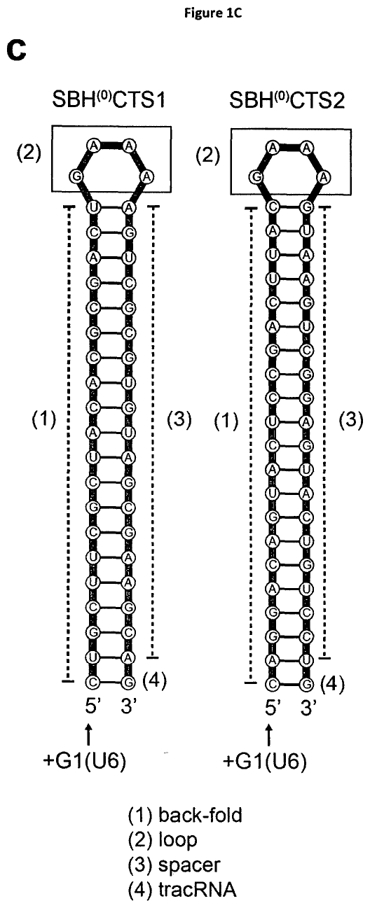

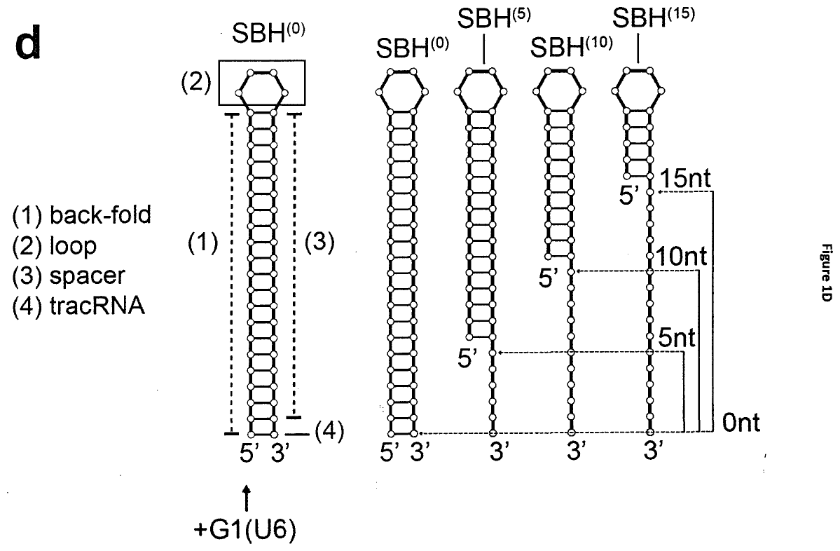

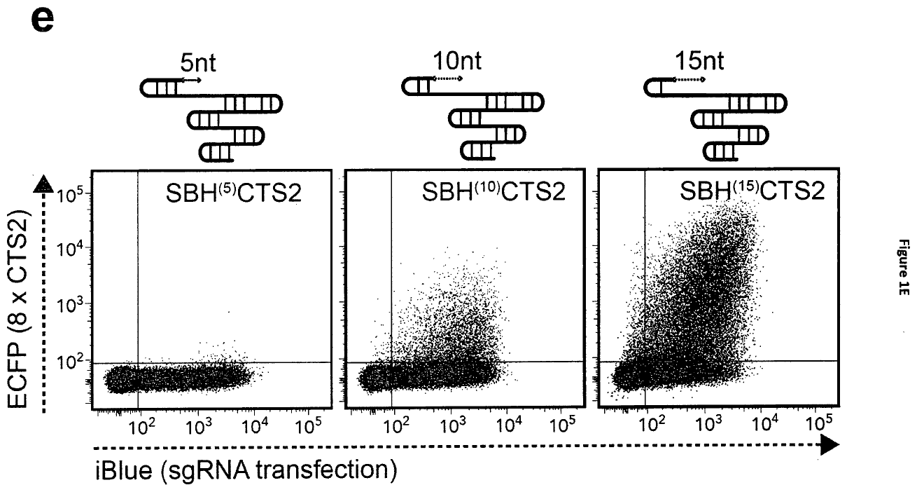

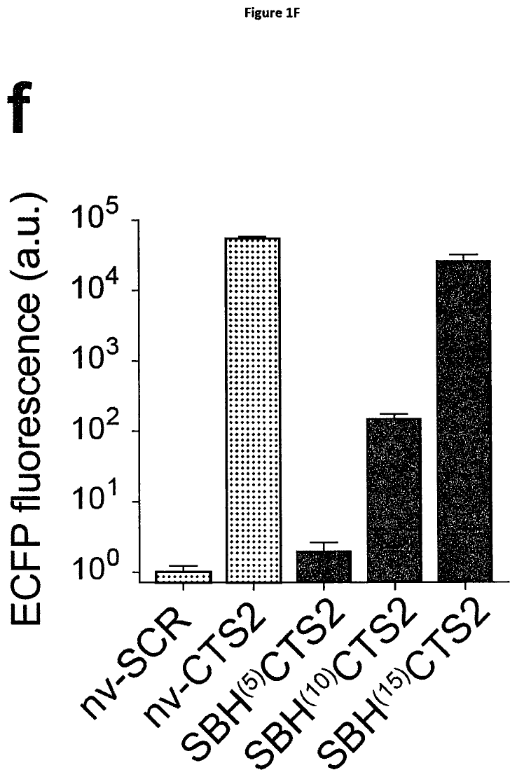

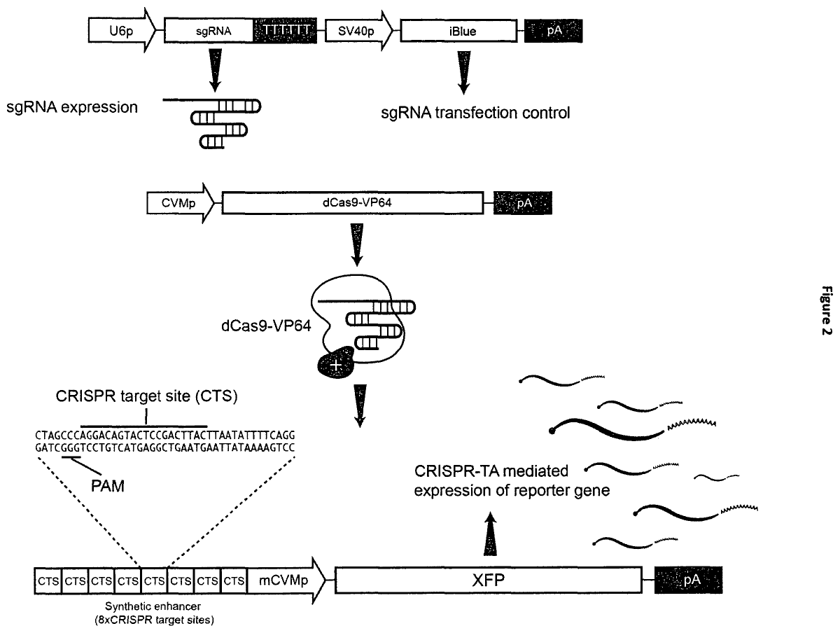

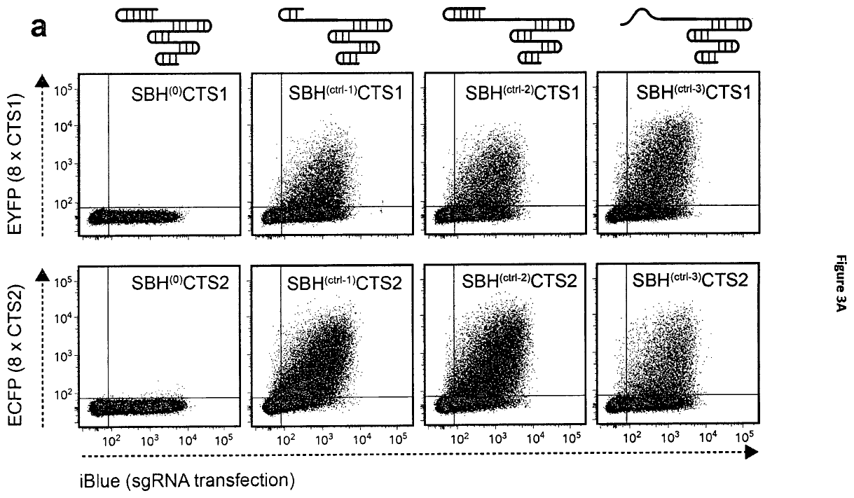

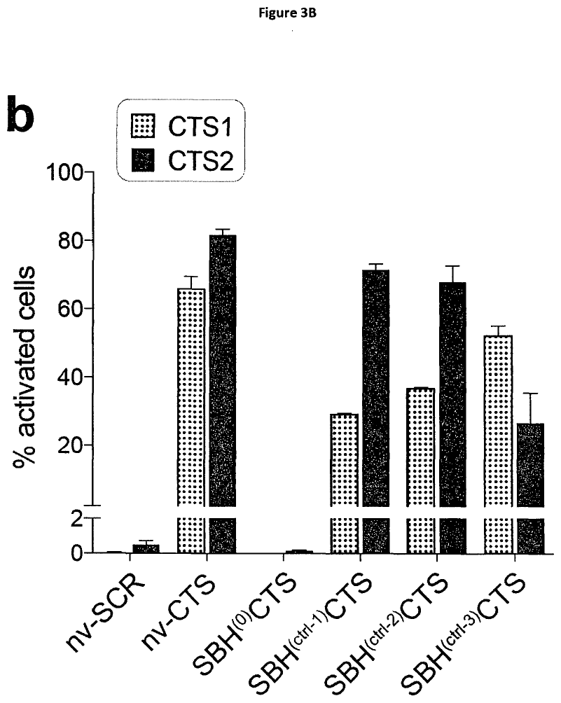

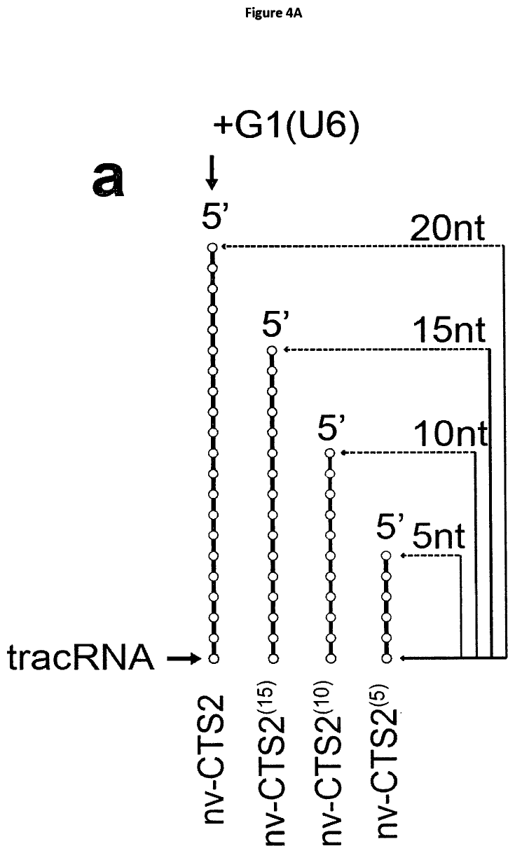

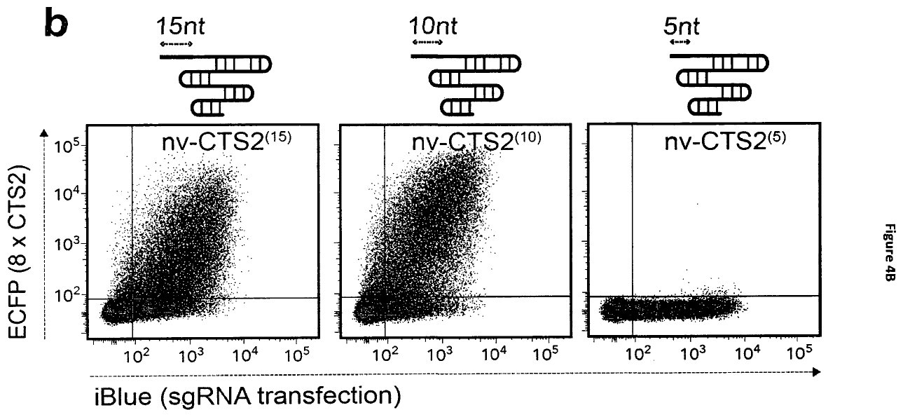

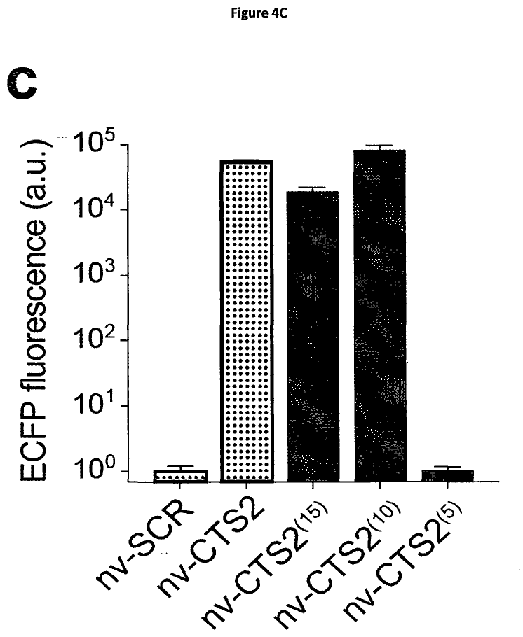

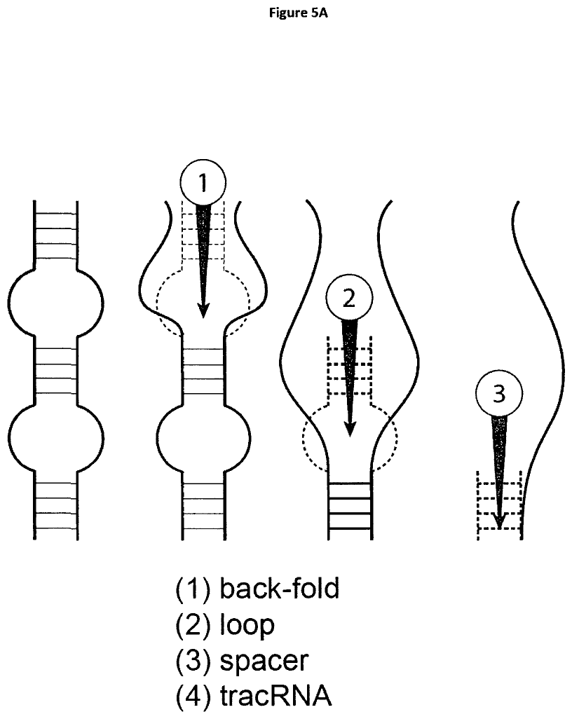

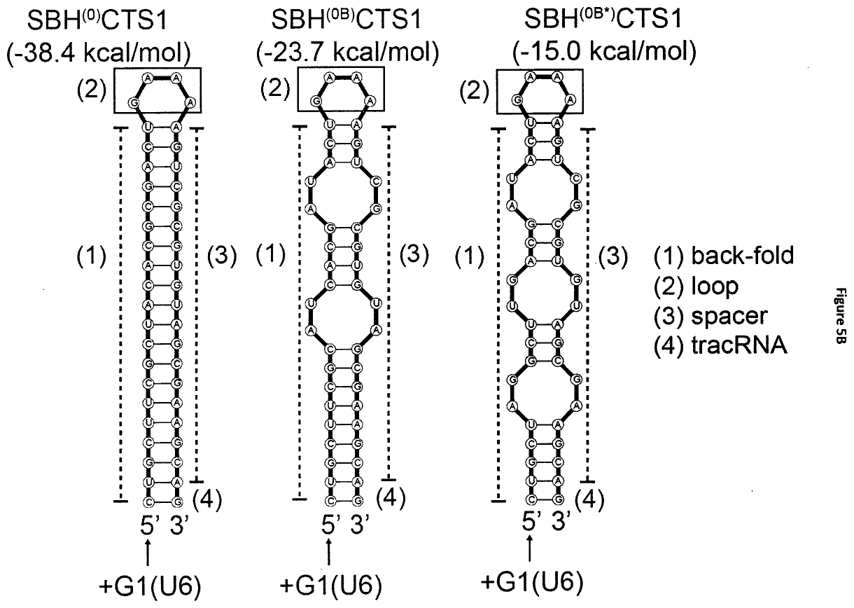

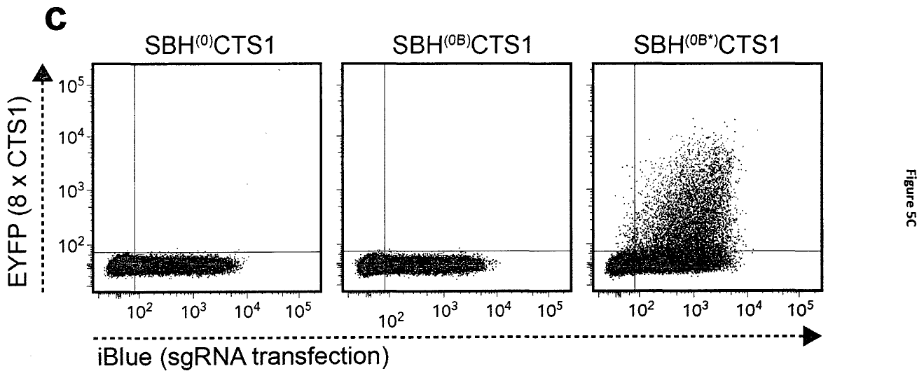

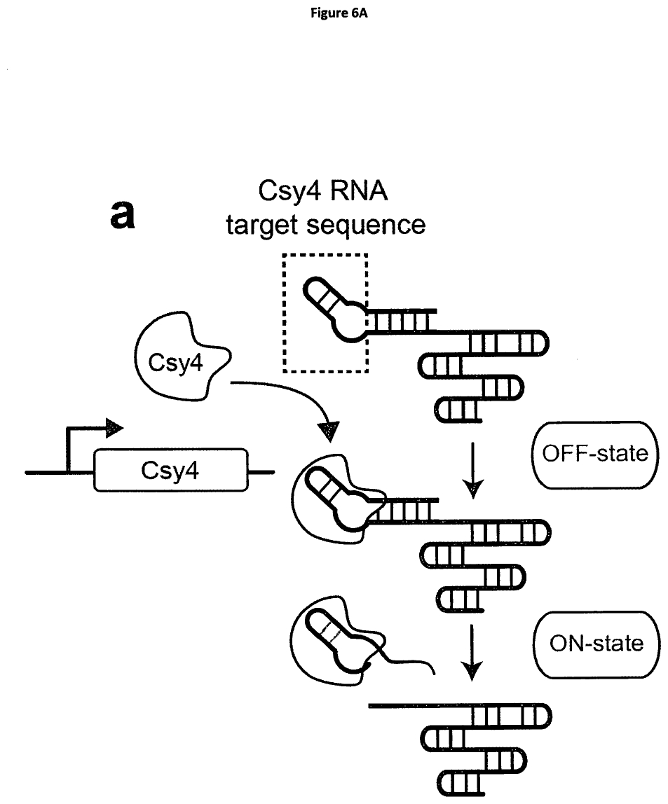

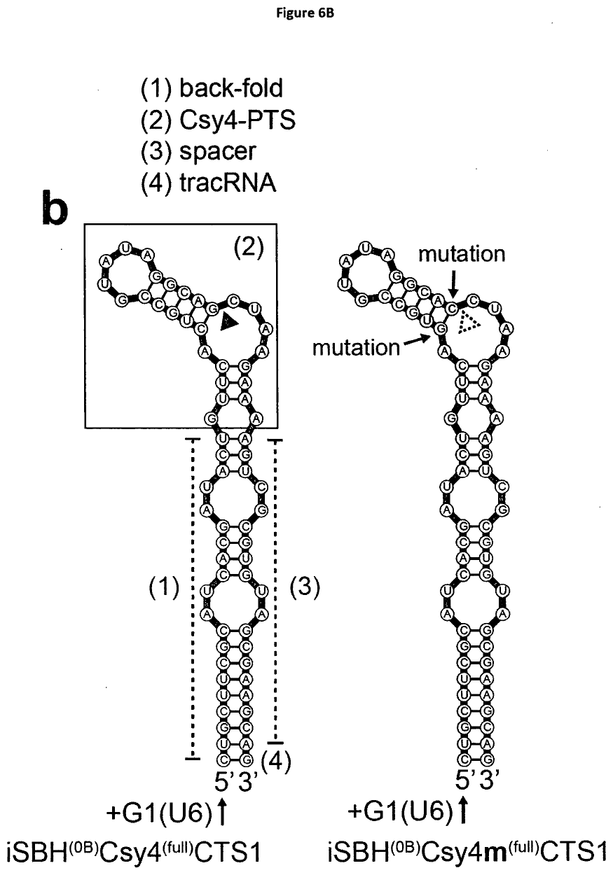

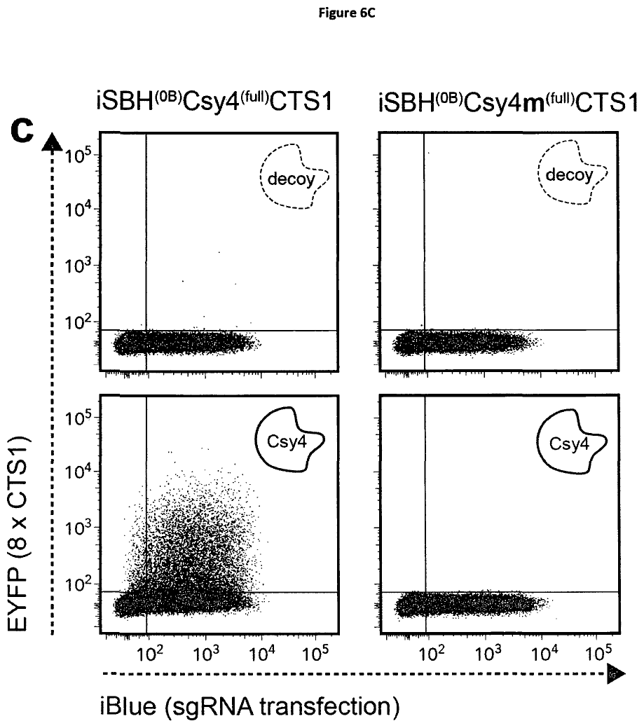

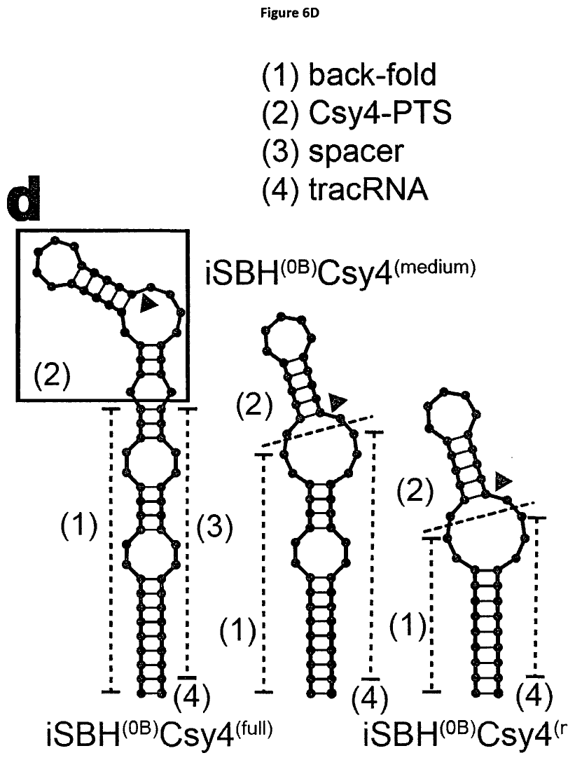

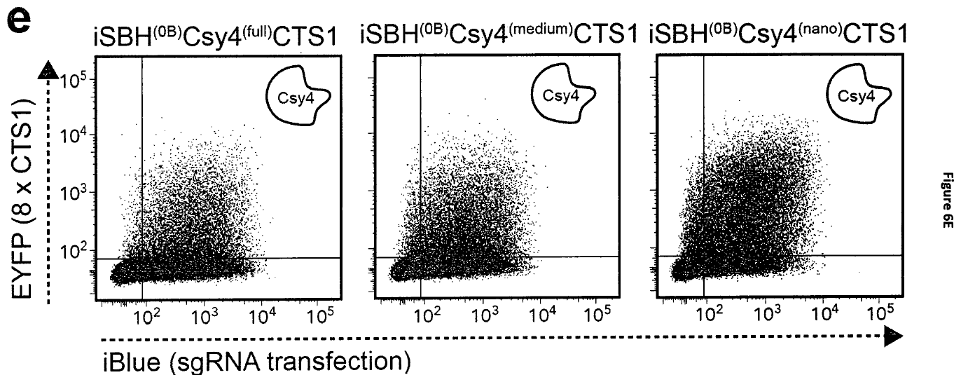

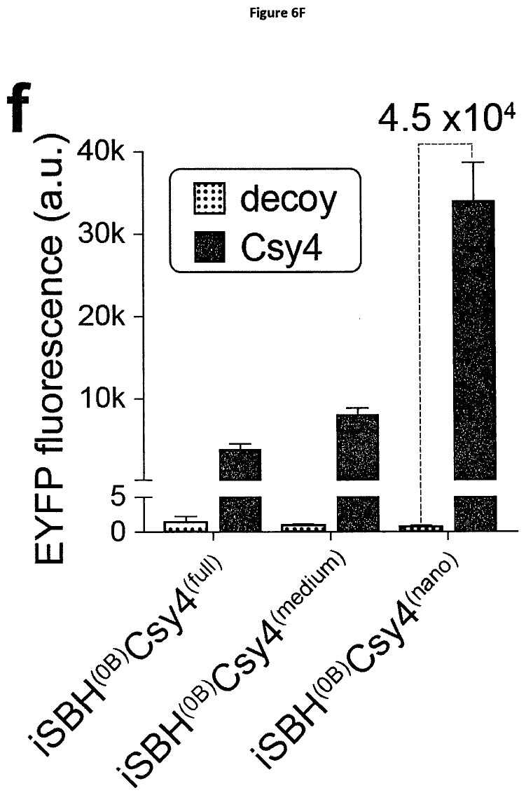

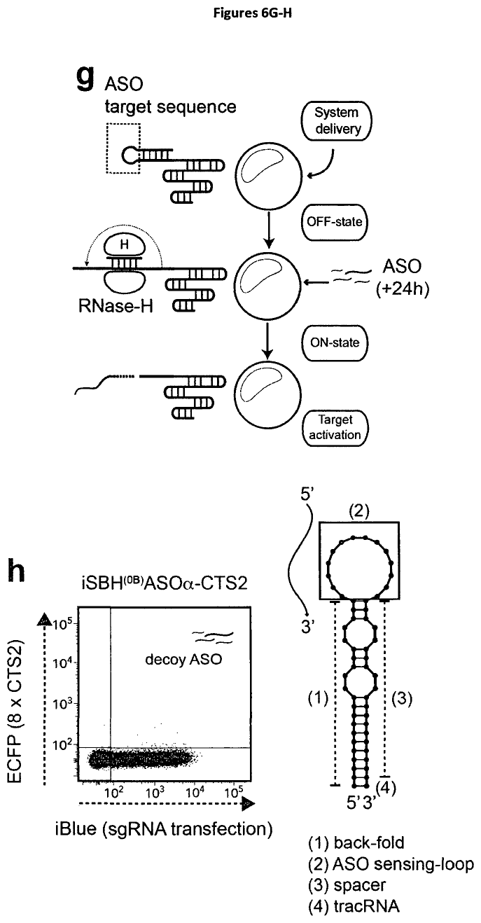

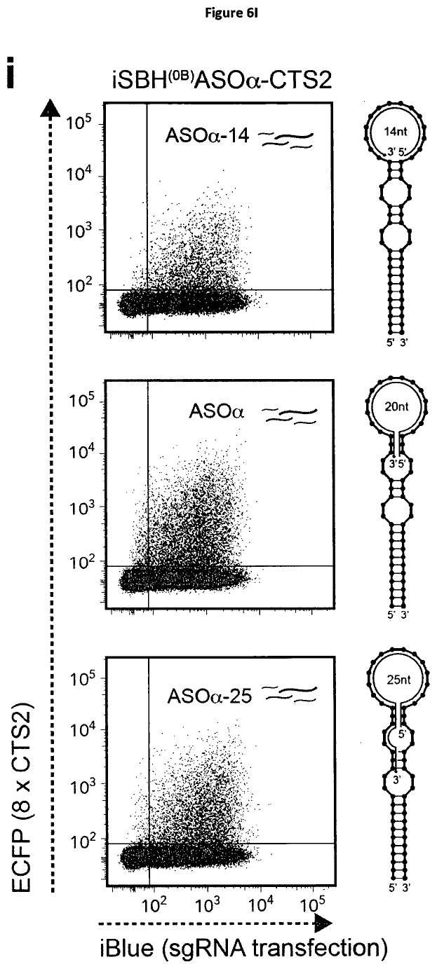

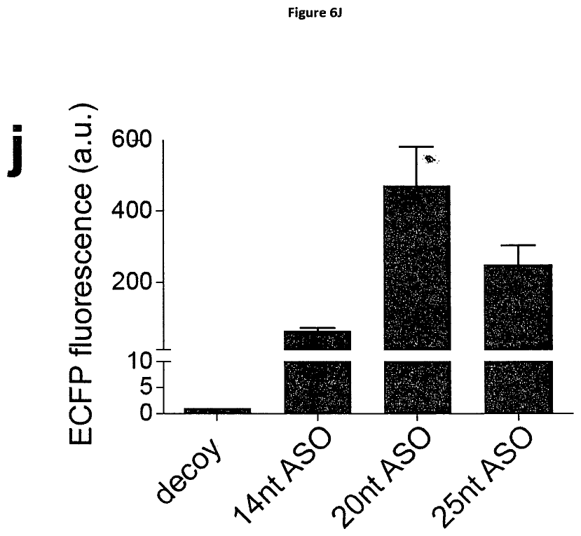

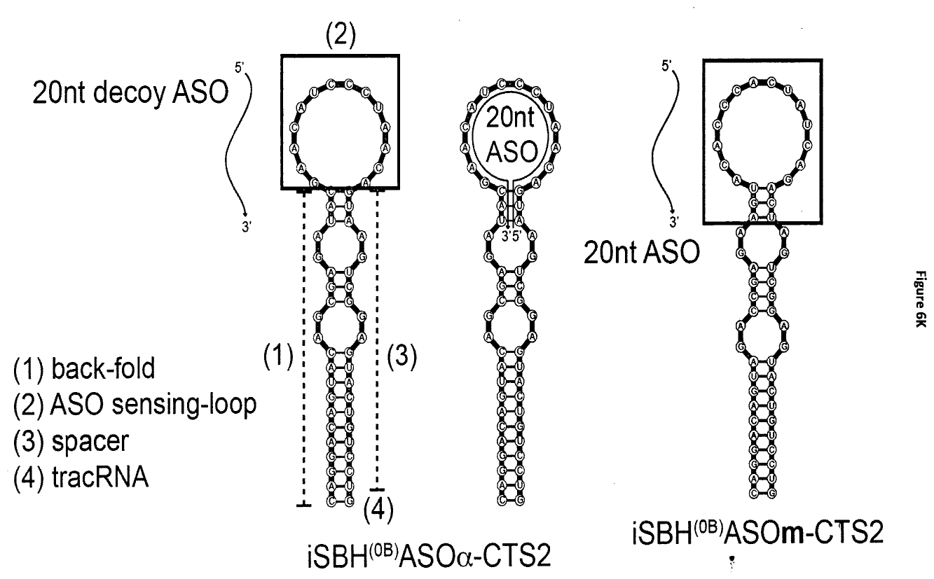

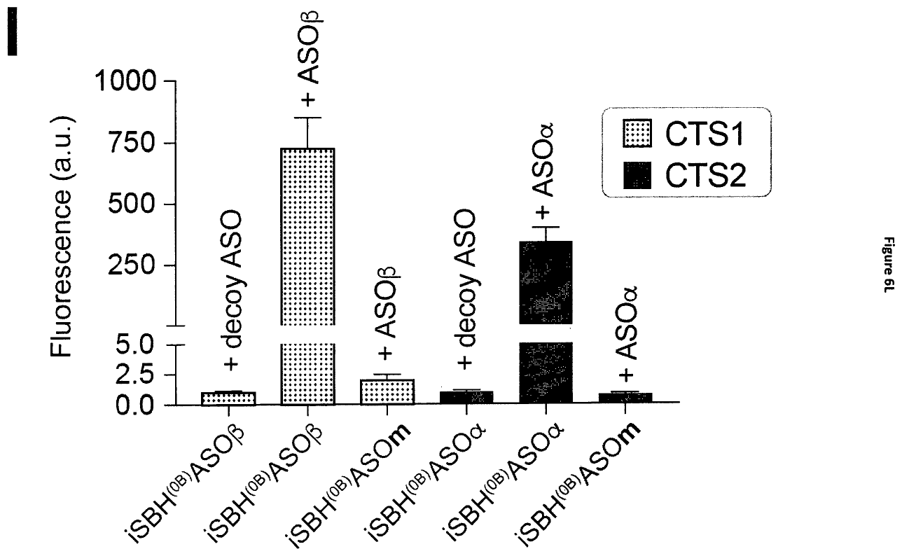

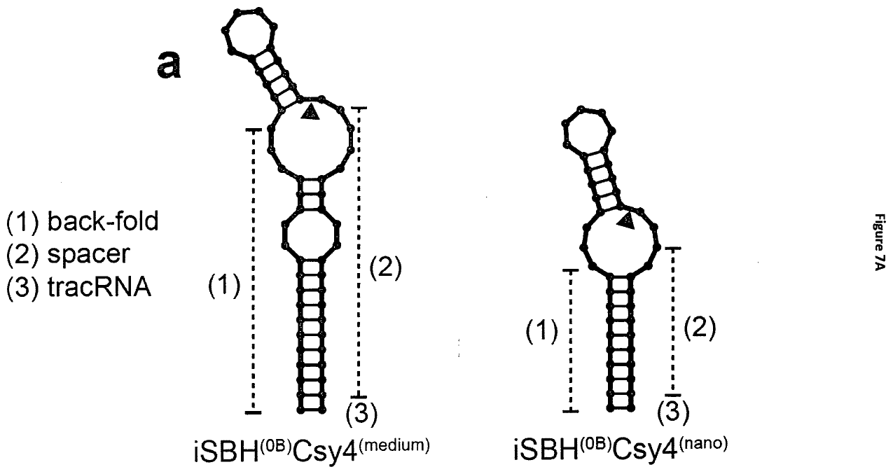

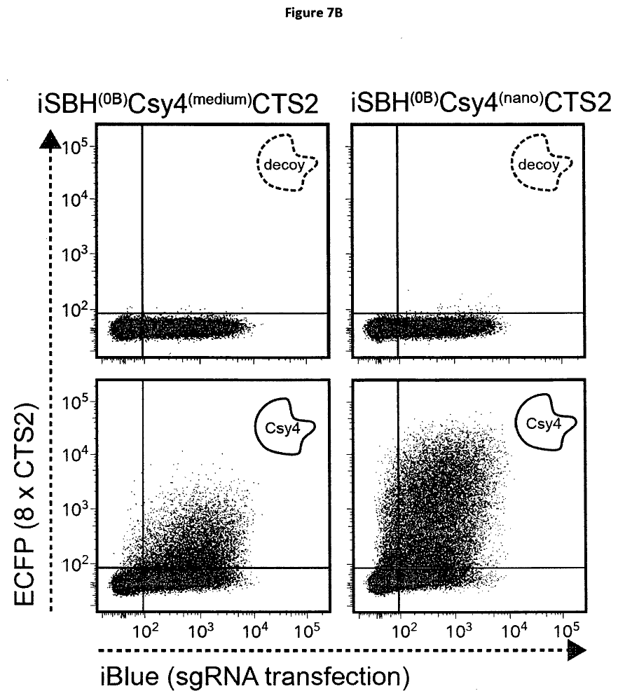

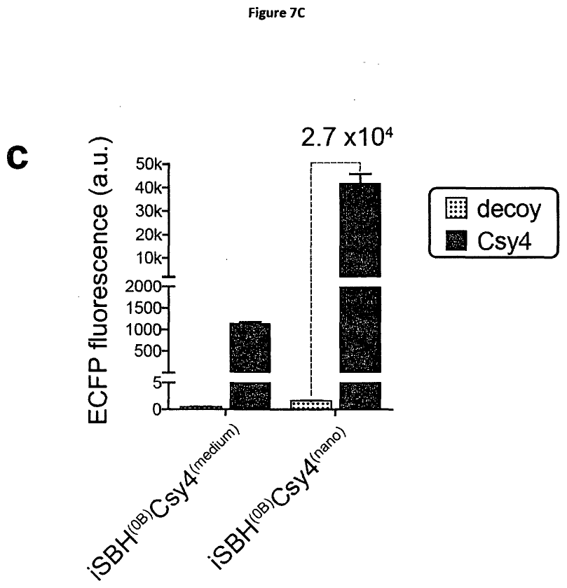

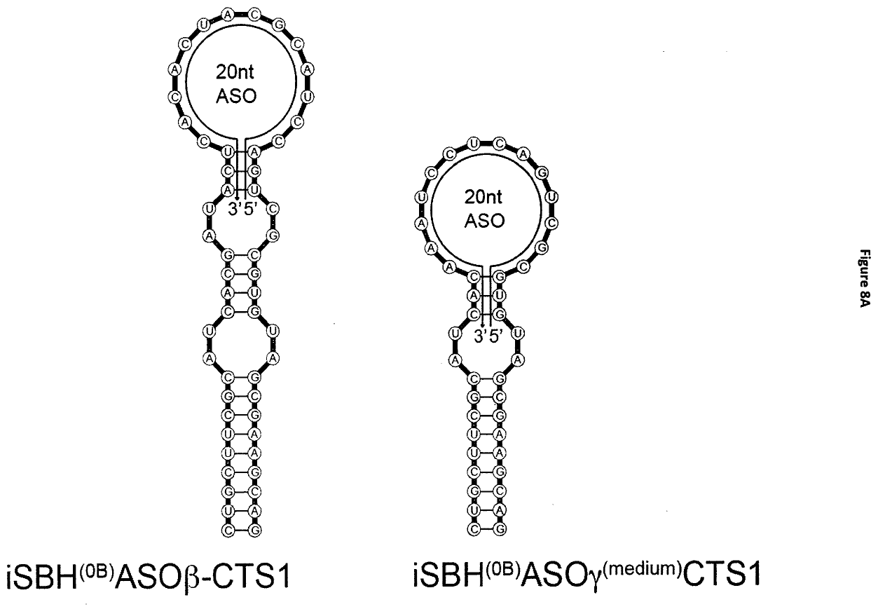

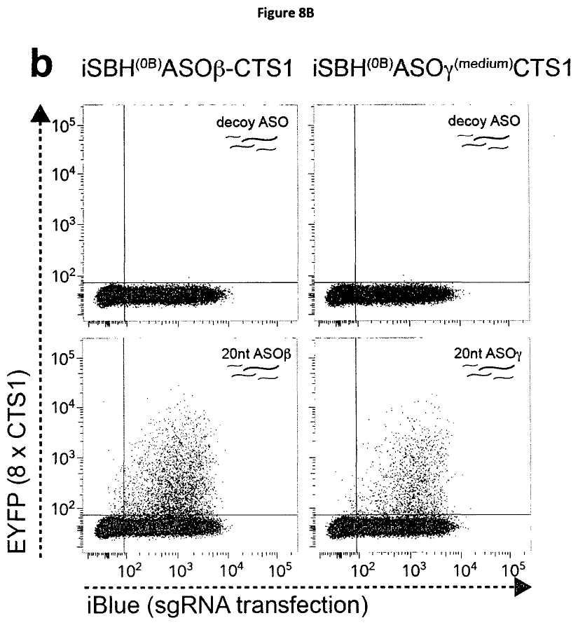

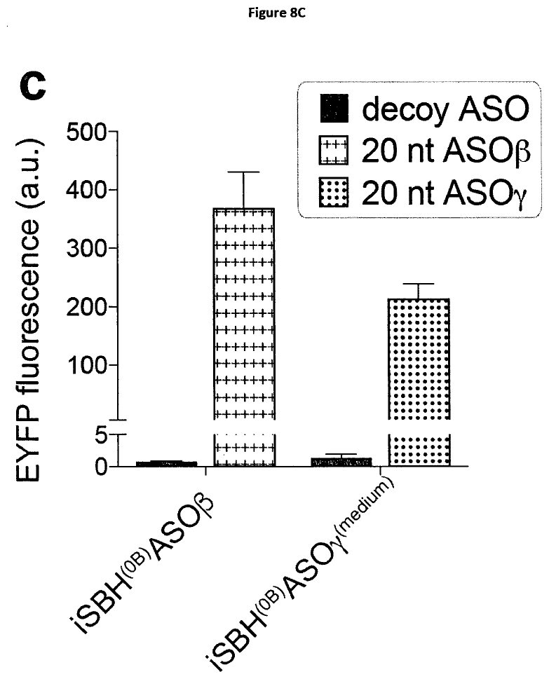

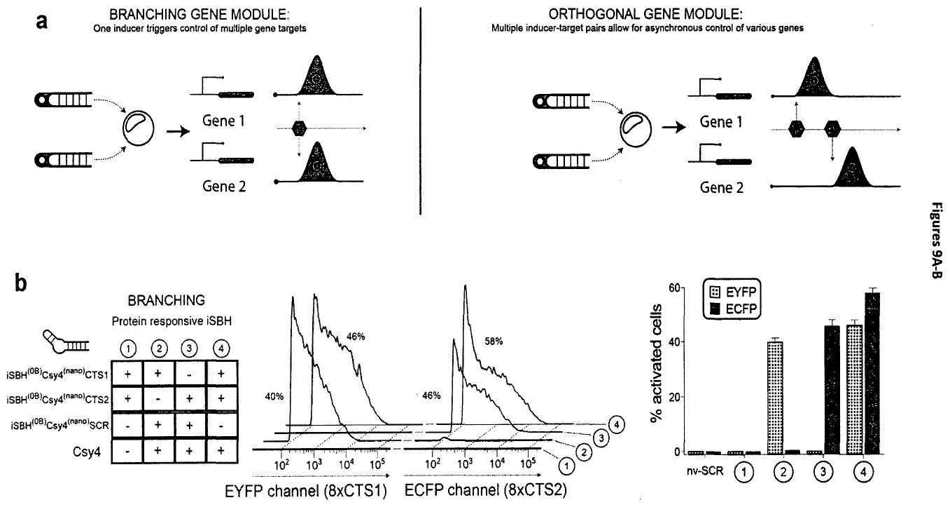

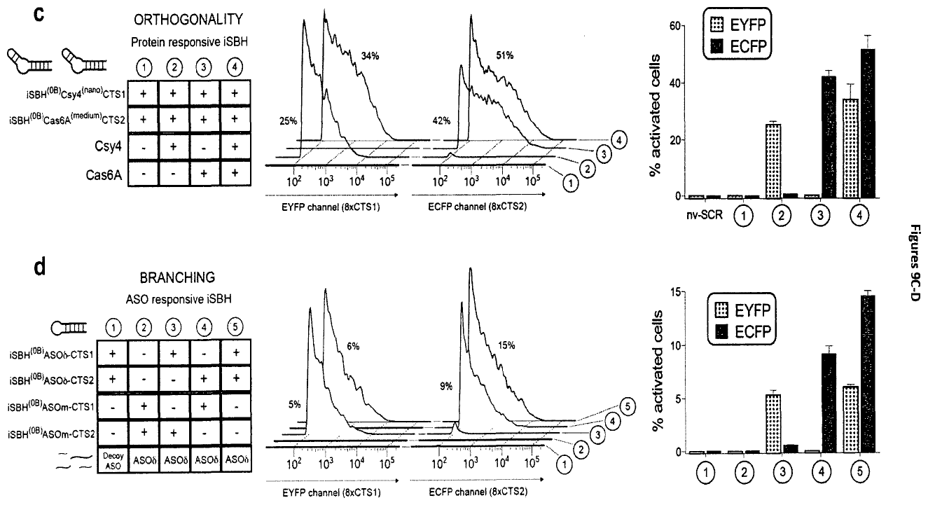

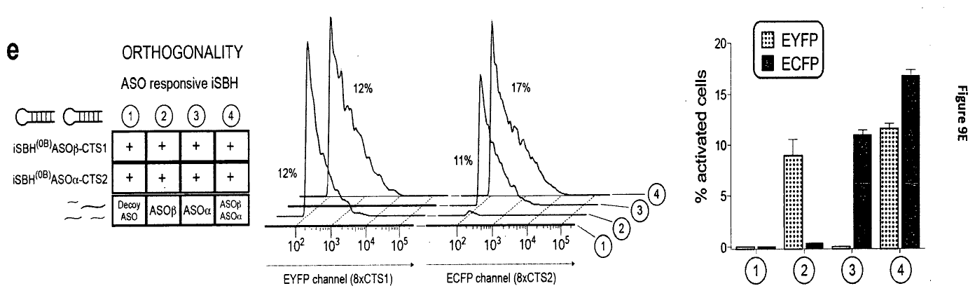

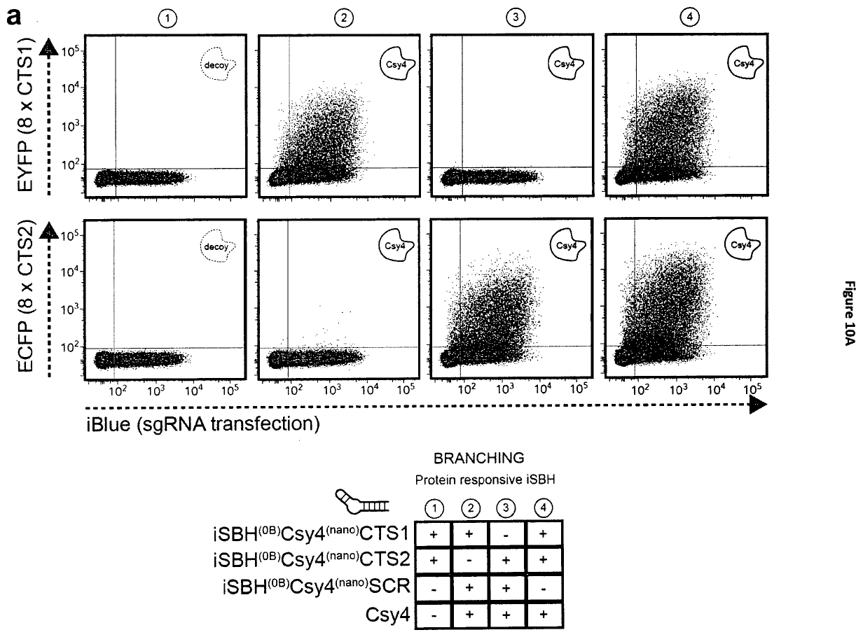

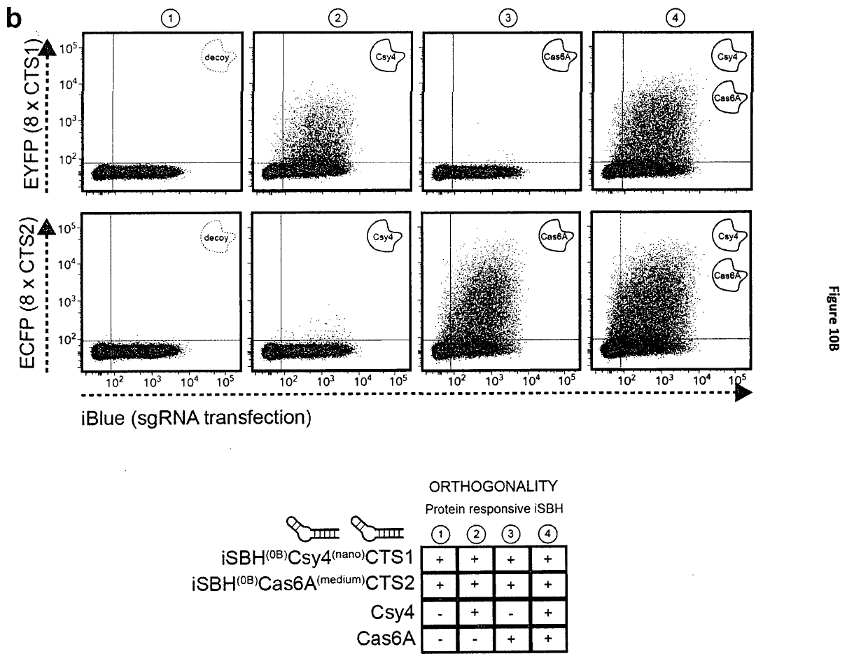

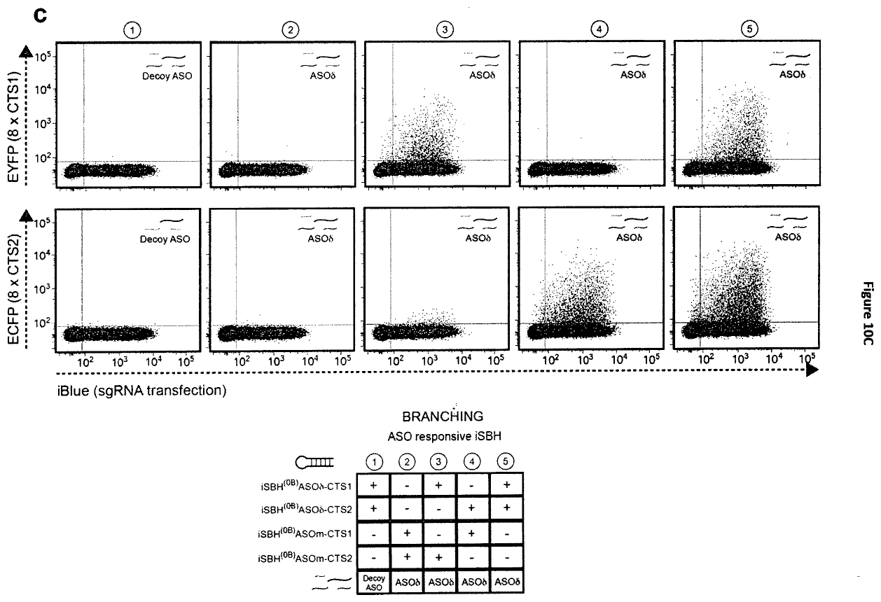

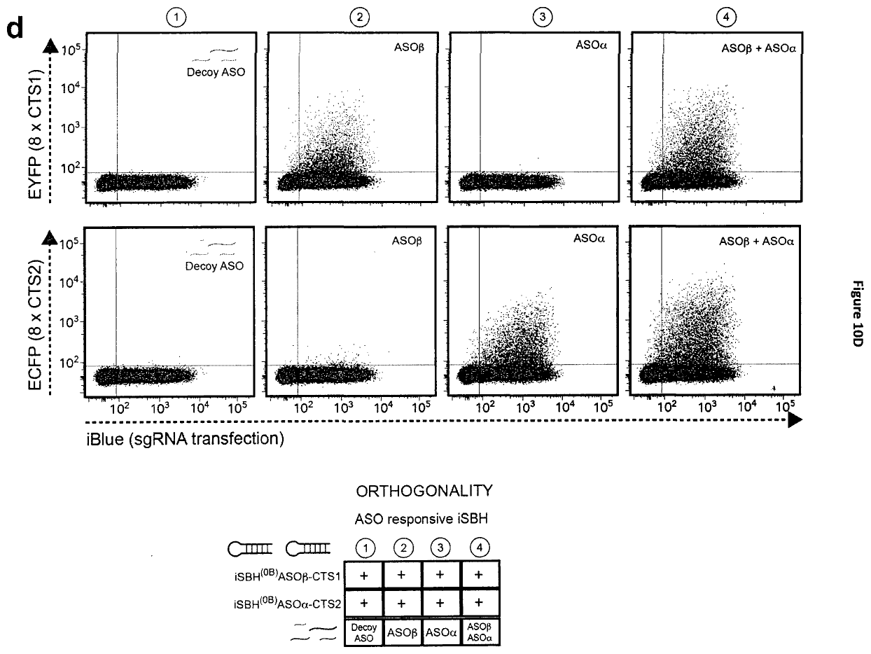

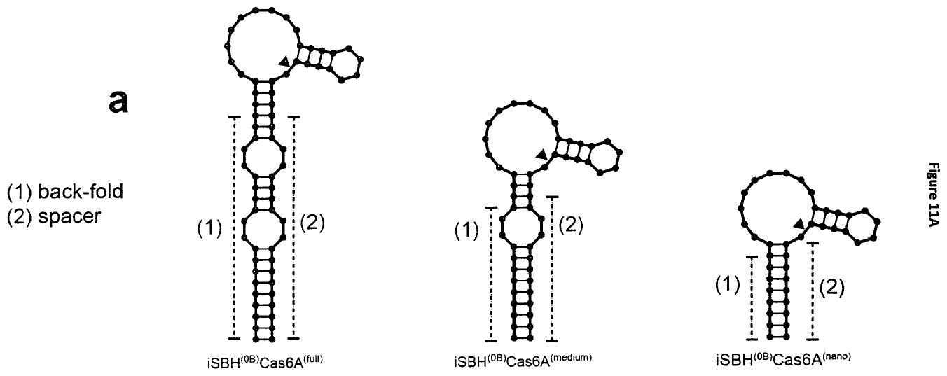

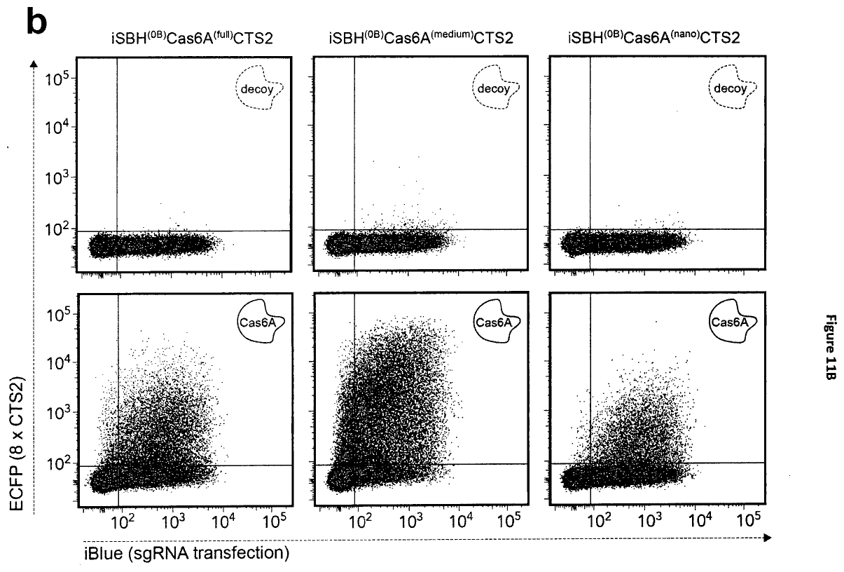

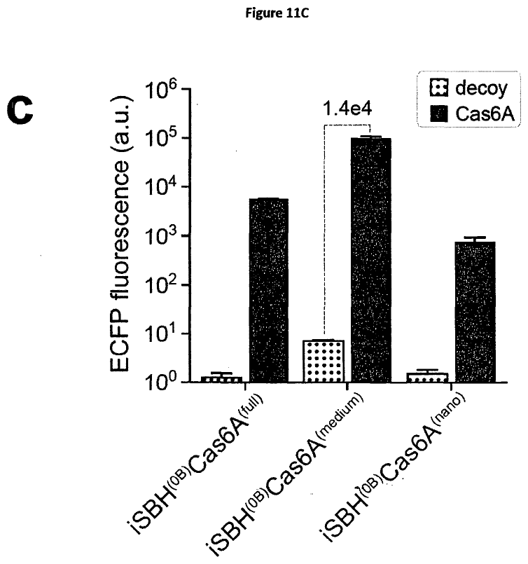

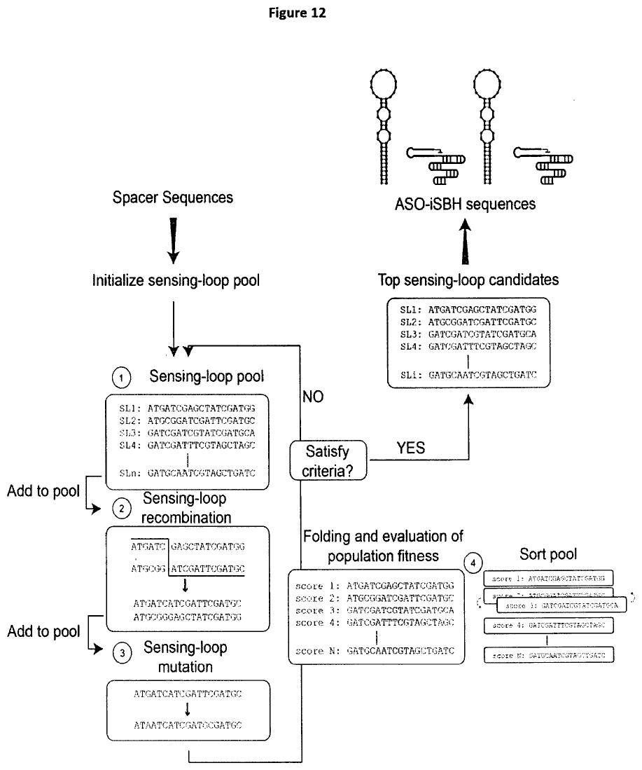

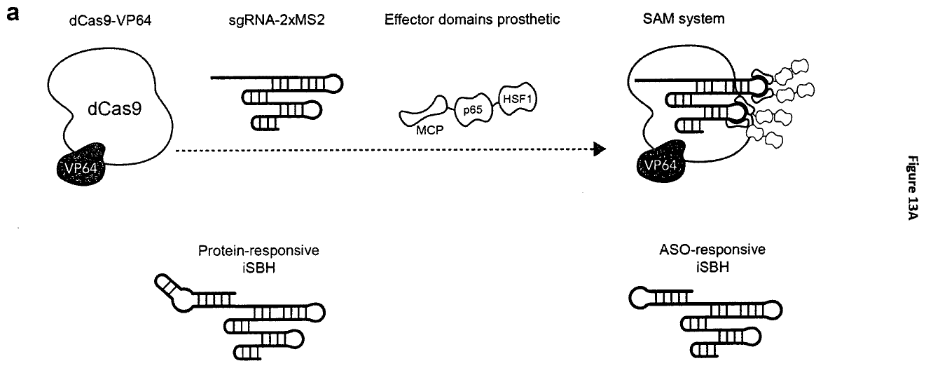

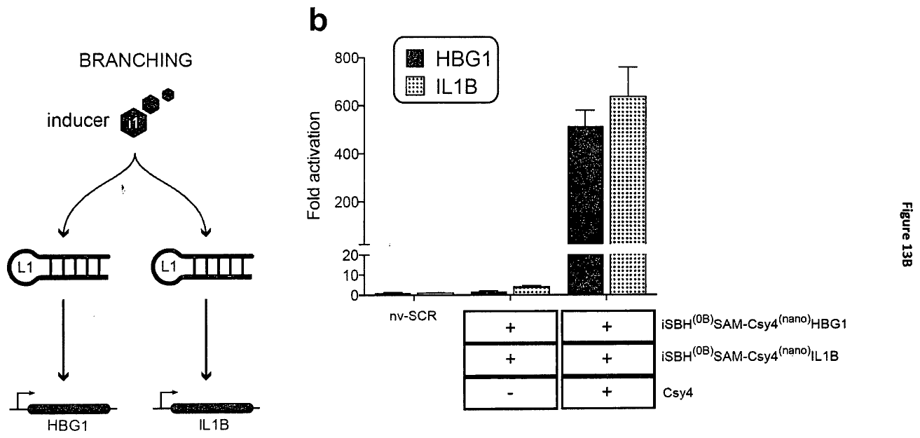

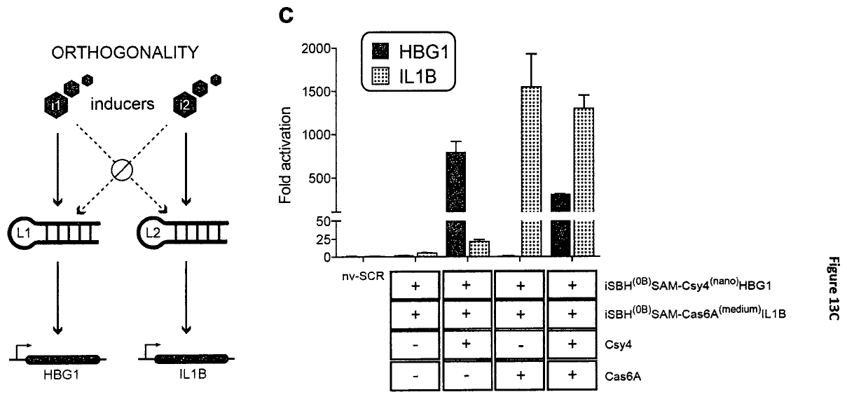

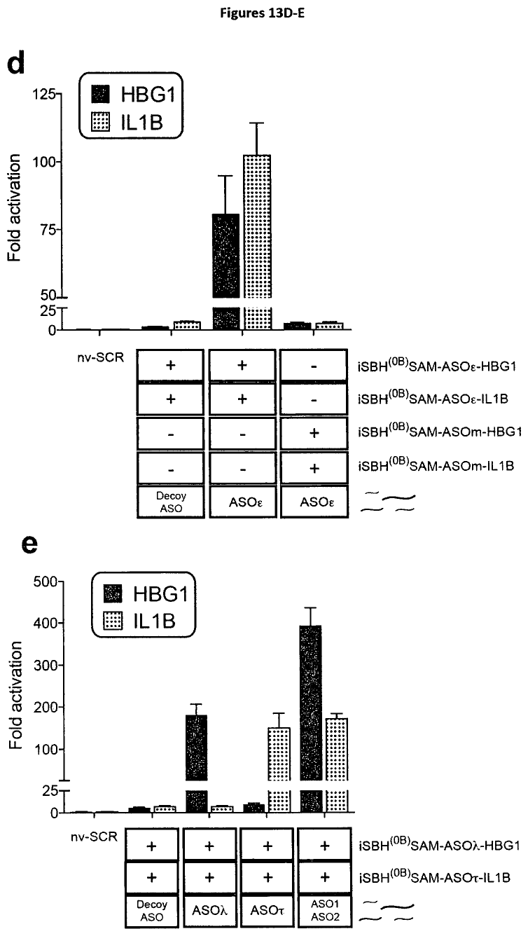

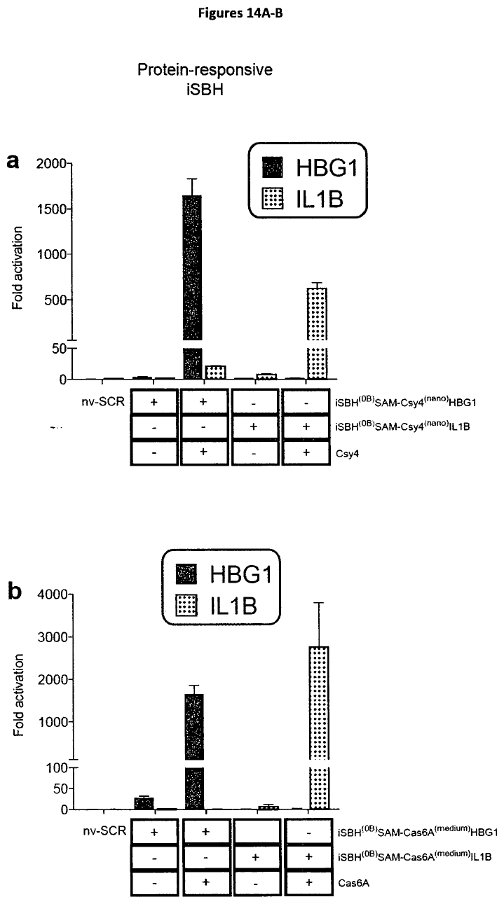

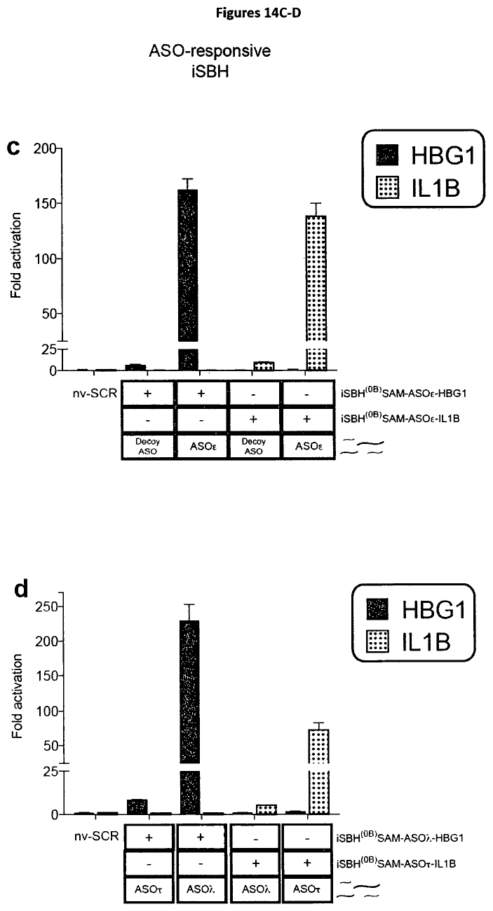

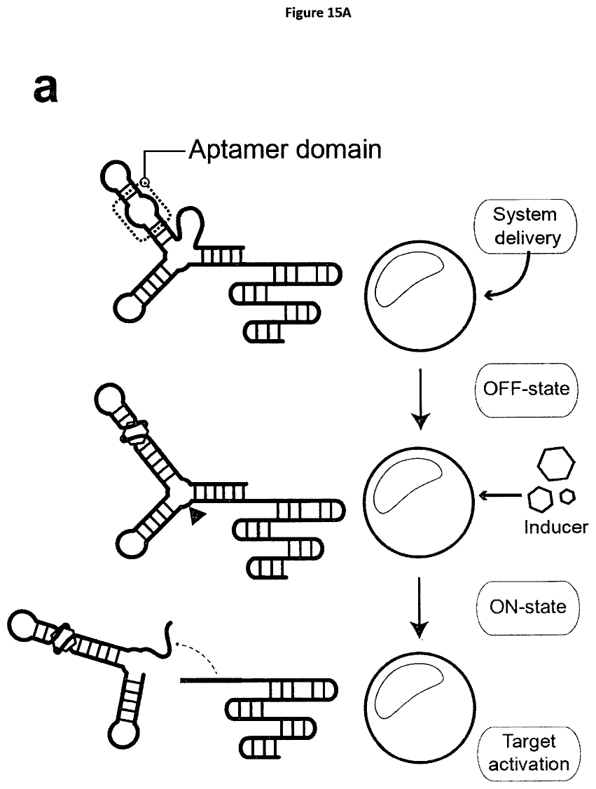

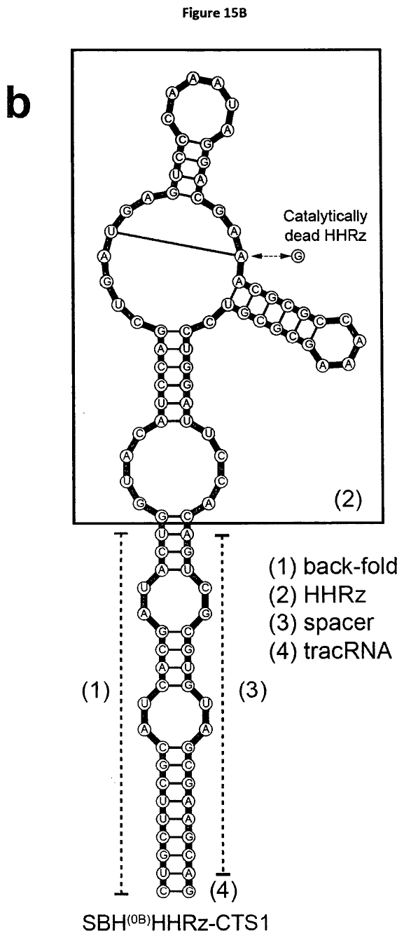

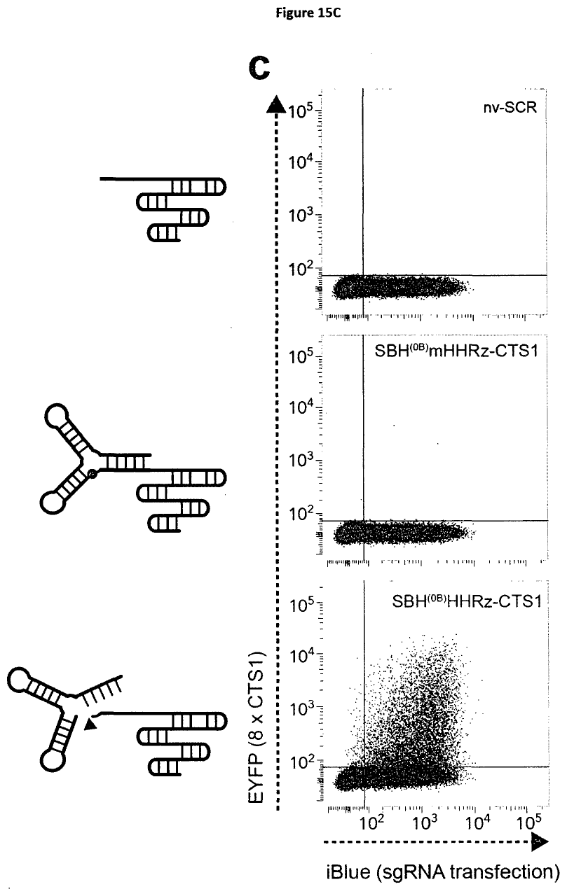

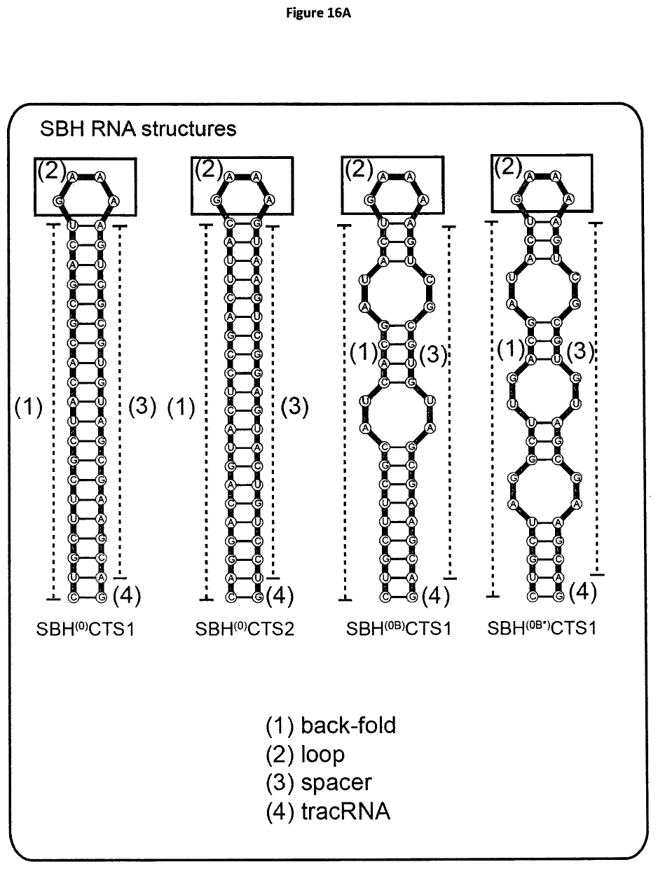

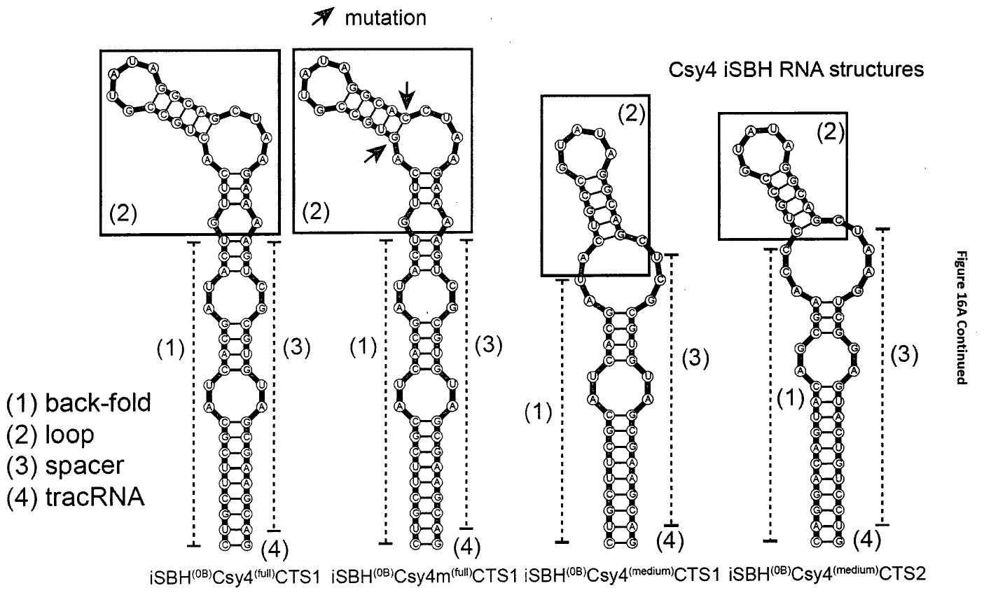

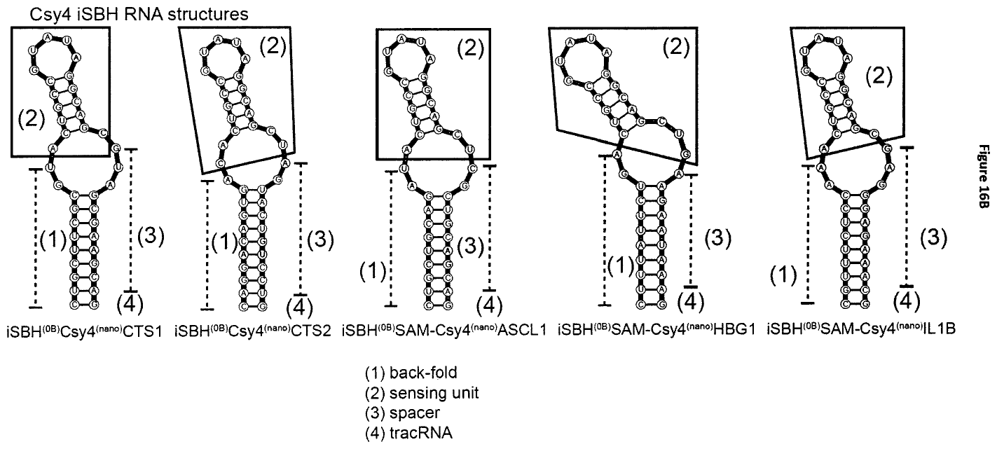

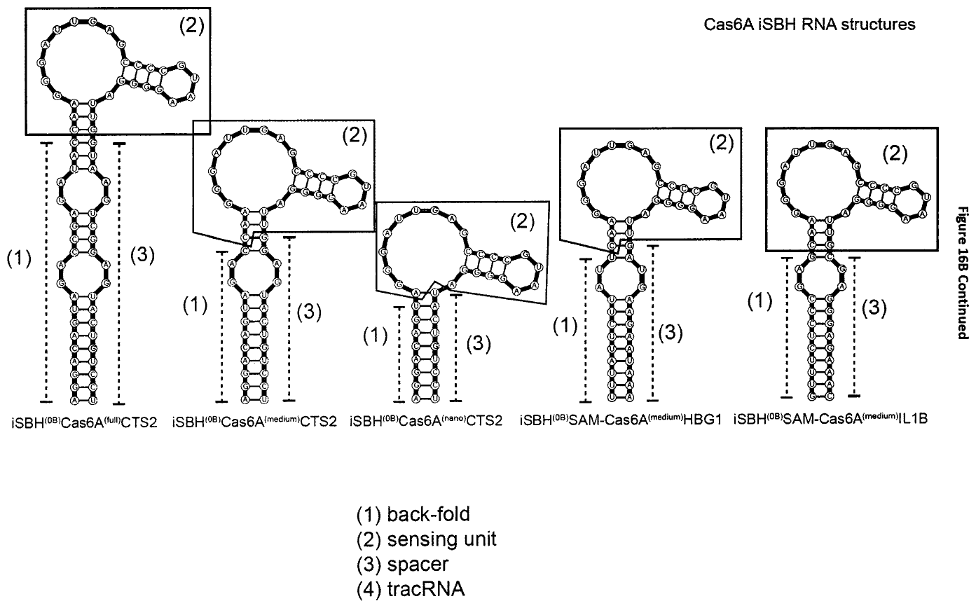

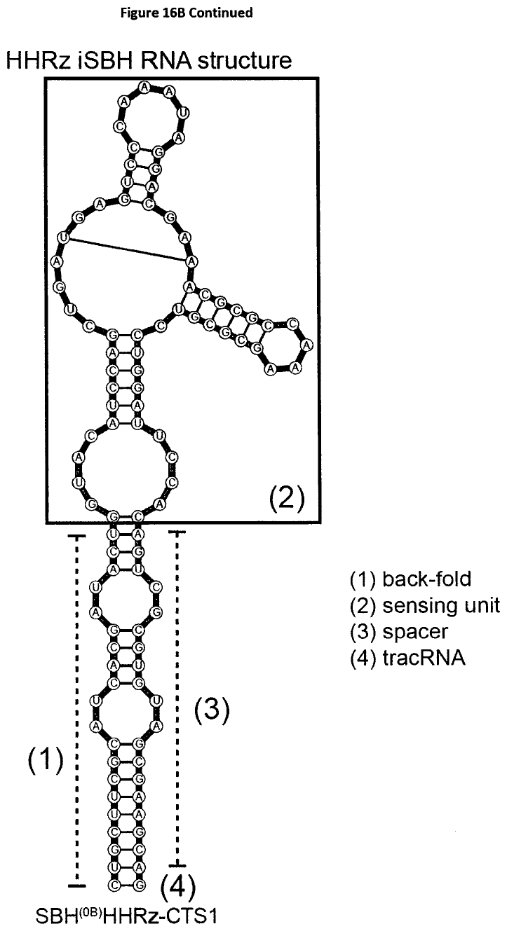

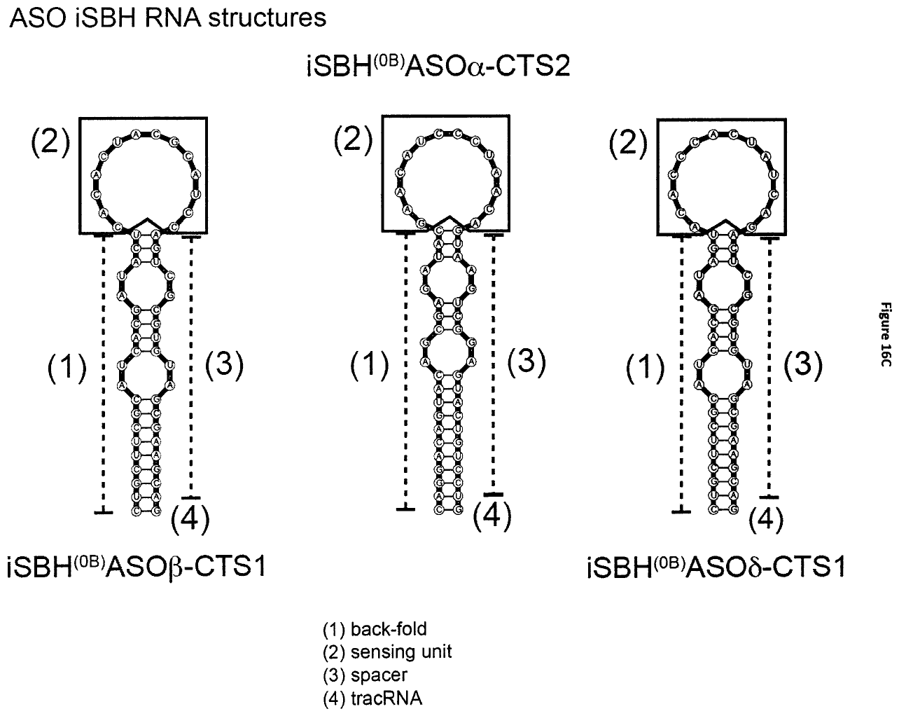

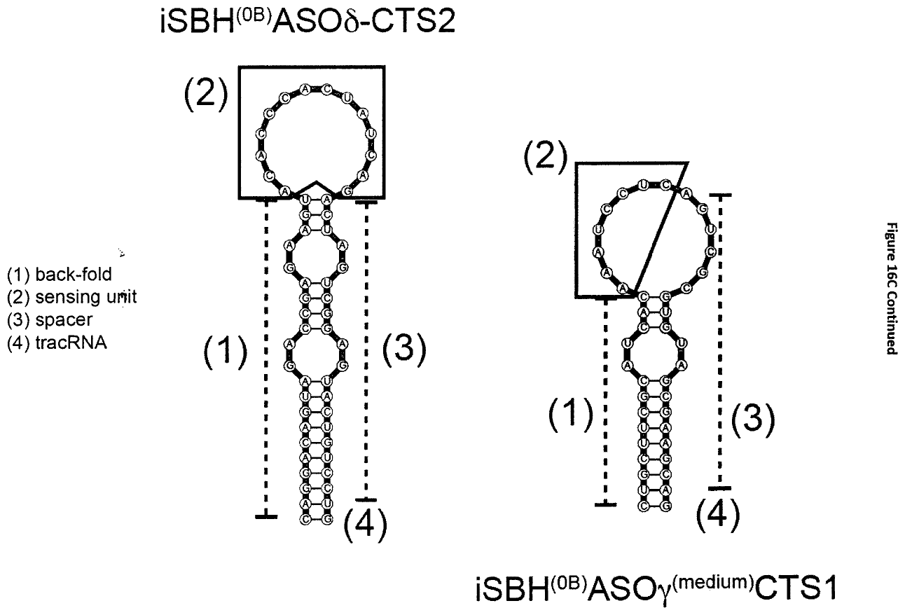

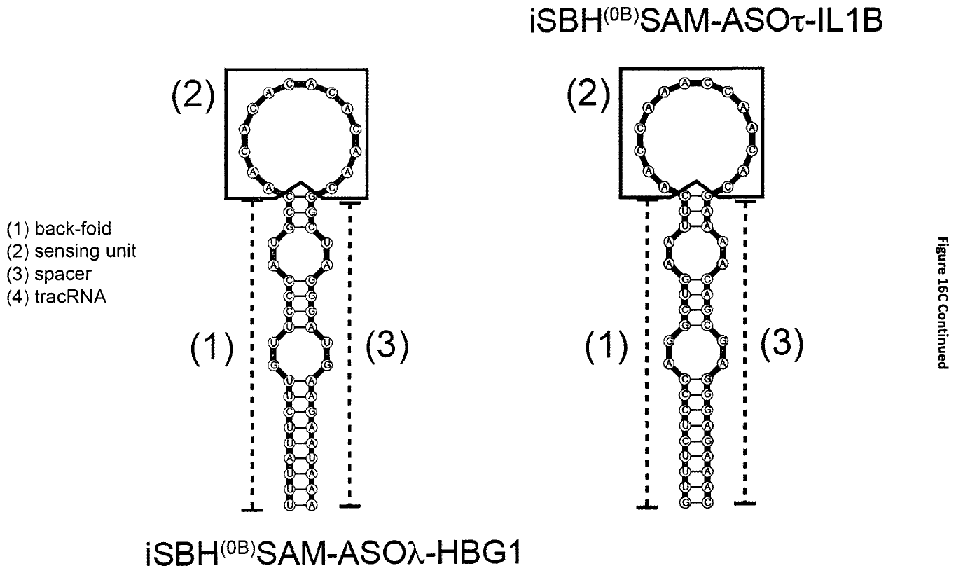

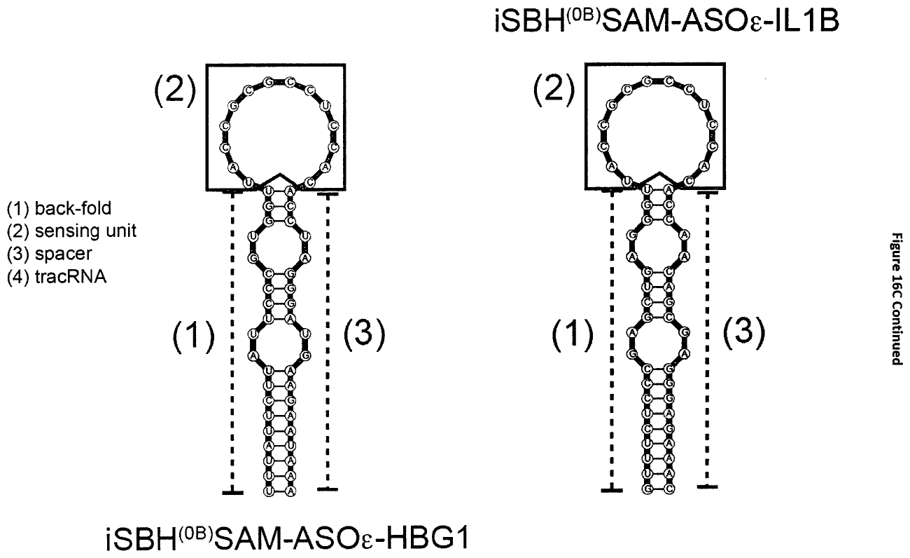

(a) HEK-293T cells were co-transfected with plasmids containing dCas9-VP64, 8×CTS1-mCMVp-EYFP or 8×CTS2-mCMVp-ECFP, and the following SBH-sgRNAs: i)) SBH⁽⁰⁾CTS1 and SBH⁽⁰⁾CTS2 (silent prototype SBH-sgRNAs with full spacer coverage); ii) SBH^{(ctrl-1)}CTS1 and SBH^{(ctrl-1)}CTS2 (control SBH-sgRNAs with accessible spacer and offset 5′ end 10 bp hairpin structure; see also FIG. 1); iii) SBH^{(ctrl-2)}CTS1 and SBH^{(ctrl-2)}CTS2 (control SBH-sgRNAs with accessible spacer and offset 5′ end 20 bp hairpin structures); SBH^{(ctrl-3)}CTS1 and SBH^{(ctrl-3)}CTS2 (control SBH-sgRNAs with a scramble back-fold segment). Representative flow cytometry scatter plots (48 h post-transfection) show reporter activation (EYFP, ECFP channels) plotted against sgRNA transfection (iBlue channel) for CTS1 and CTS2 spacers. (b) Graph represents percentage of activated (EYFP or ECFP positive) sgRNA expressing (iBlue positive) cells for all conditions in (a) and FIG. 1b (n=3, mean+/−SD).

FIG. 4: Effect of sgRNA spacer shortening on CRISPR-TA activity.

(a) Progressive shortening (5′ to 3′) of a native sgRNA spacer length (nv-CTS2=native 20 nt long spacer; nv-CTS2^{(15, 10, 5)}=truncated spacers). (b) Representative flow cytometry scatter plots showing the impact of incremental spacer shortening (nv-CTS2^{(15, 10, 5})) on reporter gene activation (axes represent ECFP reporter expression against sgRNA plasmid transfection for the three truncated sgRNA spacers). (c) Quantification of ECFP reporter fluorescence from matched experiments using the following sgRNAs: nv-SCR (control 20 nt scramble spacer), nv-CTS2 (matching 20 nt spacer), nv-CTS2⁽¹⁵)(matching 15 nt spacer), nv-CTS2⁽¹⁰⁾ (matching 10 nt spacer), nv-CTS2⁽⁵⁾(matching 5 nt spacer) (n=3, mean+/−SD, a.u. arbitrary units).

FIG. 5: Optimization of SBH thermodynamic stability.

(a) Hypothetical framework showing the expected low energy stepwise strand separation as a consequence of bulge incorporation into the SBH stem architecture. (b) Sequence and RNA secondary structure corresponding to the prototype SBH⁽⁰⁾CTS1 design, and alternative architectures containing two (SBH^{(0B)}CTS1) and three (SBH^{(0B*)}CTS1) stem bulges. The calculated minimal free energy of each structure is shown in brackets. (c) Representative flow cytometry scatter plots (EYFP reporter fluorescence against iBlue sgRNA transfection) reveals complete CRISPR-TA silencing in both SBH⁽⁰⁾CTS1 and SBH^{(0B)}CTS1 designs. In contrast, addition of a third bulge renders the sgRNA active, presumably due to excessive destabilisation of the stem structure.

FIG. 6: Design and implementation of inducible iSBH-sgRNAs.

(a) Conceptual framework underlying conditional spacer release using genetically encoded inducers (endoribonucleases). Grafting the Csy4 RNA motif onto the SBH stem allows OFF- to ON-state transition in the presence of the CRISPR-associated endoribonuclease Csy4. (b) RNA secondary structure and sequence identity of the Csy4-responsive iSBH^{(0B)}Csy4^{(full)}CTS1 and the control mutant variant iSBH^{(0B)}Csy4m^{(full)}CTS1 (point mutation (yellow) renders the recognition sequence insensitive to Csy4 cleavage). Red arrow indicates Csy4 cleavage site. (c) Representative flow cytometry scatter plots (EYFP reporter fluorescence against iBlue sgRNA transfection) reveal complete silencing in the absence of inducer (decoy=empty plasmid) or when using the mutant iSBH. Robust reporter expression is observed in the presence of Csy4. (d-f) Optimisation of Csy4-iSBH designs. RNA secondary structures (CTS1 spacer; red arrow Csy4 cleavage site) (d) and representative CRISPR-TA assay flow cytometry scatter plots (+Csy4 ON-state) (e) for iSBH^{(0B)}Csy4 full, medium, and nano stems. Quantification of EYFP reporter expression using the three iSBH variants in the presence of a decoy plasmid or Csy4 inducer from three biological replicates (n=3, mean+/−SD, a.u. arbitrary units) (f). (g) Implementation of ASO-responsive iSBH designs for temporal control of CRISPR-TA activity. This platform enables delayed sgRNA activation by means of externally delivered cognate ASOs. (h) CRISPR-TA assay (ECFP reporter expression flow cytometry) in the presence of iSBH^{(0B)} ASOα-CTS2 and a decoy ASO with scrambled sequence (purple, incompatible base pairing with the sensing loop). (i-j) Impact of ASO length on the activation of iSBH^{(0B)} ASOα-CTS2 sgRNA (ON-state). Representative flow cytometry scatter plots and corresponding ASO:iSBH pairing diagrams using 14, 20 and 25 nucleotide long ASO inducers (red) (i). Quantification of ON-state activation (ECFP fluorescence) for the conditions shown in (h) and (i) (n=3, mean+/−SD, a.u. arbitrary unit) (j). (k, l) Analysis of ON-state activation and OFF-state inhibition using the optimal 20 nt ASO inducer design on two target genes (CTS1-EYFP and CTS2-ECFP). For each CTS three experimental conditions were compared: decoy ASO/iSBHsgRNA; matching ASO/iSBH-sgRNA; matching ASO/mutant iSBH-sgRNA (non-matching sensing loop) (k). Quantification of reporter activation from three biological replicates for each condition. No activation above background was detected in control conditions (decoy ASO or matching ASO+iSBH^{(0B)} ASOm-CTS), while robust CRISPR-TA-mediated reporter expression was elicited by the presence of active ASOs (l).

FIG. 7: Optimisation of Csy4-responsive iSBH designs for CTS2 spacer.

(a) RNA secondary structures of SBH^{(0B)}Csy4 medium, and nano stems (CTS2 spacer; red arrow=Csy4 cleavage site). (b) Representative CRISPR-TA assay flow cytometry scatter plots for iSBH^{(0B)}Csy4^{(medium)}CTS2 and iSBH^{(0B)}Csy4^{(nano)}CTS2 sgRNAs in the absence (OFF-state, top) and presence (ON-state, bottom) of Csy4. (c) Quantification of ECFP reporter expression using the two iSBH variants in the presence of a decoy plasmid or Csy4 inducer from three biological replicates (n=3, mean+/−SD, a.u. arbitrary units).

FIG. 8: Evaluation of two ASO responsive iSBH designs.

(a) Sequence and RNA secondary structure of ASO-iSBH constructs with different stem length architectures. The iSBH^{(0B)} ASOβ-CTS1 design was generated by replacing the 4 nt loop in SBH^{(0B)}CTS1 with a 14 nt sensing loop complementary to ASOβ. Extrapolating from the observation that shorter protein-responsive iSBH stems tend to display superior behaviour, we also engineered iSBH^{(0B)} ASOγ^{(medium)}-CTS1 by converting the distal bulge into a 14 nt ASO sensing-loop, which incorporates 6 spacer nucleotides and was evolved to enforce an open ssRNA secondary structure. (b) Representative flow cytometry scatter plots (ECFP reporter fluorescence against iBlue sgRNA transfection) using the two ASO-responsive iSBH sgRNAs designs, in the presence of a decoy ASO (OFF-state) or active cognate ASO (ON-state). (c) Quantification of reporter fluorescence for all conditions shown in (b) (n=3 biological replicates, mean+/−SD, a.u. arbitrary units).

FIG. 9: iSBH-based assembly of branching and orthogonal gene network modules

(a) Schematic representation of branching and orthogonal gene network modules using iSBHsgRNAs. iSBH-sgRNAs programmed with specific sensing loops (purple/orange) and/or spacer identities (blue/green) enable rapid generation of parallel and orthogonal inducer:target gene pairs, facilitating synchronous or asynchronous control of transcriptional programs. (b) Concurrent activation of two target genes using protein-responsive iSBH sgRNAs (branching module). Csy4-responsive iSBH^{(0B)}Csy4^{(nano)}CTS1 and iSBH^{(0B)}Csy4^{(nano)}CTS2 were co-transfected with dCas9-VP64 and a dual-reporter system (CTS1-EYFP/CTS2-ECFP) in the absence [1] or presence [4] of the inducer. To confirm Csy4-mediated specific activation of CTS1 and CTS2 target genes, each corresponding iSBH-sgRNA was co-transfected with a control iSBH-sgRNA carrying a scramble spacer [2 and 3]. (c) Orthogonal activation of two target genes using protein-responsive iSBH sgRNAs (orthogonal module). Csy4- and Cas6A-responsive) iSBH^{(0B)}Csy4^{(nano)}CTS1 and iSBH^{(0B)} Cas6A^{(medium)}CTS2 respectively, were co-transfected in the absence of any inducer [1], the presence of each individual inducer [2 and 3], or a combination of the two [4]. Specific activation of each inducer:target gene pair was observed with no interference between orthogonal branches. (d) Parallel activation of target genes using ASO-responsive iSBH sgRNAs (branching module). iSBH^{(0B)} ASOδ-CTS1 and iSBH^{(0B)} ASOδ-CTS1 containing a shared sensing loop were co-transfected with dCas9-VP64 and the dual-reporter system. Decoy ASO [1] or trigger ASO [5] were delivered to cells 24 h post-transfection. Parallel experiments using iSBH-sgRNAs with mutant sensing loops (iSBH^{(0B)} ASOm-CTS1 and iSBH^{(0B)} ASOm-CTS2) were carried out to confirm the specificity of the observed effects [2, 3 and 4]. (e) Implementation of an orthogonal gene activation module using ASO-responsive iSBH-sgRNAs. iSBH^{(0B)} ASOβ-CTS1 and iSBH^{(0B)} ASOα-CTS2 containing distinct sensing loop units (orthogonal sequences) were supplemented 24 h post-transfection with a decoy ASO [1], ASOβ [2], ASOα [3] or a combination of ASOβ+ASOα [4]. Flow cytometry analysis revealed specific reporter output activation for each inducer:target gene pair, without any apparent crosstalk between branches. For all experiments, histograms count double positive events in each reporter channel (iBlue/EYFP or iBlue/ECFP). The graphs show percentage of activated cells (EYFP and/or ECFP positive) among the entire sgRNA transfected population (iBlue positive) from three biological replicates for each condition; mean+/−SD; see also FIG. 10a-d for representative raw data flow cytometry scatter plots. nv-SCR refers to a control native sgRNA with a scramble spacer sequence.

FIG. 10: iSBH based implementation of branching and orthogonal gene network modules

Raw flow cytometry data related to FIG. 9. (a) Branching gene module with protein-iSBH. (b) Orthogonal gene module with protein-iSBH. (c) Branching gene module with ASO-iSBH. (d) Orthogonal gene module with ASO-iSBH.

FIG. 11: Optimisation of Cas6A-responsive iSBH designs for CTS2 spacer.

(a) RNA secondary structures of SBH^{(0B)} Cas6A^{(full)} sgRNA compared to the medium, and nano stems (CTS2 spacer; red arrow=Cas6A cleavage site) obtained by grafting the Cas6A RNA box onto the distal or proximal SBH^{(0B)}CTS bulge. (b) Representative CRISPR-TA assay flow cytometry scatter plots for iSBH^{(0B)} Cas6A^{(full)}CTS2, iSBH^{(0B)} Cas6A^{(medium)}CTS2 and iSBH^{(0B)} Cas6A^{(nano)}CTS2 sgRNAs in the absence (OFF-state, top) and presence (ON-state, bottom) of Cas6A. (c) Quantification of ECFP reporter expression using the three iSBH variants in the presence of a decoy plasmid or Cas6A inducer from three biological replicates (n=3, mean+/−SD, a.u. arbitrary units).

FIG. 12: In silico evolution of a common ASO sensing-loop satisfying ASO-iSBH folding conditions across multiple spacers using iSBHfold.

For an input set of spacers (sp1, sp2, etc.) the algorithm aims to evolve a shared sensing-loop which can be grafted on all SBH^{(0B)} spX while ensuring accessibility to ASO binding by enforcing structural constraints (open ssRNA loop conformation). Loops satisfying these conditions are evolved from an initial pool of 20 nt RNA sequences [1]. A custom-made genetic algorithm is then run iteratively on this pool over several generations to optimise the sensing-loop sequences. For each iteration the following steps are performed: i) initial sequences are recombined to generate offspring sequences which are added to the original parent population [2]; ii) the pool is further enriched with sequences obtained by randomly mutating the existing segments as well as new fully randomised ones [3]. iii) finally, each sequence is folded within the corresponding iSBH using NUPACK23 and attributed a folding score (FSi) measuring the similarity between the predicted RNA secondary structure and that of the target ASO-iSBH [4]. The ensuing pool is then sorted and only the fittest sequences are considered for the next iteration (repeat steps [1] to [4]). Once a user-defined criteria based on score or number of iterations is reached, the system outputs a list of top sensing-loop candidates.

FIG. 13: iSBH-mediated assembly of transcriptional programs on endogenous genes

(a) Schematic diagram detailing the SAM system for single sgRNA CRISPR-mediated transcriptional activation of endogenous target genes [7]. dCas9-VP64 is complexed with a modified sgRNA-2XMS2 (red hairpins) capable of recruiting additional 4×p65-HSF1 effectors through MS2-MCP interactions. (b-e) Implementation of iSBH technology for engineering branching and orthogonal gene network modules on endogenous targets. (b) Concurrent overexpression of two target genes (HBG1 and IL1B) using Csy4-responsive iSBH-sgRNAs (branching module). Co-transfection of iSBH^{(0B)} SAM-Csy4^{(nano)}HBG1, iSBH^{(0B)} SAM-Csy4^{(nano)}IL1B with SAM system components elicits an increase in the corresponding genes transcript levels in the presence of Csy4 compared to nv-SCR or decoy inducer controls. (c) Wiring of HBG1 and MB gene output with independent inducers (Csy4 and Cas6A respectively). Co-expression of iSBH^{(0B)} SAM-Csy4^{(nano)} HBG1 and iSBH^{(0B)}SAMCas6A^{(medium)}IL1B leads to inducer-specific orthogonal regulation of transcriptional gene output compared to nv-SCR control. (d) Conditional overexpression of HBG1 and MB using a single ASO. iSBH^{(0B)}SAM-ASOε-HBG1 and iSBH^{(0B)}SAM-ASOε-IL1B responsive to a shared trigger ASOε were used to implement a branching module. Delivery of ASOε 24 h post-transfection resulted in an increase in transcript levels for both genes compared to nv-SCR control, a decoy ASO, or iSBH sgRNAs containing mutant sensing loops (iSBH^{(0B)}SAM-ASOm-HBG1, iSBH^{(0B)}SAM-ASOm-IL1B). (e) iSBH^{(0B)}SAM-ASOλ-HBG1 and iSBH^{(0B)}SAM-ASOτ-IL1B containing different ASO-sensing loops were supplemented with decoy ASO, ASOλ, ASOτ or a combination of ASOλ and ASOτ. Orthogonal regulation of HBG1 and MB transcription was observed in the presence of matching ASOs compared to a control nv-SCR sgRNA. For all panels, data displays fold change in transcript levels measured by RT-qPCR (n=3 biological replicates (×3 technical replicates), mean+/−SD). Cas9-VP64 and MCP-p65-HSF1 were co-transfected by default in all conditions. For additional controls see also FIG. 14.

FIG. 14: Evaluation of iSBH sgRNA specificity.

(a-d) Additional experimental data supporting the generation of protein- and ASO-responsive branching and orthogonal modules on endogenous targets (see FIG. 13). Csy4-iSBH-sgRNAs (a), Cas6A-iSBH-sgRNAs (b) and ASO-iSBH-sgRNAs (c, d) were tested separately for conditional activation of each corresponding target gene. Data show fold changes in gene transcript levels (compared with scramble sgRNA) measured by RT-qPCR (n=3 biological replicates (×3 technical replicate), mean+/−SD). sgRNAs and inducer transfection schemes are shown under each condition. dCas9-VP64 and MCP-p65-HSF1 constructs were transfected by default in all conditions.

FIG. 15: HHRz-based iSBH spacer release mechanism.

(a) Theoretical framework underlying the use of allosteric hammerhead ribozymes (aHHRz) for iSBH-sgRNA-based CRISPR-TA conditional control of gene expression. An iSBH equipped with an engineered aHHRz containing a ligand-sensing aptamer domain is delivered to cells. In the absence of a cognate ligand (OFF-state) the aHHRz assumes an inactive conformation, sequestering the spacer and inhibiting CRISPR-TA. Delivery of the cognate ligand promotes a change in conformation (strand displacement), which allows the aHHRZ to resume its catalytic activity. aHHRz cleavage facilitates back-fold dissociation from the spacer, rendering the sgRNA competent for CRISPR-TA-mediated activation of its programmed target gene (ON-state). (b) Sequence and RNA secondary structure of a prototype HHRz-SBH. The active HHRz is directly fused to the SBH^{(0B)}CTS1. A to G point mutation (yellow) renders the HHRz inactive (mHHRz). (c) Representative CRISPR-TA assay flow cytometry scatter plots with nv-SCR, SBH^{(0B)} HHRz-CTS1, and SBH^{(0B)} mHHRz-CTS1 sgRNAs (EYFP reporter fluorescence against iBlue sgRNA transfection).

FIG. 16:

Catalogue of silent (SBH) and inducible (iSBH) designs used in the study organised by inducer and target type.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Controls Without SBH

To evaluate the potential of SBH-based systems to control CRISPR transcription activators (CRISPR-TA), we first adopted a recently-developed reporter assay, which displays potent transgene activation using a single sgRNA [13]. This system relies on targeting a dCas9-effector fusion protein (dCas9-VP64) to an ‘enhancer-like’ region containing 8×CRISPR target site (CTS) repeats placed upstream of a fluorescent reporter gene (FIG. 2). As anticipated, independent co-transfection of dCas9-VP64 with either of two native sgRNAs (nv-CTS1 and nv-CTS2) programmed to target distinct CTS enhancers (CTS1 and CTS2 respectively) showed strong spacer-specific reporter activation, which was not observed with the control scramble spacer (FIG. 1b).

### Example 2: SBH Prevents Expression

To assess if the hairpin architecture could silence CRISPR-TA activity, we designed prototype SBHs for both CTS1- and CTS2-targeting sgRNAs whereby the back-fold extension covered the entire spacer segment (0 free spacer nucleotides, SBH⁽⁰⁾CTS-sgRNA) as well as the first scaffold nucleotide (FIG. 1c). Indeed, in contrast to nv-CTS sgRNAs, the corresponding SBH⁽⁰⁾CTS-sgRNAs fully abrogated CRISPR-TA activity resulting in no detectable reporter gene expression, irrespective of the guide spacer sequence used (CTS1 or CTS2) (FIG. 1b). To validate backfold:spacer base pairing as the cause of silencing, we designed three control constructs recapitulating the length and/or structure of the SBH extension without pairing to spacer nucleotides. Confirming that simple 5′ end guide extension is not sufficient to block CRISPR-TA activity, all three controls (offset 10 bp hairpin, SBH^{(ctrl-1)}CTS; offset 20 bp hairpin, SBH^{(ctrl-2)}CTS and scrambled back-fold extension, SBH^{(ctrl-3)}CTS) displayed reporter activation for both CTS1 and CTS2 (FIG. 1b, FIG. 3a, b).

### Example 3: How Much of Spacer is Covered

We found that progressive shortening of the back-fold extension led to a corresponding increase in CRISPR-TA activity as a result of releasing 5 (SBH⁽⁵⁾CTS2), 10(SBH⁽¹⁰⁾CTS2) and 15(SBH⁽¹⁵⁾CTS2) spacer nucleotides from its 3′ end (proximal to the tracrRNA) (FIG. 1d-f). In contrast, the same effect was not observed when shortening the sgRNA spacer to corresponding lengths, which maintained native levels of activation at 15 and 10 nucleotides (FIG. 4a-c) [14]. This data suggests that SBH-sgRNAs with variable spacer coverage could potentially be employed for tuning gene expression output.

### Example 4: Types of Hairpin

The strong repressive properties displayed by the back-fold extension design provide an ideal framework for evolving inducible SBH (iSBH) systems. Based on the modular SBH architecture, the connecting loop sequence can in principle be replaced for sensor-actuator cleaving units to create interchangeable spacer-release mechanisms. To thermodynamically favour back-fold extension removal post-cleavage while maintaining full silencing in the OFF-state, we first designed bulged SBH structures to reduce melting temperatures and promote low-energy stepwise strand separation (FIG. 5a). Relative to SBH⁽⁰⁾CTS1, insertion of two 2 nt bulges in the stem structure (SBH^{(0B)}CTS1, where 0=complete spacer coverage and B=bulge stem) maintained full CRISPR-TA inhibition while increasing the predicted structural free energy from −38.4 to −23.7 kcal/mol (FIG. 5b, c). In contrast, the presence of an additional basal bulge (SBH^{(0B*)}CTS1; G=−15.0 kcal/mol) destabilised the stem leading to loss of SBH-mediated silencing (FIG. 5b, c). Therefore, the SBH^{(0B)}CTS design was used as the default stem architecture for subsequent iSBH implementations.

### Example 5: Csy4

We adapted the SBH platform to couple the transcriptional output of a target gene with protein-based inducers. Csy4 is a CRISPR-associated endoribonuclease from Pseudomonas aeruginosa which recognises and cleaves the 16 nt core of a 28 nt RNA stem-loop [15, 16]. The Csy4 gene has been codon optimised and used in mammalian cells for multiplexed delivery of sgRNAs from a single Pol-II expressed transcript [17]. To demonstrate conditional CRISPR-TA activation, we engineered a Csy4-responsive iSBH by grafting its cognate RNA motif to SBH^{(m)}CTS (iSBH^{(0B)}Csy4^{(full)}CTS) (FIG. 6a, b). In parallel, a control iSBH construct was generated containing a single G-C point mutation in the Csy4 recognition sequence (iSBH^{(0B)}Csy4m^{(full)}CTS), which was previously reported to prevent cleavage of the RNA target (FIG. 6b) [15]. As expected, CRISPR-TA was completely silenced in the OFF-state (decoy empty plasmid), while analysis of ON-state reporter expression revealed robust Csy4-mediated CRISPR-TA activation (FIG. 6c). Confirming the specificity of this effect, the single G-C point mutation in the recognition sequence rendered the iSBH system insensitive to Csy4 induction (FIG. 6c).

### Example 6: Optimised Loops

We performed an iterative optimisation of the iSBH design, which aimed to further lower the stem separation free energy and reduce the number of unstructured 5′ residual nucleotides not bound by dCas9 following Csy4 cleavage. This was accomplished by fusing the Csy4 RNA motif with either the distal or proximal SBH^{(0B)}CTS bulge (FIG. 6d). The resulting designs, iSBH^{(0B)}Csy4^{(medium)}CTS1 and iSBH^{(0B)}Csy4^{(nano)}CTS1 had a predicted decrease in stem stability (−27.7 and −22.7 kcal/mol relative to −31.5 for iSBH^{(0B)}Csy4^{(full)}CTS1) and correspondingly, displayed an increase in reporter expression fold-change (˜9e3 and ˜45e3 relative to ˜3e3) (FIG. 6e, f). This effect was consistent for both CTS1 and CTS2 targeting sgRNAs (FIG. 6f, FIG. 7a-c). The) iSBH^{(0B)}Csy4^{(nano)}CTS showed the most robust ON-state activation without any detectable loss of silencing efficiency in the OFF-state (FIG. 6e, f). This is consistent with the observation that shorter sgRNA spacers tend to promote higher CRISPR-TA activation than full-length (20 nt) spacers (FIG. 4a-c) [14]. These results demonstrate that endoribonucleases are effective iSBH-sgRNA inducers that could be genetically encoded to create pre-programmed prosthetic synthetic circuits in living cells.

### Example 7: Antisense Oligonucleotides

Antisense oligonucleotides (ASOs) have emerged as a highly versatile class of compounds that can be safely and efficiently delivered in both cells and organisms to alter gene expression (up and down-regulation) and to interfere with post-transcriptional RNA processing (splicing, microRNA regulation, etc) [18, 19]. To expand the scope of the iSBH toolkit, we sought to engineer spacer release mechanisms responsive to short ASOs, thus providing a means for temporal exogenous control of CRISPR-TA. In addition, we reasoned that the sequence diversity available for ASO designs would supply an extensive repertoire of possible inducer:target combinations. Conceptually, this strategy relies on the ability of ssDNA ASOs to bind complementary iSBH sensing loops and engage nuclear RNase-H mediated cleavage of the RNA strand in the resulting DNA:RNA hybrid, thus releasing back-fold-mediated CRISPR-TA silencing (FIG. 6g). To establish the feasibility of this approach, ASO inducers were delivered 24 hours following transfection of core system components (dCas9-VP64, sgRNA, reporter), and CRISPR-TA-induced reporter expression was assessed one day later.

Previous studies have shown that ASO-mediated RNase-H cleavage efficiency positively correlates with target site accessibility [20, 21]. Based on these considerations, ASO-responsive iSBHsgRNAs were designed to limit structural interactions within the sensing domain, thus constraining the loop in an open conformation. Similar to Csy4-responsive iSBH, grafting a 14 nt ASO-sensing loop onto the SBH^{(0B)}CTS retained full OFF-state silencing in the presence of a decoy scrambled ASO (FIG. 6h, iSBH^{(0B)} ASOα-CTS2). Demonstrating conditional CRISPR-TA activation, delivery of a 14 nt ASO complementary to the sensing-loop (ASOα-14) rendered a 63-fold increase in reporter expression (FIG. 6i top). Further extension of ASO length and hybridization footprint aimed at favouring strand separation revealed a substantial increase in ON-state CRISPR-TA activity with a 20 nt ASO (ASOα; 473 fold-change), while a 25 nt ASO provided a more moderate gain (ASOα-25; 273 fold-change) (FIG. 6i, j). Control experiments using a scrambled sensing loop (iSBH^{(0B)} ASOm-CTS) rendered the system insensitive to trigger ASOs, recapitulating the effects observed using decoy ASOs and demonstrating the specificity of iSBH:inducer pairs for both CTS1 and CTS2 sgRNAs (FIG. 6k, l). Interestingly, in contrast to Csy4-iSBH designs, fusing the sensing loop to the SBH^{(0B)}CTS distal bulge reduced CRISPR-TA activity, presumably due to dCas9 interfering with ASO:sensing loop hybridization (FIG. 8a-c). In general, we found that Csy4-iSBH designs mediate stronger CRISPR-TA induction than their ASO-responsive counterparts (see FIG. 6). This might be attributable to the fact that, as previously reported, Csy4 remains bound to the 3′ end of the cleaved product [15], and thus could promote more effective strand separation of the back-fold structure.

### Example 8: Gene Networks with Csy4 and Cas6A

Leveraging the versatility and simplicity of the iSBH design, we next sought to test its potential as a framework for the assembly of gene networks (GN). Complex synthetic gene circuits can in principle be reduced to two fundamental GN modules: the branching module and ii) the orthogonal module. In the branching module a single upstream event (inducer) simultaneously controls the activity of multiple downstream nodes. On the other hand, an orthogonal module allows asynchronous control of downstream targets using independent inducer:gene pairs (FIG. 9a). Successful implementation of these modules for both protein- and ASO-responsive iSBH designs can be directly evaluated using a simple dual reporter assay ([8×CTS1-EYFP]-[8×CTS2-ECFP]).

To assemble a branching module using protein-responsive iSBH-sgRNAs and demonstrate simultaneous activation of two target genes conditioned on the presence of Csy4, we co-transfected iSBH^{(0B)}Csy4^{(nano)}CTS1 and iSBH^{(0B)}Csy4^{(nano)}CTS2 along with dCas9-VP64 and the dual reporter system. Both target genes displayed robust CRISPR-TA-mediated expression in the presence of Csy4, while no detectable activation was observed in the absence of inducer (FIG. 9b, FIG. 10a). Furthermore, sequentially scrambling the spacer sequence of each iSBHsgRNA resulted in the expression of a single target gene, validating the dependence of branched activation on the presence of both guides (FIG. 9b, FIG. 10a).

Next, we designed an iSBH-based orthogonal gene module implementing independent protein inducer:target gene pairs. Using the same strategy previously employed for Csy4, we created full, medium and nano iSBH-sgRNAs responsive to the CRISPR endoribonuclease Cas6A from Thermus thermophilus, by grafting its cognate RNA motif onto SBH^{(0B)}CTS (FIG. 11a) [22]. Similar to Csy4-iSBH-sgRNAs, Cas6A-responsive hairpins showed full CRISPR-TA silencing in the OFF-state and robust ON-state target gene activation (FIG. 11b, c). Following optimization studies, the iSBH^{(0B)}Csy4^{(nano)}CTS1 and iSBH^{(0B)} Cas6A^{(medium)}CTS2 designs (optimal ON/OFF-state characteristics) were selected to condition the expression of two target genes (EYFP, ECFP) on the presence of Csy4 and Cas6A respectively. Demonstrating successful implementation of an orthogonal module, each target gene was exclusively activated in the presence of its corresponding trigger (Csy4 or Cas6A), with no detectable crosstalk between independent branches (FIG. 9c, FIG. 10b). As expected, simultaneous expression of both reporter genes was achieved when Csy4 and Cas6A were co-transfected (FIG. 9c, FIG. 10b).

### Example 9: Gene Networks With ASO

We then set out to implement corresponding branching and orthogonal modules exogenously controlled by ASO triggers. ASO-mediated branching requires the evolution of a shared sensing-loop, which should display optimal folding properties across multiple iSBH-sgRNA spacer sequences. To automate this process we have created iSBHfold, a custom software combining genetic algorithm with RNA secondary structure predictions [23], and used it to engineer iSBH^{(0B)} ASOβ-CTS1 and iSBH^{(0B)} ASOβ-CTS2 sgRNAs sharing a unique sensing-loop (FIG. 12). The corresponding module displayed ON-state branching behaviour following delivery of cognate inducer ASOβ, and complete silencing in the presence of a decoy ASO (scramble sequence) (FIG. 9d, FIG. 10c). Furthermore, control experiments aimed to decouple each target gene from the inducer by sequentially mutating the iSBH^{(0B)} ASOβ-CTS1 or iSBH^{(0B)} ASOβ-CTS2 sensing loops, revealed the expected loss of reporter gene activation in the corresponding branch (FIG. 9d, FIG. 10c).

The availability of a broad inducer pool for ASO-responsive iSBH sgRNAs provides an optimal framework for the construction of CRISPR-TA-based orthogonal gene modules in mammalian cells. We assembled a simple orthogonal module which couples conditional activation of two reporter target genes (EYFP and ECFP) with distinct ASO inducers (ASOβ and ASOα). Conditional activation of quiescent iSBH^{(0B)} ASOβ-CTS1 and iSBH^{(0B)} ASOα-CTS2 sgRNAs with separate or simultaneous delivery of ASOβ and ASOα, resulted in the anticipated target gene activation profiles without any apparent interference between individual branches (FIG. 9e, FIG. 10d). Together these results demonstrate the relevance of the iSBH framework in facilitating assembly of basic modules for construction of synthetic gene circuits.

### Example 10: Synergistic Activation Mediators

Next we sought to establish if iSBH solutions could be leveraged to enhance state of the art CRISPR-TA methods, allowing implementation of branching and orthogonal control of endogenous target genes. Recently, several CRISPR-TA systems have been reported which provide alternatives to sgRNA promoter tiling, enabling potent gene activation with a single sgRNA [7, 24-26]. Notably, since iSBH designs are based on minimal engineering of the guide RNA 5′ end, they are virtually inconsequential to sgRNA integrity in the ON-state. Therefore, this framework should be compatible with all previously reported CRISPR derivatives.

The synergistic activation mediator (SAM) system provides an elegant solution to enable robust transcriptional activation of endogenous genes by maximizing the number of effector domains associated with one dCas9-sgRNA complex (FIG. 13a) [7]. We first engineered the SAM sgRNA (containing two MS2 loops) to accommodate Csy4-responsive iSBHs and program parallel conditional activation of the HBG1 and IL1B genes [7]. Delivery of the resulting constructs (iSBH^{(0B)} SAM-Csy4^{(nano)}HBG1 and iSBH^{(0B)} SAM-Csy4^{(nano)}IL1B respectively) to HEK293-T cells, along with the SAM system, showed robust parallel activation of both genes in the presence of Csy4 relative to endogenous levels (FIG. 13b). Control experiments demonstrated the specificity of target gene activation and efficiency of iSBH-mediated silencing in the absence of inducer (FIG. 13b, FIG. 14a). In contrast, replacing the Csy4 for Cas6A RNA motifs to generate iSBH^{(0B)} SAM-Cas6A^{(nano)}IL1B (FIG. 14b), uncoupled MB from HBG1 (iSBH^{(0B)} SAM-Csy4^{(nano)}HBG1) conditional activation, thus demonstrating orthogonal control of endogenous gene expression (FIG. 13c). As anticipated, concurrent delivery of both Cys4 and Cas6A led to simultaneous activation of their corresponding target genes (FIG. 13c).

In addition, parallel and orthogonal transcriptional modules were programmed to respond to exogenous triggers by applying the ASO-iSBH design rationale to HBG1 and MB SAM sgRNAs. First, a shared ASO sensing-loop compatible with both HBG1 and IL1B spacers was evolved using the iSBHfold custom-made algorithm. The resulting iSBH^{(0B)}SAM-ASOε-HBG1 and iSBH^{(0B)}SAM-ASOε-IL1B sgRNAs were tested for conditional activation of the corresponding genes following delivery (24 h post-transfection) of matching ASO. Analysis of transcript levels revealed a significant parallel increase in the expression of both genes (FIG. 13d, FIG. 14c). In contrast, negligible alterations in gene output were observed when providing a decoy ASO or by decoupling trigger ASO and iSBHs following mutagenesis of the sensing segments. Finally, we designed two new ASO sensing-loops and combined them with the HBG1 and IL1B iSBH^{(0B)} SAM scaffolds, to generate iSBH^{(0B)}SAM-ASOλ-HBG1 and iSBH^{(0B)}SAM-ASOτ-IL1B respectively. Using these constructs we demonstrate endogenous ASO-dependent orthogonal module behaviour in which the transcriptional output of each gene is controlled independently of the other (FIG. 13e, FIG. 14d).

### Example 11: Other Ligands

Although endoribonucleases and ASOs offer a rich repertoire of genetically encoded and externally delivered inducers, iSBH-mediated conditional sgRNA activation is not limited to these systems. Theoretically, iSBH-sensing modules could be evolved to respond to other categories of ligands using self-contained cleavage units in the form of allosteric hammerhead ribozymes (aHHRz) (FIG. 15a). Previous studies have shown that aHHRz can be effectively used for the construction of ligand-controlled synthetic circuits [27-29]. To establish the feasibility of leveraging the HHRz design as a self-contained spacer release mechanism, we fused a HHRz structure onto the SBH^{(0B)}CTS scaffold (FIG. 15b). Comparative analysis of SBH-sgRNAs containing catalytically active (SBH^{(0B)} HHRz-CTS1) or inactive (SBH^{(0B)} mHHRz-CTS1) HHRz, demonstrated robust HHRz-mediated activation of reporter gene expression and complete silencing in the OFF-state (FIG. 15c). Future iterations of this generic HHRz-SBH scaffold could take advantage of established in vivo/in vitro RNA aptamer evolution strategies to engineer iSBHs responsive to a variety of protein, nucleotide and small molecule ligands [30-33].

### Example 12: Use of miRNA Response Elements the Cleavable Loop

In this Example, the cleavable loop element comprises a miRNA response element (MRE) which is utilised in an in vitro system to detect cognate miRNAs.

Design of miRNA-Responsive iSBH-sgRNAs

An iSBH which is capable of sensing a chosen miRNA is created by replacing the loop element of the SBH^{(0B)} design with a longer single-stranded RNA segment (sensing loop) whose sequence is fully complementary to the miRNA trigger.

In Vitro System

The in-vitro system comprises two cell lysates:
1) Sensor lysate (SL)—this contains all the machinery required for CRISPR-based transcription regulation (i.e. CRISPR-TR, dCas9-VP64, reporter genes) as well as the miRNA-responsive iSBH-sgRNAs; and
2) Patient lysate (PL)—this is obtained from the patient's blood and contains or not, depending on the disease state, the miRNAs of interest (disease signature) loaded into a catalytically-active miRISC complex.

The SLs, which are designed to survey particular miRNA profiles, are prepared in advance and snap-frozen for later usage. Upon collection, the patient blood sample is processed into a PL using a standard protocol. The corresponding SLs are activated at 37° C., i.e. a temperature at which all of the system components are able to respond to miRNA-mediated slicing. The PL is mixed with the corresponding SLs and incubated at 37° C. for a period of approximately 1 to 4 hours. The presence in the PL of a miRNA matching the sensing loop sequence of one iSBH-sgRNAs, results in Ago-mediated back-fold removal. The activated sgRNA is then able to drive—in combination with dCas9-VP64—the expression of a fluorescent reporter gene (e.g. EGFP). Reporter fluorescence is then monitored in an array format with a plate reader. Alternatively, reporter proteins that change colour in the human visual spectrum are used, thus alleviating the need for plate readers (i.e. direct detection on the diagnostic paper).

Multiplexing for Complex miRNA Profile Monitoring:

Complex miRNA profiles (involving several miRNAs) are surveyed by using multiple distinct iSBH-sgRNA:reporter pairs. The method is multiplexed by creating a plate (or filter paper with freeze dried spots) where each well is loaded with a different SL designed to report on one specific miRNA. The PL is then added to each well to collect information regarding each miRNA in this panel.

Alternatively, iSBH-sgRNA-reporter pairs are utilised in the same SL if the reporter genes encode fluorescent proteins with distinct excitation/emission spectra; these are deconvoluted using a plate reader with multiple fluorescence detection wavelength.

### REFERENCES

1. Lienert, F., Lohmueller, J. J., Garg, A. & Silver, P. A. Synthetic biology in mammalian cells: next generation research tools and therapeutics. Nature reviews. Molecular cell biology 15, 95-107 (2014).
2. Chavez, A. et al. Comparison of Cas9 activators in multiple species. Nature methods 13, 563-567 (2016).
3. Nunez, J. K., Harrington, L. B. & Doudna, J. A. Chemical and Biophysical Modulation of Cas9 for Tunable Genome Engineering. ACS chemical biology 11, 681-688 (2016).
4. Esvelt, K. M. et al. Orthogonal Cas9 proteins for RNA-guided gene regulation and editing. Nature methods 10, 1116-1121 (2013).
5. Jinek, M. et al. A programmable dual-RNA-guided DNA endonuclease in adaptive bacterial immunity. Science 337, 816-821 (2012).
6. Briner, A. E. et al. Guide RNA functional modules direct Cas9 activity and orthogonality. Molecular cell 56, 333-339 (2014).
7. Nissim, L., Perli, S. D., Fridkin, A., Perez-Pinera, P. & Lu, T. K. Multiplexed and programmable regulation of gene networks with an integrated RNA and CRISPR/Cas toolkit in human cells. Molecular cell 54, 698-710 (2014).
8. Haurwitz, R. E., Jinek, M., Wiedenheft, B., Zhou, K. & Doudna, J. A. Sequence- and structure-specific RNA processing by a CRISPR endonuclease. Science 329, 1355-1358 (2010).
9. Dahlman, J. E. et al. Orthogonal gene knockout and activation with a catalytically active Cas9 nuclease. Nature biotechnology 33, 1159-1161 (2015).
10. Walder, R. Y. & Walder, J. A. Role of RNase H in hybrid-arrested translation by antisense oligonucleotides. Proceedings of the National Academy of Sciences of the United States of America 85, 5011-5015 (1988).
11. Bennett, C. F. & Swayze, E. E. RNA targeting therapeutics: molecular mechanisms of antisense oligonucleotides as a therapeutic platform. Annual review of pharmacology and toxicology 50, 259-293 (2010).
12. Konermann, S. et al. Genome-scale transcriptional activation by an engineered CRISPR-Cas9 complex. Nature 517, 583-588 (2015).
13. Niewoehner, O., Jinek, M. & Doudna, J. A. Evolution of CRISPR RNA recognition and processing by Cas6 endonucleases. Nucleic acids research 42, 1341-1353 (2014).
14. Zadeh, J. N. et al. NUPACK: Analysis and design of nucleic acid systems. Journal of computational chemistry 32, 170-173 (2011).
15. Win, M. N. & Smolke, C. D. Higher-order cellular information processing with synthetic RNA devices. Science 322, 456-460 (2008).
16. Aquino-Jarquin, G. & Toscano-Garibay, J. D. RNA aptamer evolution: two decades of SELEction. International journal of molecular sciences 12, 9155-9171 (2011).
17. Liu, Y. et al. Directing cellular information flow via CRISPR signal conductors. Nature methods (2016).
18. Hsu, P. D., Lander, E. S. & Zhang, F. Development and applications of CRISPR-Cas9 for genome engineering. Cell 157, 1262-1278 (2014).
19. Dominguez, A. A., Lim, W. A. & Qi, L. S. Beyond editing: repurposing CRISPR-Cas9 for precision genome regulation and interrogation. Nature reviews. Molecular cell biology 17, 5-15 (2016).
20. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
21. Chen, B. et al. Dynamic imaging of genomic loci in living human cells by an optimized CRISPR/Cas system. Cell 155, 1479-1491 (2013).
22. Mali, P. et al. CAS9 transcriptional activators for target specificity screening and paired nickases for cooperative genome engineering. Nature biotechnology 31, 833-838 (2013).
23. Zalatan, J. G. et al. Engineering complex synthetic transcriptional programs with CRISPR RNA scaffolds. Cell 160, 339-350 (2015).
24. McKenna, A. et al. Whole organism lineage tracing by combinatorial and cumulative genome editing. Science (2016).
25. Lim, W. A. Designing customized cell signalling circuits. Nature reviews. Molecular cell biology 11, 393-403 (2010).
26. Ran, F. A. et al. Genome engineering using the CRISPR-Cas9 system. Nature protocols 8, 2281-2308 (2013).
27. Klauser, B., Atanasov, J., Siewert, L. K. & Hartig, J. S. Ribozyme-based aminoglycoside switches of gene expression engineered by genetic selection in S. cerevisiae. ACS synthetic biology 4, 516-525 (2015).
28. Aricescu, A. R., Lu, W. & Jones, E. Y. A time- and cost-efficient system for high-level protein production in mammalian cells. Acta crystallographica. Section D, Biological crystallography 62, 1243-1250 (2006).
29. Xie, Z., Wroblewska, L., Prochazka, L., Weiss, R. & Benenson, Y. Multi-input RNAi-based logic circuit for identification of specific cancer cells. Science 333, 1307-1311 (2011).

1. An inducible CRISPR RNA comprising:
(i) a spacer-blocking element;
(ii) a cleavable loop element; and
(iii) a CRISPR sgRNA comprising a spacer element;
wherein (i)-(iii) are arranged 5′-3′ in the above order in the inducible CRISPR RNA, wherein the spacer-blocking element has a nucleotide sequence which is at least partially complementary to that of the spacer element, and wherein the spacer-blocking element, cleavable loop element and spacer element are capable of forming a stem-loop structure.

2. An inducible CRISPR RNA as claimed in claim 1, wherein the cleavable loop element consists of a loop structure, a loop-stem-loop structure or a loop-stem-loop-stem-loop structure.

3. An inducible CRISPR RNA as claimed in claim 1 or claim 2, wherein the cleavable loop element is capable of being bound by an oligo-ribonucleotide-binding moiety in a sequence-specific manner.

4. An inducible CRISPR RNA as claimed in claim 3, wherein the cleavable loop element comprises a cleavage site for an endo-ribonuclease.

5. An inducible CRISPR RNA as claimed in claim 4, wherein the endo-ribonuclease is Cas6A, Csy4 or Cpf1.

6. An inducible CRISPR RNA as claimed in claim 1 or claim 2, wherein the cleavable loop element is one which is cleavable by a hammerhead ribozyme, preferably by an allosteric self-cleaving hammerhead ribozyme (aHHRz).

7. An inducible CRISPR RNA as claimed in claim 1 or claim 2, wherein the cleavable loop element is one which is cleavable by nuclear RNAse H after the binding of an antisense ssDNA oligonucleotide to the loop element.

8. An inducible CRISPR RNA as claimed in any one of claims 1-3, wherein the cleavable loop element comprises a miRNA responsive element (MRE).

9. An inducible CRISPR RNA as claimed in any one of the preceding claims, wherein the sequence of the spacer element is complementary to that of a regulatory element, preferably an enhancer, promoter or terminator sequence.

10. An inducible CRISPR RNA as claimed in any one of the preceding claims, wherein one or more functional domains are attached, directly or indirectly, to the inducible CRISPR RNA, preferably to the CRISPR sgRNA.

11. An inducible CRISPR RNA as claimed in claim 10, wherein one or more functional domains are attached via stem-loop RNA binding proteins (RBPs) to the CRISPR sgRNA.

12. An inducible CRISPR RNA as claimed in claim 10 or claim 11, wherein the functional domain is a bacteriophage MS2 coat protein or the Pseudomonas PP7 RNA-binding coat protein.

13. A composition comprising:
(a) an inducible CRISPR RNA as claimed in any one of claims 1 to 12; and
(b) a CRISPR enzyme.

14. A kit comprising:
(a) an inducible CRISPR RNA as claimed in any one of claims 1 to 12; and
(b) a CRISPR enzyme,
in a form suitable for sequential, separate or simultaneous use.

15. A composition as claimed in claim 13 or a kit as claimed in claim 14, wherein the CRISPR enzyme is a catalytically-inactive enzyme, preferably an endonuclease-deficient enzyme, more preferably dCas9 or dCpf1, or a variant or derivative thereof which lacks endonuclease activity.

16. A composition as claimed in claim 13 or a kit as claimed in claim 14, wherein the CRISPR enzyme is a catalytically-active enzyme, preferably an endonuclease-active enzyme, more preferably Cas9 or Cpf1, or a variant or derivative thereof which has endonuclease activity.

17. A DNA molecule encoding an inducible CRISPR RNA as claimed in any one of claims 1 to 12.

18. A vector comprising a DNA molecule as claimed in claim 17.

19. A composition comprising:
(a) a DNA molecule as claimed in claim 17 or vector as claimed in claim 18; and
(b) a DNA molecule or vector encoding a CRISPR enzyme.

20. A kit comprising:
(a) a DNA molecule as claimed in claim 17 or vector as claimed in claim 18; and
(b) a DNA molecule or vector encoding a CRISPR enzyme,
in a form suitable for sequential, separate or simultaneous use.

21. A composition or kit as claimed in any one of claim 13-16 or 19-20, wherein the CRISPR enzyme comprises one or more functional domains which are attached, directly or indirectly, to the CRISPR enzyme.

22. A composition or kit as claimed in any one of claim 13-16 or 19-20, wherein the CRISPR enzyme and the one or more functional domains form a fusion polypeptide.

23. A composition or kit as claimed in any one of claim 13-16 or 19-22, wherein the one or more functional domains have one or more activities selected from the group consisting of methylase activity, demethylase activity, transcription activation activity, transcription repression activity, transcription release factor activity, histone modification activity, RNA cleavage activity, DNA cleavage activity, nucleic acid binding activity and base-conversion activity.

24. A composition or kit as claimed in any one of claim 13-16 or 19-23, wherein the one or more functional domains is a transcription activator.

25. Use of an inducible CRISPR RNA as claimed in any of claims 1-12, a composition as claimed in any one of claim 13, 15-16, 19 or 21-24, a kit as claimed in claim 14 or 20, a DNA as claimed in claim 17 or a vector as claimed in claim 18, for genome editing, epigenetic alteration, base editing, DNA labelling, base-conversion or lineage tracing throughout development or in disease states.

26. A method for inducibly targeting a CRISPR complex to a target DNA in a host cell, the method comprising the steps:
(i) expressing in the host cell:
(a) a first inducible CRISPR RNA as claimed in any one of claims 1-12, wherein the nucleotide sequence of the spacer element is fully or partially complementary to a region of the first target DNA; and
(b) a CRISPR enzyme,
such that the first inducible CRISPR RNA and CRISPR enzyme form a CRISPR complex; and
(ii) inducing, at a desired time, cleavage of the cleavable loop element of the first inducible CRISPR RNA,
thus allowing the spacer element to target the CRISPR complex to the first target DNA.

27. A method as claimed in claim 26, wherein the method additionally comprises the steps:
(i) expressing in the host cell:
(a) a plurality of inducible CRISPR RNAs as claimed in any one of claims 1-12, wherein the nucleotide sequences of the spacer elements are independently fully or partially complementary to regions of the plurality of target DNAs; and
(b) a CRISPR enzyme,
such that the plurality of inducible CRISPR RNAs and CRISPR enzymes form a plurality of CRISPR complexes; and
(ii) inducing, at a desired time, cleavage of the cleavable loop elements of the plurality of inducible CRISPR RNAs,
thus allowing the spacer elements to target the plurality of CRISPR complexes to the plurality of target DNAs.

28. A method for inducibly targeting a functional domain to a target DNA in a host cell, the method comprising the steps:
(i) expressing in the host cell:
(a) a first inducible CRISPR RNA as claimed in any one of claims 1-12, wherein the nucleotide sequence of the spacer element is fully or partially complementary to a region of the first target DNA; and
(b) a CRISPR enzyme,
such that the first inducible CRISPR RNA and CRISPR enzyme form a first CRISPR complex, wherein the first CRISPR complex comprises one or more functional domains; and
(ii) inducing, at a desired time, cleavage of the cleavable loop element of the first inducible CRISPR RNA,
thus allowing the spacer element to direct binding of the first CRISPR complex to the target DNA and thus targeting the one or more functional domains to the first target DNA.

29. A method as claimed in claim 28, wherein the method additionally comprises the steps:
(i) expressing in the host cell:
(a) a second inducible CRISPR RNA as claimed in any one of claims 1-12, wherein the nucleotide sequence of the spacer element is fully or partially complementary to a region of a second target DNA; and
(b) a CRISPR enzyme,
such that the second inducible CRISPR RNA and CRISPR enzyme form a second CRISPR complex, wherein the second CRISPR complex comprises one or more functional domains; and
(ii) inducing, at a desired time, cleavage of the cleavable loop element of the second inducible CRISPR RNA,
thus allowing the spacer element to direct binding of the second CRISPR complex to the second target DNA, thus targeting the one or more functional domains to the second target DNA.

30. A method as claimed in any one of claims 26-29, wherein the CRISPR enzyme is a catalytically-active enzyme, preferably an endonuclease-active enzyme, more preferably Cas9 or Cpf1, or a variant or derivative thereof which has endonuclease activity.

31. A method as claimed in any one of claims 26-29, wherein the CRISPR enzyme is a catalytically-inactive enzyme, preferably an endonuclease-deficient enzyme, more preferably dCas9 or dCpf1, or a variant or derivative thereof which lacks endonuclease activity.

32. A method as claimed in any one of claims 28-31, wherein at least one of the one or more functional domains have one or more activities selected from the group consisting of methylase activity, demethylase activity, transcription activation activity, transcription repression activity, transcription release factor activity, histone modification activity, RNA cleavage activity, DNA cleavage activity, nucleic acid binding activity and base-conversion activity.

33. A method as claimed in any one of claims 26-32, wherein cleavage of the cleavable loop elements of the first and second inducible CRISPR RNAs is inducible by the same inducer, preferably wherein the first and second inducible CRISPR RNAs (independently) comprise the same cleavable loop element.

34. A method as claimed in any one of claims 26-32, wherein cleavage of the cleavable loop elements of the first and second inducible CRISPR RNAs is inducible by different inducers (preferably wherein the first and second inducible CRISPR RNAs comprise different cleavable loop elements).

35. A method for inducible editing of a target gene in a target DNA in a host cell, the method comprising the steps:
(i) expressing in the host cell:
(a) an inducible CRISPR RNA as claimed in any one of claims 1-12, wherein the nucleotide sequence of the spacer element is fully or partially complementary to a region of the target gene; and
(b) a CRISPR enzyme with catalytic activity,
such that the inducible CRISPR RNA and CRISPR enzyme form a CRISPR complex; and
(ii) inducing, at a desired time, cleavage of the cleavable loop element of the inducible CRISPR RNA,
thus allowing the spacer element to direct binding of the CRISPR complex to the target gene and thereby inducing editing of the target gene.

36. A method for inducing epigenetic modification of a target DNA in a host cell, the method comprising the steps:
(i) expressing in the host cell:
(a) an inducible CRISPR RNA as claimed in any one of claims 1-12, wherein the nucleotide sequence of the spacer element is fully or partially complementary to a region of the target DNA; and
(b) a catalytically-inactive CRISPR enzyme,
such that the inducible CRISPR RNA and CRISPR enzyme form a CRISPR complex, wherein the CRISPR complex comprises one or more domains which are capable of epigenetic modification of the target DNA; and
(ii) inducing, at a desired time, cleavage of the cleavable loop element of the inducible CRISPR RNA,
thus allowing the spacer element to direct binding of the CRISPR complex to the target DNA;
thus targeting the one or more domains which are capable of epigenetic modification of the target DNA to the region of the target DNA and thereby inducing epigenetic modification of the target DNA.

37. A method for inducible editing of one or more nucleotides of a target DNA in a host cell, the method comprising the steps:
(i) expressing in the host cell:
(a) an inducible CRISPR RNA as claimed in any one of claims 1-12, wherein the nucleotide sequence of the spacer element is fully or partially complementary to a region of the target DNA; and
(b) a catalytically-inactive CRISPR enzyme,
such that the inducible CRISPR RNA and CRISPR enzyme form a CRISPR complex, wherein the CRISPR complex comprises one or more effector domains which have nucleotide-editing properties; and
(ii) inducing, at a desired time, cleavage of the cleavable loop element of the inducible CRISPR RNA,
thus allowing the spacer element to direct binding of the CRISPR complex to the target DNA and thereby targeting the one or more effector domains which have nucleotide-editing properties to the region of the target DNA, thus editing one of more nucleotides of the target DNA.

38. A method for inducible labelling of a target DNA in a host cell, the method comprising the steps:
(i) expressing in the host cell:
(a) an inducible CRISPR RNA as claimed in any one of claims 1-12, wherein the nucleotide sequence of the spacer element is fully or partially complementary to a region of the target DNA; and
(b) a catalytically-inactive CRISPR enzyme,
such that the inducible CRISPR RNA and CRISPR enzyme form a CRISPR complex, wherein the CRISPR complex comprises one or more labelled domains; and
(ii) inducing, at a desired time, cleavage of the cleavable loop element of the inducible CRISPR RNA,
thus allowing the spacer element to direct binding of the CRISPR complex to the target DNA and thereby targeting the one or more labelled domains to the region of the target DNA, thus labelling the target DNA.

39. A method for lineage tracing of daughter cells derived from a host cell, wherein the host cell comprises a first genetic barcode comprising a plurality of repeats of a first target DNA, the method comprising the steps:
(i) expressing in the host cell:
(a) a first inducible CRISPR RNA as claimed in any one of claims 1-12, wherein the nucleotide sequence of the spacer element is fully or partially complementary to a region of a first target DNA in the host cell; and
(b) a catalytically-active CRISPR enzyme,
such that the first inducible CRISPR RNA and CRISPR enzyme form a CRISPR complex; and
(ii) inducing, at a desired time, cleavage of the cleavable loop element of the first inducible CRISPR RNA,
thus allowing the spacer element to direct binding of the CRISPR complex to the first target DNA and wherein the CRISPR enzyme produces one or more mutations in the first genetic barcode which are transmitted to daughter cells, and which mutations can be used to characterise the lineage of the daughter cells.

40. A method as claimed in claim 39, wherein the method additionally comprises the steps of:
(i) expressing in the host cell:
(a) a second inducible CRISPR RNA as claimed in any one of claims 1-12, wherein the nucleotide sequence of the spacer element is fully or partially complementary to a region of a second target DNA in the host cell, wherein the second target DNA forms part of a second genetic barcode comprising a plurality of repeats of the second target DNA; and
(b) a catalytically-active CRISPR enzyme,
such that the second inducible CRISPR RNA and CRISPR enzyme form a second CRISPR complex; and
(ii) inducing, at a desired time, cleavage of the cleavable loop element of the second inducible CRISPR RNA,
thus allowing the spacer element to direct binding of the second CRISPR complex to the second target DNA and wherein the CRISPR enzyme produces one or more mutations in the second genetic barcode which are transmitted to daughter cells, and which mutations can be used to characterise the lineage of the daughter cells.

41. A method for lineage tracing of daughter cells derived from a host cell, the method comprising the steps:
(i) expressing in the host cell:
(a) a first inducible CRISPR RNA as claimed in any one of claims 1-12 from the genome of the host cell, wherein the CRISPR sgRNA is associated with a PAM sequence; and
(b) a catalytically-active CRISPR enzyme,
such that the first inducible CRISPR RNA and CRISPR enzyme form a first CRISPR complex; and
(ii) inducing, at a desired time, cleavage of the cleavable loop element of the first inducible CRISPR RNA,
thus allowing the spacer element to direct binding of the first CRISPR complex to the genomic DNA which encodes the first CRISPR sgRNA and wherein the CRISPR enzyme produces one or more mutations in the genomic DNA which encodes the first CRISPR sgRNA which mutations are transmitted to daughter cells, and which mutations can be used to characterise the lineage of the daughter cells.

42. A method as claimed in claim 41, wherein the method additionally comprises the steps of:
(i) expressing in the host cell:
(a) a second inducible CRISPR RNA as claimed in any one of claims 1-12 from the genome of the host cell, wherein the CRISPR sgRNA is associated with a PAM sequence; and
(b) a catalytically-active CRISPR enzyme,
such that the second inducible CRISPR RNA and CRISPR enzyme form a second CRISPR complex; and
(ii) inducing, at a desired time, cleavage of the cleavable loop element of the second inducible CRISPR RNA,
thus allowing the spacer element to direct binding of the second CRISPR complex to the genomic DNA which encodes the second CRISPR sgRNA and wherein the CRISPR enzyme produces one or more mutations in the genomic DNA which encodes the second CRISPR sgRNA which mutations are transmitted to daughter cells, and which mutations can be used to characterise the lineage of the daughter cells.

43. A method for inducing transcription of a target gene in a target DNA in a host cell, the method comprising the steps:
(i) expressing in the host cell:
(a) an inducible CRISPR RNA as claimed in any one of claims 1-12, wherein the nucleotide sequence of the spacer element is fully or partially complementary to a region of the target DNA in the vicinity of the target gene; and
(b) a catalytically-inactive CRISPR enzyme,
such that the inducible CRISPR RNA and CRISPR enzyme form a CRISPR complex, wherein the CRISPR complex comprises one or more effector domains; and
(ii) inducing, at a desired time, cleavage of the cleavable loop element of the inducible CRISPR RNA,
thus allowing the spacer element to direct binding of the CRISPR complex to the target DNA and thereby targeting the one or more effector domains to the region of the target DNA in the vicinity of the target gene, thus inducing transcription of the target gene.

44. A method for inducing coordinated transcription of two or more target genes in one or more target DNAs in a host cell, the method comprising the steps:
(i) expressing in the host cell:
(a) an inducible CRISPR RNA as claimed in any one of claims 1-12, wherein the nucleotide sequence of the spacer element is independently fully or partially complementary to a region of one or more target DNAs in the vicinity of the two or more target genes; and
(b) a catalytically-inactive CRISPR enzyme,
such that the inducible CRISPR RNA and CRISPR enzyme form a CRISPR complex, wherein the CRISPR complex comprises one or more effector domains; and
(ii) inducing, at a desired time, cleavage of the cleavable loop element of the inducible CRISPR RNA,
thus allowing the spacer element to direct binding of the CRISPR complex to the two or more target DNAs and thereby targeting the one or more effector domains to the regions of the target DNAs in the vicinity of the target genes, and thus inducing coordinated transcription of the two or more target genes.

45. A method for inducing coordinated transcription of two or more target genes in one or more target DNAs in a host cell, the method comprising the steps:
(i) expressing in the host cell:
(a) two or more different inducible CRISPR RNAs as claimed in any one of claims 1-12, wherein the nucleotide sequences of the spacer elements are independently fully or partially complementary to regions of one or more target DNAs in the vicinity of the two or more different target genes, and wherein the cleavable loop elements of the two or more different inducible CRISPR RNAs are cleavable by the same inducer; and
(b) a catalytically-inactive CRISPR enzyme,
such that the different inducible CRISPR RNAs and the CRISPR enzyme form different CRISPR complexes, wherein the CRISPR complexes comprise one or more effector domains; and
(ii) inducing, at desired time, cleavage of the cleavable loop elements of the inducible CRISPR RNAs,
thus allowing the spacer elements to direct binding of the CRISPR complexes to the two or more target DNAs at the desired time and thereby targeting the one or more effector domains to the regions of the target DNAs in the vicinity of the target genes, and thus inducing coordinated transcription of the two or more target genes.

46. A method for inducing orthogonal transcription of two or more target genes in one or more target DNAs in a host cell, the method comprising the steps:
(i) expressing in the host cell:
(a) two or more different inducible CRISPR RNAs as claimed in any one of claims 1-12, wherein the nucleotide sequences of the spacer elements are independently fully or partially complementary to regions of one or more target DNAs in the vicinity of the two or more different target genes; and
(b) a catalytically-inactive CRISPR enzyme,
such that the different inducible CRISPR RNAs and the CRISPR enzyme form different CRISPR complexes, wherein the CRISPR complexes comprise one or more effector domains; and
(ii) inducing, at desired times, cleavage of the cleavable loop elements of the inducible CRISPR RNAs,
thus allowing the spacer elements to direct binding of the CRISPR complexes to the two or more target DNAs at the desired times and thereby targeting the one or more effector domains to the regions of the target DNAs in the vicinity of the target genes, and thus inducing orthogonal transcription of the two or more target genes.

47. A method for detecting the presence of a miRNA in a test sample, the method comprising the steps:
(i) contacting a CRISPR complex with the test sample and a reporter DNA,
wherein the CRISPR complex comprises
(a) an inducible CRISPR RNA as claimed in any one of claims 1-12, wherein the nucleotide sequence of the spacer element is fully or partially complementary to a region of the reporter DNA;
and wherein the cleavable loop element comprises a miRNA response element (MRE) which is capable of being bound by the miRNA; and
(b) a CRISPR enzyme,
under conditions such that if the miRNA is present in the test sample, the miRNA will bind to the MRE in the cleavable loop element thus inducing cleavage of the cleavable loop element, thus allowing the spacer element to bind to the region of the reporter DNA, and
(ii) detecting the presence or absence of the reporter gene or reporter gene product, this being indicative of the presence or absence of the miRNA in the test sample.

48. A method for detecting the presence of one or more miRNAs in a test sample, the method comprising the steps:
(i) contacting a plurality of CRISPR complexes with the test sample and one or more reporter DNAs,
wherein the CRISPR complexes each independently comprise
(a) an inducible CRISPR RNA as claimed in any one of claims 1-12, wherein the nucleotide sequence of the spacer element is fully or partially complementary to a region of the one or more reporter DNAs;
and wherein the cleavable loop element comprises a miRNA response element (MRE) which is capable of being bound by one of the miRNAs; and
(b) a CRISPR enzyme,
under conditions such that if one or more of the miRNAs are present in the test sample, those miRNAs will independently bind to a cognate MRE in a cleavable loop element thus inducing cleavage of that cleavable loop element, thus allowing the spacer element to bind to the region of the one or more reporter DNAs, and
(ii) detecting the presence or absence of the one or more reporter genes or one or more reporter gene products, this being indicative of the presence or absence of one or more of the miRNAs in the test sample.

49. An in vivo method of inducing transcription of a target gene in a subject, the method comprising the steps:
(i) expressing an inducible CRISPR RNA as claimed in any one of claims 1-12 and a catalytically-inactive CRISPR enzyme in a host cell, such that the inducible CRISPR RNA and CRISPR enzyme form a CRISPR complex, wherein the CRISPR complex comprises one or more effector domains, and wherein the spacer-blocking element is bound to the spacer element;
(ii) introducing the host cell into a subject;
(iii) introducing into the subject an agent which cleaves the cleavable loop element,
thus allowing the spacer element to direct binding of the CRISPR complex to a target DNA in the subject which is in the vicinity of the target gene and thereby targeting the one or more effector domains to the region of the target DNA in the vicinity of the target gene, and inducing transcription of the target gene in the subject.

50. A method as claimed in any one of claims 26-49, wherein cleavage of one or more of the cleavable loop elements is induced by one or more of the following inducers:
(a) an endo-ribonuclease (preferably Cas6A, Csy4 or Cpf1);
(d) a conformational change in a hammerhead ribozyme;
(c) an antisense oligonucleotide which binds to the cleavable loop element followed by cleavage of the loop element by nuclear RNAse H; or
(d) a miRNA which binds to a MRE in the cleavable loop element, followed by cleavage of the loop element by a miRISC complex.

51. A method as claimed in claim 50, wherein the inducer is under the control of a tissue-specific promoter (preferably a brain-specific promoter).
